(19) United States
(12) Patent Application Publication
(10) Pub. No.: US 2016/0002325 A1
(43) Pub. Date: Jan. 7, 2016

(54) ANTI-CXCL13 ANTIBODIES AND ASSOCIATED EPITOPE SEQUENCES

(71) Applicant: VACCINEX, INC., Rochester, NY (US)

(72) Inventors: Ekaterina Klimatcheva, Webster, NY (US); Mark Paris, Mendon, NY (US); Ernest S. Smith, Ontario, NY (US)

(73) Assignee: Vaccinex, Inc., Rochester, NY (US)

(21) Appl. No.: 14/769,643

(22) PCT Filed: Mar. 8, 2013
(86) PCT No.: PCT/US2013/029870

§ 371 Date: Aug. 21, 2015

(51) Int. Cl. C07K 16/24; C07K 14/52

(52) U.S. Cl. CPC C07K 16/24; C07K 14/521; C07K 2317/34; C07K 2317/31; C07K 2317/92; C07K 2317/76; C07K 2317/24; C07K 2319/00; A61K 2039/505

(57) ABSTRACT

The invention relates to epitopes of CXCL13 and to binding agents, such as antibodies, that specifically bind to CXCL13.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 430857SEQLIST.TXT, created on Mar. 8, 2013, and having a size of 42.5 kilobytes and is filed concurrently with the specification. The sequence listing contained in this ASCII formatted document is part of the specification and is herein incorporated by reference in its entirety.

### FIELD OF THE INVENTION

The invention relates to epitopes of CXCL13 and to binding agents, such as antibodies and antigen binding fragments thereof, that specifically bind to CXCL13.

### BACKGROUND OF THE INVENTION

Homeostatic B Cell-Attracting chemokine 1 (BCA-1), otherwise known as CXCL13 (or ANGIE, BLC, BLR1L, ANGIE2, or Scyb13), is constitutively expressed in secondary lymphoid organs (e.g., spleen, lymph nodes, and Peyer's patches) by follicular dendritic cells (FDCs) and macrophages. See Gunn et al., Nature 391:799-803 (1998) and Carlsen et al., Blood 104(10):3021-3027 (2004). CXCL13 primarily acts through G-protein-coupled CXCR5 receptor (Burkitt's lymphoma receptor 1). CXCR5 is expressed, e.g., on mature B lymphocytes, CD4+ follicular helper T cells (Thf cells), a minor subset of CD8+T cells, and activated tonsillar Treg cells. See Legler et al., J. Exp. Med. 187:655-660 (1998); Forster et al., Blood 84:830-840 (1994); Fazilleau et al., Immunity 30:324-335 (2009); Ansel et al., J. Exp. Med. 190:1123-1134 (1999); Lim et al., J. Clin. Invest. 114(11):1640-1649 (2004); and R. Forster, Chapter in Academic Press Cytokine Reference, August 2000, each of which is herein incorporated by reference in its entirety. CXCL13 can also signal through another chemokine receptor, CXCR3, which is expressed, e.g., by activated T cells, natural killer (NK) cells, dendritic cells, and endothelial cells of medium to large vessels (Jenh et al., Cytokine 15:113-121 (2001); García-López et al., Laboratory Investigation 81:409-418 (2001), each of which is herein incorporated by reference in its entirety).

Generation of B-cells having the potential for autoantibody (antibody against self-antigen) production is common under normal physiological conditions. However, such natural autoantibodies are low affinity IgM antibodies that exhibit wide-spectrum reactivity and a strong preference for soluble self antigens over cell surface antigens (see, e.g., Dichiero et al., J. Immunol. 134(2):765-771 (1985); Cote et al., Proc. Natl. Acad. Sci 83:2959-2963 (1986)). Autoreactive low-affininty B-cells undergo apoptosis and, therefore, are unlikely to present a danger to a healthy organism.

In the absence of infection and during a normal immune response, CXCL13 is involved in the homing of B-cells and follicular B-helper T cells into primary follicles in lymph nodes and spleen; germinal center formation; and lymphoid organogenesis. See, e.g., Forster et al., Cell 87:1037-1047 (1996).

CXCL13 and CXCR5-deficient mice demonstrated impaired development of Peyer patches and lymph nodes due to the lack of organized follicles. See Ansel et al., Nature 406:309-314 (2000). Furthermore, immunization with T-cell-dependent antigen in the context of the CXCL13 knockout phenotype led to the formation of misplaced and abnormally small germinal centres in the lymph nodes and spleens (Ansel et al.).

In a chronically-inflamed environment, ectopic germinal centers form within affected (often non-lymphoid) tissues. CXCL13 over-expression in these germinal centers by follicular dendritic cells (FDCs), accompanied by disregulation in interactions among FDCs, B-cells and follicular Th cells, reduced elimination of autoreactive B-cells and subsequent, antigen-driven, generation of affinity-mature long-lived plasma cells and memory B-cells producing high affinity IgG autoantibodies, which can result in the development of autoimmune and inflammatory disorders. See, e.g., Vinuesa et al., Immunology 9:845-857 (2009). Furthermore, over-expression of CXCR5 receptor in certain cancers has been reported to promote CXCL13-dependent cell proliferation and metastasis.

High-level expression of CXCL13 (BCA-1) and its receptor, CXCR5, has been observed in H. pylori-induced gastric lymphoid follicles and mucosa-associated lymphoid tissue (MALT) lymphomas. See, e.g., Mazzucchelli et al., J Clin Invest 104:R49-R54 (1999). Furthermore, CXCL13 (BCA-1) expression was found in all samples of H. pylori-induced gastritis. Id. In the gastric mucosa of H. heilmannii-infected wild-type mice, the mRNA expression level of CXCL13, which is known to be involved in organogenesis of lymphatic tissues (including MALT), was significantly higher than that of uninfected mice. See Nobutani et al., FEMS Immunol Med Microbiol 60:156-164 (2010).

The need for therapies that target CXCL13-mediated signaling pathways has become increasingly apparent in the recent years. The mechanisms of action for such treatments would include, e.g., blockade of CXCL13 interaction with its receptor resulting in interference with B cell and follicular B-helper T cell migration into inflamed tissues and germinal center formation (e.g., in the case of autoimmune disease) and inhibition of cancer cell proliferation and its ability to spread in oncological disorders.

### BRIEF SUMMARY OF THE INVENTION

The invention relates to binding agents, such as antibodies and antigen binding fragments thereof, that specifically bind to CXCL13.

One aspect of the invention relates to an isolated antigen binding molecule which specifically binds to the same CXCL13 epitope as a reference monoclonal antibody or competitively inhibits a reference monoclonal antibody, wherein the reference monoclonal antibody is selected from the group consisting of MAb 5261, MAb 5378, MAb 5080, MAb 1476, 3D2, and 3C9, or a variant thereof.

In another embodiment, the antibody or fragment thereof specifically binds CXCL13 and binds to an epitope sequence within SEQ ID NO: 26 or a variant thereof.

In certain embodiments, the isolated antibody or antigen-binding fragment thereof specifically binds CXCL13 and binds to the same CXCL13 epitope as a reference monoclonal antibody or fragment thereof or competitively inhibits a reference monoclonal antibody or fragment thereof, wherein the reference monoclonal antibody or fragment thereof comprises a heavy chain variable region (VH) comprising an amino acid sequence selected from the group consisting of SEQ ID NO: 13 and SEQ ID NO: 3 or a variant thereof. In another embodiment, the light chain variable region (VL) of the reference antibody or fragment thereof comprises an amino acid sequence selected from the group consisting of SEQ ID NO: 15, SEQ ID NO: 17, and SEQ ID NO: 8 or a variant thereof.

In one embodiment, the isolated antibody or antigen-binding fragment thereof specifically binds CXCL13 and binds to the same CXCL13 epitope as a reference monoclonal antibody or fragment thereof or competitively inhibits a reference monoclonal antibody or fragment thereof, wherein the reference monoclonal antibody or fragment thereof comprises a Chothia-Kabat heavy chain complementarity determining region-1 (VH-CDR1) amino acid sequence identical to SEQ ID NO: 4; a Kabat heavy chain complementarity determining region-2 (VH-CDR2) amino acid sequence identical to SEQ ID NO: 5; a Kabat heavy chain complementarity determining region-3 (VH-CDR3) amino acid sequence identical to SEQ ID NO: 6; a Kabat light chain complementarity determining region-1 (VL-CDR1) amino acid sequence identical to SEQ ID NO: 16 or 9; a Kabat light chain complementarity determining region-2 (VL-CDR2) amino acid sequence identical to SEQ ID NO: 10; and a Kabat light chain complementarity determining region-3 (VL-CDR3) amino acid sequence identical to SEQ ID NO: 11.

A further aspect of the invention is directed to an isolated polypeptide or an isolated polynucleotide encoding the same, wherein the polypeptide consists of an epitope for binding an anti-CXCL13 antibody, wherein the epitope comprises SEQ ID NO: 26 or a variant thereof.

Another aspect of the invention is directed to methods for neutralizing CXCL13 in an animal, comprising administering to said animal a composition comprising an isolated antibody or fragment thereof or a composition of the invention.

### BRIEF DESCRIPTION OF THE DRAWINGS/FIGURES

FIG. 1. Specificity ELISA results showing the binding of mouse anti-human CXCL13 antibodies (3D2 and 3C9) to recombinant human CXCL13 (1A), recombinant mouse CXCL13 (1B), and recombinant cynomolgus monkey CXCL13 (1C) compared to antibody controls (mouse MAb 801 and/or rat MAb 470). EC50 values are shown and were obtained with four-parameter sigmoidal curve fit (curves are shown on the graph; the R² for the curves that produced EC50 values was 0.99).

FIG. 2. Epitope Competition ELISA results showing the percent inhibition of biotinylated 3D2 binding to human CXCL13 for mouse anti-human CXCL13 antibodies (3C9 and 3D2) compared to results with no competitor or MAb 801.

FIG. 3. Capture Epitope Competition ELISA results showing 3D2 inhibition of biotin-3C9 binding to native or recombinant human CXCL13 (3A) and biotin-3D2 binding to native or recombinant mouse CXCL13 (3B). Curves were fitted using four-parameter sigmoidal curve fit (curves are shown on the graph; the R²=0.99). The differences in EC50 values were analyzed by unpaired t-test and were found to be P>0.05.

FIG. 4. B-cell migration results showing the effect of 3D2 and 3C9 on human CXCL13 induced migration of human pre-B-697-hCXCR5 cells (4A) and human SDF-1 alpha induced migration of pre-B-697-hCXCR4 cells (4B). Mouse IgG was used as a negative control. MAb 801 was used as a positive control for inhibition of human CXCL13 migration, and MAb 87A was used as a positive control for inhibition of human SDF-1 alpha-induced migration.

FIG. 5. Percent inhibition of splenocyte migration in C57Black/6 by 3D2, MAb 470, Mouse IgG (control), or Rat IgG (control) (5A) and in SJL/J by 3D2, 3C9, MAb 470, or Mouse IgG (control) (5B). The results are presented as mean of two (C57Black/6 migration) independent experiments +/−SD and three (SJL/J migration) independent experiments +/−SEM. A comparison of the effect of 3D2 on C57Black/6 and SJL/J migration (5C) was analyzed by unpaired t-test which produced P value >0.05. Curves were fitted using four-parameter sigmoidal curve fit (curves are shown on the graph; R²=0.99).

FIG. 6. CXCL13-mediated endocytosis results for human CXCL13-mediated endocytosis (6A) and mouse CXCL13-mediated endocytosis (6B) of human and mouse CXCR5 receptors by 3D2 or controls (MAb 470 and/or Mouse IgG). A comparison of human and mouse CXCL13-mediated endocytosis EC50 values was calculated from sigmoidal dose response curves with R² values equal to 1 (mouse endocytosis) and 0.994 (human endocytosis) is shown (6C). The data comparing 3D2 effect on human and mouse receptor endocytosis was analyzed by unpaired t-test which produced P value >0.05.

FIG. 7. EAE disease progression in mice treated with 3D2 (start at Day 0), 3D2 (start at Score>1), or Mouse IgG control (RR-EAE-1 Study). Each data point represents a mean of scores taken from 9 mice. Group means (GMS) were compared by using one-way ANOVA followed by Bonferroni's multiple comparison post test.

FIG. 8. EAE disease progression in mice treated with 3D2 (start at Day 0), 3D2 (start at Day 6), 3D2 (start at Day >2), or Mouse IgG control (RR-EAE-2 Study). Each data point represents a mean of scores taken from 9 mice. Group means (GMS) were compared by using one-way ANOVA followed by Bonferroni's multiple comparison post test.

FIG. 9. Kidney pathology in mice with advanced lupus nephritis after 3D2 or Mouse IgG (control) treatment (SLE-1 Study). For proteinurea scores (9A) and kidney pathology scores for Glomerulonephritis, Interstitial nephritis, and Vasculitis (9B), each data point represents mean of ten measurements.

FIG. 10. Kidney pathology in mice with early lupus disease after 3D2 and Mouse IgG (control) treatment (SLE-2 Study). For proteinurea scores (10A) and kidney pathology scores for Glomerulonephritis and Interstitial nephritis (10B) each data point represents 7 mice from 3D2-treated group and 9 mice from mouse IgG-treated group.

FIG. 11: Histology sections showing the effect of 3D2 on the number of germinal centers (GCs) and primary follicles in lupus mouse spleen. Spleen sections were stained with GL-7 (GC stain), B220 antibody (B cell marker), or antibody against follicular dendritic cells (FDCs) from 3D2-treated (11A) and mouse IgG-treated (11B) NZB/NZWF1 mice.

FIG. 12. Primary follicles and GC size in spleen of lupus mice treated with 3D2. Values are shown as mean+/−SEM with 5 mice per group. Mice treated with 3D2 (“tx”) showed a trend towards decreased numbers of GCs when expressed as a ratio of primary: secondary (GC) follicles (p=0.19) (12A) and a significant decrease in GC size (p=0.03) (12B).

FIG. 13. Polynucleotide and amino acid sequences of 3D2 Variable Heavy Chain (H1609) and Variable Light Chain (L0293). Complementarity determining regions (CDRs) are underlined.

FIG. 14 Amino acid sequences for humanization of chimeric 3D2 showing the modification of Variable Heavy Chain H1609 to H2177 (14A) and Variable Light Chain L0293 to L5055 to L5140 (14B). The putative glycosylation site and complementarity determining regions (CDRs) are boxed.

FIG. 15. Polynucleotide and amino acid sequences of MAb 5261 Variable Heavy and Light Chains (H2177/L5140) and MAb 5080 Variable Heavy and Light Chains (H2177/L5055). Complementarity determining regions (CDR) are underlined.

FIG. 16. Specificity ELISA results for MAb 5261, MAb 5080, MAb 1476, and Human Isotype Control binding to recombinant human (16A), cynomolgus monkey (16B) and mouse (16C) CXCL13. Each data point represents mean of triplicate measurements. EC50 values were calculated from four-parameter sigmoidal curve fit (curves are shown on the graph; R² for the curves that produced EC50 values were 0.99). NB=no binding.

FIG. 17. Capture Epitope Competition ELISA results for MAb 5261, MAb 5080, and 3D2 binding to native human (17A) and native mouse (17B) CXCL13. Each data point represents an average of duplicate measurements from one of at least three independent experiments. Curves were fitted using four-parameter sigmoidal curve fit (curves are shown on the graph; R²=0.99).

FIG. 18. Percent Inhibition of human pre-B-697-hCXCR5 (18A) and human tonsillar cell (18B) migration by MAb 5261. Data represent an average of triplicate measurements +/−SEM from one of at least three experiments. Curves were fitted using four-parameter sigmoidal curve fit (curves are shown on the graph; R²=0.98-0.99).

FIG. 19. Percent Inhibition of SJL/J (19A) and C57Black/6 (19B) Splenocyte Migration by MAb 5261. Data from representative experiments are shown as mean of duplicate measurements +/−SD.

FIG. 20. Percent Inhibition of human CXCL13-mediated internalization of human CXCR5 receptor by MAb 5261 and Isotype Control. Data are average of triplicate measurements from one of at least three independent experiments. Curve was fitted using four-parameter sigmoidal curve fit (curves are shown on the graph; R²=0.99).

FIG. 21. Polynucleotide and amino acid sequence of MAb 5378 Variable Heavy Chain (H5188) and Variable Light Chain (L5153). Complementarity determining regions (CDRs) are underlined.

FIG. 22. Epitope Competition ELISA results for MAb 5378, MAb 5261, and MAb 470.

FIG. 23. Specificity ELISA results for MAb 5378, 3D2, and Mouse Isotype Control binding to recombinant human (23A), cynomolgus monkey (23B) and mouse (23C) CXCL13. Each data point represents mean of triplicate measurements. EC50 values were calculated from four-parameter sigmoidal curve fit (curves are shown on the graph; R² for the curves that produced EC50 values were 0.99).

FIG. 24. Percent Inhibition of human pre-B-697-hCXCR5 (24A), human tonsillar cells (24B) and C57Black6 mouse spleenocyte (24C) migration by MAb 5261 or MAb 5378 (24A-B) and MAb 5378 or 3D2 (24C). Data represent an average of triplicate measurements +/−SEM from one of at least three experiments. Curves were fitted using four-parameter sigmoidal curve fit (curves are shown on the graph; R²=0.99).

FIG. 25. Percent Inhibition of human CXCL13-mediated internalization of human CXCR5 receptor by MAb 5378, MAb 5261, 3D2, Mouse Isotype Control, or Human Isotype Control. Data points for 5261 and 5378 represent average of measurements from two independent experiments. Data points for 3D2 and Isotype Controls represent average of triplicate measurements from a single experiment. Curve was fitted using four-parameter sigmoidal curve fit (curves are shown on the graph; R²=0.99). NE=no effect.

FIG. 26. Collagen-induced arthritis (CIA) disease progression in mice treated with MAb 5376, etanercept, or Mouse IgG (control) (CIA-1 Study). Each data point represents a mean of scores taken from 10 mice. Group means were compared by using one-way ANOVA followed by Bonferroni's multiple comparison post test.

FIG. 27. Collagen-induced arthritis (CIA) disease progression in mice treated with MAb 5378, etanercept, MAb 470, or Mouse IgG (control) (CIA-2 Study). Each data point represents a mean of scores taken from 10 mice. Group means were compared by using one-way ANOVA followed by Bonferroni's multiple comparison post test.

FIG. 28. Germinal center formation in NP-CGG immunized mice treated with MAb 5378, Mouse Isotype Control, or no treatment (GC-1 Study). Each spleen data point represents a mean of values measured from three mice. Each lymph node data point represents a single value obtained from pooled cells collected from three mice.

FIG. 29. Treatment schedule for H. heilmannii infection of mice and antibody administration.

FIG. 30. H. heilmannii specific 16s rRNA genes were amplified in all gastric samples obtained from H. heilmannii infected mice including isotype control antibody treatment and anti-CXCL13 antibody treatment. Positive control (P) and negative control (N) are also shown.

FIG. 31. The mRNA expression level of CXCL13 in the gastric mucosa of H. heilmannii (HH) infected wild-type (WT) mice 1 month (31A) and 3 months (31B) after infection as determined by real-time quantitative PCR (values are normalized to mouse beta-actin expression levels in each sample).

FIG. 32. The expression of CXCL13 mRNA and β-actin in the stomach of H. heilmannii infected mice after isotype control antibody or anti-CXCL13 antibody treatment (upper panel). The expression of CXCL13 mRNA and β-actin in the stomach of noninfected mice (lower panel). Positive control (P) and negative control (N) are also shown.

FIG. 33. Hematoxylin and eosin (H&E) stained stomach samples from isotype control antibody treated mouse (upper left panel) and anti-CXCL13 antibody treated mouse (upper right panel) three months after H. heilmannii infection. The lower panel shows the number of gastric lymphoid follicles identified in stomach samples from isotype control antibody and anti-CXCL13 antibody treated mice.

### DETAILED DESCRIPTION OF THE INVENTION

In one embodiment, the present invention relates to binding agents, such as antibodies and antigen binding fragments thereof, that specifically bind to CXCL13 and epitopes recognized by such binding agents. In some embodiments the presently disclosed anti-CXCL13 antibodies or antigen-binding fragments thereof recognize the epitope of anti-CXCL13 antibody MAb 5261. In some embodiments, the epitope sequence comprises the amino acid sequence set forth in SEQ ID NO: 26 or a variant thereof. CXCL13 binding agents find use in methods for neutralizing CXCL13, for treating autoimmune or inflammatory diseases, cancers, mucosa-associated lymphoid tissue (MALT) lymphomas, gastric or duodenal ulcers, and inhibiting gastric lymphoid follicles.

The invention may also encompass isolated polypeptides and isolated polynucleotides encoding the same, wherein the polypeptide consists of an epitope for binding an anti-CXCL13 antibody of the invention. These polypeptides correspond to a portion of the CXCL13 antigen that binds to antibody MAb 5261. For example, in one embodiment, the isolated polypeptide consists of an epitope for binding an anti-CXCL13 antibody that comprises the sequence set forth in SEQ ID NO: 26 or a variant thereof. Such polypeptides find use in methods for producing or detecting antibodies that selectively bind to CXCL13. The ability of a polypeptide to be used in the production or detection of antibodies is referred to herein as “antigenic activity” or “immunogenic activity”.

In accordance with some embodiments, the invention may also encompass variants and fragments of the sequence set forth in SEQ ID NO: 26 that retain the antigenic activity of the original polypeptide, and polynucleotides that encode such polypeptides.

In accordance with some embodiments, the invention may also encompass cancer peptide vaccines that incorporate CXCL13 peptides in a pharmaceutical composition or as a fusion protein to stimulate a strong and effective antibody and/or cellular immune responses to CXCL13. Specifically, the invention may encompass peptide vaccines comprised of the sequence set forth in SEQ ID NO: 26 or a variant thereof. The present invention may also relate to pharmaceutical compositions comprising the peptide vaccines. The vaccines may be used for the treatment of cancers which express target proteins, such as CXCL13. These vaccines are likely to induce a strong, comprehensive immune response against the target proteins, and thereby induce an immune reaction against tumors expressing such target proteins.

As used herein, the terms “CXCL13” and “CXCL13 polypeptide” are used interchangeably. In certain embodiments, CXCL13 may include a full-sized CXCL13 or a fragment thereof, or a CXCL13 variant polypeptide, wherein the fragment of CXCL13 or CXCL13 variant polypeptide retains some or all functional properties of the full-sized CXCL13. The human CXCL13 polypeptide and polynucleotide sequences (SEQ ID NOs: 19 and 20, respectively) have been described, see, e.g., Legler, et. al., J. Exp. Med. 187(4):655-660 (1998). The mouse CXCL13 polypeptide and polynucleotide sequences (SEQ ID NOs: 21 and 22, respectively) have been described, see, e.g., Gunn, et. al., Nature 391(6669):799-803 (1998). Furthermore, the cynomolgus monkey CXCL13 polypeptide sequence has been described as shown in SEQ ID NO: 23.

A “binding molecule” or “antigen binding molecule” refers in its broadest sense to a molecule that specifically binds an antigenic determinant. In one embodiment, the binding molecule specifically binds to CXCL13 (also called BCA-1). In another embodiment, a binding molecule useful in the presently disclosed methods is an antibody or an antigen binding fragment thereof, e.g., an anti-CXCL13 antibody. In another embodiment, a binding molecule comprises at least one heavy or light chain CDR of an antibody molecule. In another embodiment, a binding molecule comprises at least two CDRs from one or more antibody molecules. In another embodiment, a binding molecule comprises at least three CDRs from one or more antibody molecules. In another embodiment, a binding molecule comprises at least four CDRs from one or more antibody molecules. In another embodiment, a binding molecule comprises at least five CDRs from one or more antibody molecules. In another embodiment, a binding molecule comprises at least six CDRs from one or more antibody molecules. In certain embodiments, one or more of the CDRs is from MAb 5261, MAb 5378, MAb 5080, MAb 1476, or 3D2.

In some embodiments, the invention involves certain anti-CXCL13 antibodies, or antigen-binding fragments, variants, or derivatives thereof. Unless specifically referring to full-sized antibodies such as naturally occurring antibodies, the term “anti-CXCL13 antibodies” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al. (1988) Antibodies: A Laboratory Manual (2nd ed.; Cold Spring Harbor Laboratory Press).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgD, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention. The use of all immunoglobulin classes are clearly within the scope of the presently disclosed methods, however, the following discussion will generally be directed to the IgG class of immunoglobulin molecules. With regard to IgG, a standard immunoglobulin molecule comprises two identical light chain polypeptides of a molecular weight of approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B-cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL or VK) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated herein, the variable region allows the antibody to selectively recognize and specifically bind epitopes on antigens. That is, the VL domain and VH domain, or subset of the complementarity determining regions (CDRs) within these variable domains, of an antibody combine to form the variable region that defines a three dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable domain by one of ordinary skill in the art, since they have been precisely defined (see below).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al. (1983) U.S. Dept. of Health and Human Services, “Sequences of Proteins of Immunological Interest” and by Chothia and Lesk, J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table 1 as a comparison. The exact residue numbers that encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al. (1983) U.S. Dept. of Health and Human Services, “Sequence of Proteins of Immunological Interest.” Unless otherwise specified, references to the numbering of specific amino acid residue positions in an anti-CXCL13 antibody or antigen-binding fragment, variant, or derivative thereof of the present invention are according to the Kabat numbering system.

Antibodies or antigen-binding fragments, variants, or derivatives thereof of the invention include, but are not limited to, polyclonal, monoclonal, multispecific, human, humanized, primatized, or chimeric antibodies, single-chain antibodies, epitope-binding fragments, e.g., Fab, Fab′ and F(ab′)2, Fd, Fvs, single-chain Fvs (scFv), disulfide-linked Fvs (sdFv), fragments comprising either a VL or VH domain, fragments produced by a Fab expression library, and anti-idiotypic (anti-Id) antibodies (including, e.g., anti-Id antibodies to anti-CXCL13 antibodies). ScFv molecules are known in the art and are described, e.g., in U.S. Pat. No. 5,892,019 Immunoglobulin or antibody molecules of the invention can be of any type (e.g., IgG, IgE, IgM, IgD, IgA, and IgY), class (e.g., IgG1, IgG2, IgG3, IgG4, IgA1, and IgA2, etc.), or subclass of immunoglobulin molecule.

As used herein, the term “heavy chain portion” includes amino acid sequences derived from an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. For example, a binding polypeptide for use in the invention may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a binding polypeptide of the invention comprises a polypeptide chain comprising a CH3 domain. Further, a binding polypeptide of the invention may lack at least a portion of a CH2 domain (e.g., all or part of a CH2 domain) As set forth above, it will be understood by one of ordinary skill in the art that these domains (e.g., the heavy chain portions) may be modified such that they vary in amino acid sequence from the naturally occurring immunoglobulin molecule.

In certain anti-CXCL13 antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers useful in the presently disclosed methods are not identical. For example, each monomer may comprise a different target binding site, forming, for example, a bispecific antibody.

The heavy chain portions of a binding molecule for use in the methods disclosed herein may be derived from different immunoglobulin molecules. For example, a heavy chain portion of a polypeptide may comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain, e.g., a kappa or lambda light chain. Preferably, the light chain portion comprises at least one of a VL or CL domain.

Anti-CXCL13 antibodies, or antigen-binding fragments, variants, or derivatives thereof disclosed herein may be described or specified in terms of the epitope(s) or portion(s) of an antigen, e.g., a target polypeptide disclosed herein (e.g., CXCL13) that they recognize or specifically bind. The portion of a target polypeptide that specifically interacts with the antigen binding domain of an antibody is an “epitope,” or an “antigenic determinant.” A target polypeptide may comprise a single epitope, but typically comprises at least two epitopes, and can include any number of epitopes, depending on the size, conformation, and type of antigen. Furthermore, it should be noted that an “epitope” on a target polypeptide may be or may include non-polypeptide elements, e.g., an epitope may include a carbohydrate side chain.

The minimum size of a peptide or polypeptide epitope for an antibody is thought to be about four to five amino acids. Peptide or polypeptide epitopes may contain at least six, at least seven, at least eight, at least nine, at least ten, at least eleven, at least twelve, at least thirteen, at least fourteen, at least 15, and in some embodiments, between at least about 15 to about 30 amino acids. Since a CDR can recognize an antigenic peptide or polypeptide in its tertiary form, the amino acids comprising an epitope need not be contiguous, and in some cases, may not even be on the same peptide chain. In some embodiments, the epitope is conformational. A peptide or polypeptide epitope recognized by anti-CXCL13 antibodies of the invention may contain a sequence of at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or between about 15 to about 30 contiguous or non-contiguous amino acids of CXCL13.

In certain embodiments, the anti-CXCL13 antibodies of the invention bind to the same epitope as a reference monoclonal antibody (e.g., mAb 5261, 5378, 5080, 1476, or 3D2).

In some embodiments, the anti-CXCL13 antibodies of the invention recognize an epitope comprising the sequence set forth in SEQ ID NO: 26 or a variant or fragment thereof. In some of these embodiments, the anti-CXCL13 antibody binds to an epitope comprising at least 5, at least 6, at least 7, at least 8, at least 9, at least 10, at least 11, at least 12, at least 13, or at least 14 contiguous amino acid residues of SEQ ID NO: 26. In other embodiments, the anti-CXCL13 antibody binds to an epitope comprising, consisting essentially of, or consisting of an amino acid sequence having at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or greater identity to SEQ ID NO: 26. In still other embodiments, the anti-CXCL13 antibody binds to an epitope having a sequence identical to SEQ ID NO: 26, except for 10, 9, 8, 7, 6, 5, 4, 3, 2, or 1 amino acid substitutions, which can be conservative amino acid substitutions. In certain embodiments, the anti-CXCL13 antibody binds to an epitope having a sequence identical to SEQ ID NO: 26, except for 4 or fewer amino acid substitutions.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody that “preferentially binds” to a given epitope would more likely bind to that epitope than to a related epitope, even though such an antibody may cross-react with the related epitope.

By way of non-limiting example, an antibody may be considered to bind a first epitope preferentially if it binds said first epitope with a dissociation constant (K_{D}) that is less than the antibody's K_{D} for the second epitope. In another non-limiting example, an antibody may be considered to bind a first antigen preferentially if it binds the first epitope with an K_{D} that is at least one order of magnitude less than the antibody's K_{D} for the second epitope. In another non-limiting example, an antibody may be considered to bind a first epitope preferentially if it binds the first epitope with an K_{D} that is at least two orders of magnitude less than the antibody's K_{D} for the second epitope.

In another non-limiting example, an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an k(off) that is at least one order of magnitude less than the antibody's k(off) for the second epitope. In another non-limiting example, an antibody may be considered to bind a first epitope preferentially if it binds the first epitope with an k(off) that is at least two orders of magnitude less than the antibody's k(off) for the second epitope. An antibody or antigen-binding fragment, variant, or derivative useful in the methods disclosed herein may be said to bind a target polypeptide disclosed herein (e.g., CXCL13, e.g., human, murine, or both human and murine CXCL13) or a fragment or variant thereof with an off rate (k(off)) of less than or equal to 5×10⁻² sec⁻¹, 10⁻² sec⁻¹, or 5×10⁻³ sec⁻¹. In certain embodiments, the k(off) is less than or equal to about 3×10⁻², e.g., wherein the CXCL13 is human or mouse. In another embodiment, the k(off) is less than or equal to about 3×10⁻³, e.g., wherein the CXCL13 is human or mouse. In another embodiment, the k(off) is less than or equal to about 4×10⁻³, e.g., wherein the CXCL13 is human or mouse. In one embodiment, an antibody useful in the presently disclosed methods may be said to bind a target polypeptide disclosed herein (e.g., CXCL13, e.g., human, murine, or both human and murine CXCL13) or a fragment or variant thereof with an off rate (k(off)) less than or equal to 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹.

An antibody or antigen-binding fragment, variant, or derivative of the invention may be said to bind a target polypeptide disclosed herein (e.g., CXCL13, e.g., human, murine, or both human and murine CXCL13) or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶M⁻¹ sec⁻¹ or 5×10⁶ M⁻¹ sec⁻¹. In certain embodiments, the k(on) is greater than or equal to about 5×10⁵, e.g., wherein the CXCL13 is human; or the k(on) is greater than or equal to about 1×10⁵, e.g., wherein the CXCL13 is mouse. In another embodiment, the k(on) is greater than or equal to about 1×10⁶, e.g., wherein the CXCL13 is human or mouse. In another embodiment, the k(on) is greater than or equal to about 1×10⁶, e.g., wherein the CXCL13 is human or mouse. In one embodiment, an antibody of the invention may be said to bind a target polypeptide disclosed herein (e.g., CXCL13, e.g., human, murine, or both human and murine CXCL13) or a fragment or variant thereof with an on rate (k(on)) greater than or equal to 10⁵ M⁻¹ sec⁻¹, 5×10⁵ M⁻¹ sec⁻¹, 10⁶M⁻¹ sec⁻¹, or 5×10⁶ M⁻¹ sec⁻¹ or 10⁷ M⁻¹ sec⁻¹.

An antibody is said to competitively inhibit binding of a reference antibody or antigen-binding fragment thereof, e.g., an anti-CXCL13 antibody, to a given epitope if it preferentially binds to that epitope to the extent that it blocks, to some degree, binding of the reference antibody or antigen-binding fragment thereof to the epitope. Competitive inhibition may be determined by any method known in the art, for example, competition ELISA assays. An antibody or antigen-binding fragment thereof may be said to competitively inhibit binding of the reference antibody or antigen-binding fragment thereof to a given epitope by at least 90%, at least 80%, at least 70%, at least 60%, or at least 50%.

In certain embodiments, the anti-CXCL13 antibodies of the invention competitively inhibit a reference monoclonal antibody (e.g., mAb 5261, 5378, 5080, 1476, or 3D2) from binding to CXCL13.

As used herein, a “reference antibody” or “reference antigen-binding fragment or an antibody” refers to any antibody or antigen-binding fragment thereof with which an epitope or binding specificity is being compared or competed. In some embodiments, the anti-CXCL13 antibodies of the invention bind to the same epitope as or competitively inhibit a reference monoclonal antibody. In some of these embodiments, the reference antibody can be selected from the group consisting of mAb 5261, 5378, 5080, 1476, and 3D2. In other embodiments, the reference antibody comprises, consists essentially of, or consists of an immunoglobulin heavy chain variable domain (VH domain), where at least one of the CDRs of the VH domain has an amino acid sequence that is at least about 80%, about 85%, about 90%, about 95%, about 96%, about 97%, about 98%, about 99%, or identical to CDR1, CDR2 or CDR3 of SEQ ID NO: 3 or 13.

In another embodiment, the reference antibody or antigen-binding fragment thereof comprises, consists essentially of, or consists of an immunoglobulin heavy chain variable domain (VH domain), where at least one of the CDRs of the VH domain has an amino acid sequence that is at least about 80%, about 85%, about 90%, about 95%, about 96%, about 97%, about 98%, about 99%, or identical to SEQ ID NO: 4, 5, or 6.

In another embodiment, the reference antibody or antigen-binding fragment thereof comprises, consists essentially of, or consists of an immunoglobulin heavy chain variable domain (VH domain), where the VH domain has an amino acid sequence that is at least about 80%, about 85%, about 90%, about 95%, about 96%, about 97%, about 98%, about 99%, or identical to SEQ ID NO: 3 or 13.

In another embodiment, the reference antibody or antigen-binding fragment thereof comprises, consists essentially of, or consists of an immunoglobulin heavy chain variable domain (VH domain), where at least one of the CDRs of the VH domain has an amino acid sequence identical, except for 1, 2, 3, 4, or 5 conservative amino acid substitutions, to CDR1, CDR2 or CDR3 of SEQ ID NO: 3 or 13.

In another embodiment, the reference antibody or antigen-binding fragment thereof comprises, consists essentially of, or consists of an immunoglobulin heavy chain variable domain (VH domain), where at least one of the CDRs of the VH domain has an amino acid sequence identical, except for 1, 2, 3, 4, or 5 conservative amino acid substitutions, to SEQ ID NO: 4, 5, or 6.

In another embodiment, the reference antibody or antigen-binding fragment thereof comprises, consists essentially of, or consists of a VH domain that has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 3 or 13, wherein an anti-CXCL13 antibody comprising the encoded VH domain specifically or preferentially binds to CXCL13.

In another embodiment, the reference antibody or antigen-binding fragment thereof comprises, consists essentially of, or consists of an immunoglobulin light chain variable domain (VL domain), where at least one of the CDRs of the VL domain has an amino acid sequence that is at least about 80%, about 85%, about 90%, about 95%, about 96%, about 97%, about 98%, about 99%, or identical to CDR1, CDR2 or CDR3 of SEQ ID NO: 8, 15, or 17.

In another embodiment, the reference antibody or antigen-binding fragment thereof comprises, consists essentially of, or consists of an immunoglobulin light chain variable domain (VL domain), where at least one of the CDRs of the VL domain has an amino acid sequence that is at least about 80%, about 85%, about 90%, about 95%, about 96%, about 97%, about 98%, about 99%, or identical to SEQ ID NO: 9, 16, 10, or 11.

In another embodiment, the reference antibody or antigen-binding fragment thereof comprises, consists essentially of, or consists of an immunoglobulin light chain variable domain (VL domain), where the VL domain has an amino acid sequence that is at least about 80%, about 85%, about 90%, about 95%, about 96%, about 97%, about 98%, about 99%, or identical to SEQ ID NO: 8, 15, or 17.

In another embodiment, the reference antibody or antigen-binding fragment thereof comprises, consists essentially of, or consists of an immunoglobulin light chain variable domain (VL domain), where at least one of the CDRs of the VL domain has an amino acid sequence identical, except for 1, 2, 3, 4, or 5 conservative amino acid substitutions, to CDR1, CDR2 or CDR3 of SEQ ID NO: 8, 15, or 17.

In another embodiment, the reference antibody or antigen-binding fragment thereof comprises, consists essentially of, or consists of an immunoglobulin light chain variable domain (VL domain), where at least one of the CDRs of the VL domain has an amino acid sequence identical, except for 1, 2, 3, 4, or 5 conservative amino acid substitutions, to SEQ ID NO: 9, 16, 10, or 11.

In a further embodiment, the reference antibody or antigen-binding fragment thereof comprises, consists essentially of, or consists of a VL domain that has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 8, 15, or 17, wherein an anti-CXCL13 antibody comprising the encoded VL domain specifically or preferentially binds to CXCL13.

In a still further embodiment, the reference antibody or antigen-binding fragment thereof comprises, consists essentially of, or consists of a VH domain that has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 3 or 13, and a VL domain that has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 8, 15, or 17.

In certain embodiments, the reference antibody or antigen-binding fragment thereof comprises, consists essentially of, or consists of a VH domain that has the amino acid sequence set forth in SEQ ID NO: 13, and a VL domain that has the amino acid sequence set forth in SEQ ID NO: 15.

In certain embodiments, the reference antibody or antigen-binding fragment thereof comprises, consists essentially of, or consists of a VH domain that has the amino acid sequence set forth in SEQ ID NO: 13, and a VL domain that has the amino acid sequence set forth in SEQ ID NO: 17.

In certain embodiments, the reference antibody or antigen-binding fragment thereof comprises, consists essentially of, or consists of a VH domain that has the amino acid sequence set forth in SEQ ID NO: 3, and a VL domain that has the amino acid sequence set forth in SEQ ID NO: 8.

In a further embodiment, the reference antibody or antigen-binding fragment thereof comprises, consists essentially of, or consists of a VH domain comprising a VH-CDR1 having the amino acid sequence set forth in SEQ ID NO: 4, a VH-CDR2 having the amino acid sequence set forth in SEQ ID NO: 5, and a VH-CDR3 having the amino acid sequence set forth in SEQ ID NO: 6, and a VL domain comprising a VL-CDR1 having the amino acid sequence set forth in SEQ ID NO: 16 or 9, a VL-CDR2 having the amino acid sequence set forth in SEQ ID NO: 10, and a VL-CDR3 having the amino acid sequence set forth in SEQ ID NO: 11.

As used herein, the term “affinity” refers to a measure of the strength of the binding of an individual epitope with the CDR of an immunoglobulin molecule. See, e.g., Harlow et al. (1988) Antibodies: A Laboratory Manual (Cold Spring Harbor Laboratory Press, 2nd ed.) pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Anti-CXCL13 antibodies or antigen-binding fragments, variants, or derivatives thereof of the invention may also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

Anti-CXCL13 binding molecules, e.g., antibodies or antigen-binding fragments, variants or derivatives thereof, of the invention may also be described or specified in terms of their binding affinity to a polypeptide, e.g., CXCL13, e.g., human, murine, or both human and murine CXCL13. In certain embodiments, the binding affinities of the antibodies or antigen-binding fragments thereof useful in the presently disclosed methods include those with a dissociation constant or Kd less than or no greater than 5×10⁻²M, 10⁻²M, 5×10⁻³ M, 10⁻³M, 5×10⁻⁴M, 10⁻⁴ M, 5×10⁻⁵M, 10⁻⁵ M, 5×10⁻⁶M, 10⁻⁶M, 5×10⁻⁷M, 10⁻⁷ M, 5×10⁻⁸M, 10⁻⁸M, 5×10⁻⁹M, 10⁻⁹M, 5×10⁻¹⁰ M, 10⁻¹⁰ M, 5×10⁻¹¹ M, 10⁻¹¹ M, 5×10⁻¹²M, 10⁻¹²M, 5×10⁻¹³ M, 10⁻¹³ M, 5×10⁻¹⁴ M, 10⁻¹⁴M, 5×10⁻¹⁵ M, or 10⁻¹⁵ M. In one embodiment, the anti-CXCL13 binding molecule, e.g., an antibody or antigen binding fragment thereof, of the invention binds human CXCL13 with a Kd of less than about 5×10⁻⁹ M to about 5×10⁻¹° M, e.g. the Kd is less than or equal to about 5×10⁻⁹M. In another embodiment, the anti-CXCL13 binding molecule, e.g., an antibody or antigen binding fragment thereof, binds murine CXCL13 with a Kd of less than about 5×10⁻⁷M to about 9×10⁻⁹M, e.g., the Kd is less than or equal to about 8×10⁻⁹M.

Anti-CXCL13 antibodies or antigen-binding fragments, variants or derivatives thereof of the invention may be “multispecific,” e.g., bispecific, trispecific, or of greater multispecificity, meaning that it recognizes and binds to two or more different epitopes present on one or more different antigens (e.g., proteins) at the same time. Thus, whether an anti-CXCL13 antibody is “monospecific” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains present in a binding polypeptide or CXCL13 binding molecule, e.g., an antibody or antigen binding fragment thereof. Each binding domain specifically binds one epitope. When a binding polypeptide or CXCL13 binding molecule comprises more than one binding domain, each binding domain may specifically bind the same epitope, for an antibody with two binding domains, termed “bivalent monospecific,” or to different epitopes, for an antibody with two binding domains, termed “bivalent bispecific.” An antibody or antigen binding fragment thereof may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Patent Appl. Publ. Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148: 1547-1553 (1992).

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat E A et al.). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, “human” or “fully human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example, in U.S. Pat. No. 5,939,598, which is herein incorporated by reference in its entirety. “Human” or “fully human” antibodies also include antibodies comprising at least the variable domain of a heavy chain, or at least the variable domains of a heavy chain and a light chain, where the variable domain(s) have the amino acid sequence of human immunoglobulin variable domain(s).

“Human” or “fully human” antibodies also include “human” or “fully human” antibodies, as described above, that comprise, consist essentially of, or consist of, variants (including derivatives) of known anti-CXCL13 molecules (e.g., the VH regions and/or VL regions), which antibodies or fragments thereof immunospecifically bind to a CXCL13 polypeptide or fragment or variant thereof. Standard techniques known to those of skill in the art can be used to introduce mutations in the nucleotide sequence encoding a human anti-CXCL13 antibody, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3.

In certain embodiments, the amino acid substitutions are conservative amino acid substitutions, discussed further below. Alternatively, mutations can be introduced randomly along all or part of the coding sequence, such as by saturation mutagenesis, and the resultant mutants can be screened for biological activity to identify mutants that retain activity (e.g., the ability to bind a CXCL13, e.g., human, murine, or both human and murine CXCL13). Such variants (or derivatives thereof) of “human” or “fully human” antibodies can also be referred to as human or fully human antibodies that are “optimized” or “optimized for antigen binding” and include antibodies that have improved affinity to antigen.

As used herein, the term “chimeric antibody” will be held to mean any antibody wherein the immunoreactive region or site is obtained or derived from a first species and the constant region (which may be intact, partial or modified in accordance with the instant invention) is obtained from a second species. In certain embodiments the target binding region or site will be from a non-human source (e.g., mouse or primate) and the constant region is human (for example, monoclonal antibody (MAb) 1476 described herein).

As used herein, the term “engineered antibody” refers to an antibody in which the variable domain in either the heavy or light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable domain to transfer the antigen binding capacity of one variable domain to another. Rather, it may only be necessary to transfer those residues that are necessary to maintain the activity of the target binding site. In certain embodiments, the humanized antibody comprises 1, 2, or 3 CDRs from a donor variable heavy domain. In another embodiment, the humanized antibody comprises 1, 2, or 3 CDRs from a donor variable light domain.

It is further recognized that the framework regions within the variable domain in a heavy or light chain, or both, of a humanized antibody may comprise solely residues of human origin, in which case these framework regions of the humanized antibody (for example, MAb 5080 or 5261) are referred to as “fully human framework regions.” Alternatively, one or more residues of the framework region(s) of the donor variable domain can be engineered within the corresponding position of the human framework region(s) of a variable domain in a heavy or light chain, or both, of a humanized antibody if necessary to maintain proper binding or to enhance binding to the CXCL13 antigen. A human framework region that has been engineered in this manner would thus comprise a mixture of human and donor framework residues, and is referred to herein as a “partially human framework region.”

For example, humanization of an anti-CXCL13 antibody can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent or mutant rodent CDRs or CDR sequences for the corresponding sequences of a human anti-CXCL13 antibody. See also U.S. Pat. Nos. 5,225,539; 5,585,089; 5,693,761; 5,693,762; and 5,859,205; herein incorporated by reference. The resulting humanized anti-CXCL13 antibody would comprise at least one rodent or mutant rodent CDR within the fully human framework regions of the variable domain of the heavy and/or light chain of the humanized antibody. In some instances, residues within the framework regions of one or more variable domains of the humanized anti-CXCL13 antibody are replaced by corresponding non-human (for example, rodent) residues (see, for example, U.S. Pat. Nos. 5,585,089; 5,693,761; 5,693,762; and 6,180,370), in which case the resulting humanized anti-CXCL13 antibody would comprise partially human framework regions within the variable domain of the heavy and/or light chain.

Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the CDRs correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature 331:522-525 (1986); Riechmann et al., Nature 332:323-329 (1988); and Presta, Curr. Op. Struct. Biol. 2:593-596 (1992); herein incorporated by reference. Accordingly, such “humanized” antibodies may include antibodies wherein substantially less than an intact human variable domain has been substituted by the corresponding sequence from a non-human species. In practice, humanized antibodies are typically human antibodies in which some or all CDR residues and possibly some framework residues are substituted by residues from analogous sites in rodent antibodies. See, for example, U.S. Pat. Nos. 5,225,539; 5,585,089; 5,693,761; 5,693,762; and 5,859,205. See also U.S. Pat. No. 6,180,370, and International Publication No. WO 01/27160, where humanized antibodies and techniques for producing humanized antibodies having improved affinity for a predetermined antigen are disclosed.

As used herein, the terms “linked,” “fused,” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature). Although the reading frame is thus made continuous throughout the fused segments, the segments may be physically or spatially separated by, for example, in-frame linker sequence. For example, polynucleotides encoding the CDRs of an immunoglobulin variable region may be fused, in-frame, but be separated by a polynucleotide encoding at least one immunoglobulin framework region or additional CDR regions, as long as the “fused” CDRs are co-translated as part of a continuous polypeptide.

In certain embodiments the anti-CXCL13 antibodies bind human, primate, murine, or both human and murine CXCL13. In certain embodiments, the anti-CXCL13 antibodies of the invention are humanized.

Suitable biologically active variants of the anti-CXCL13 antibodies of the invention can be used in the methods of the present invention. Such variants will retain the desired binding properties of the parent anti-CXCL13 antibody. Methods for making antibody variants are generally available in the art.

Methods for mutagenesis and nucleotide sequence alterations are well known in the art. See, for example, Walker and Gaastra, eds. (1983) Techniques in Molecular Biology (MacMillan Publishing Company, New York); Kunkel, Proc. Natl. Acad. Sci. USA 82:488-492 (1985); Kunkel et al., Methods Enzymol. 154:367-382 (1987); Sambrook et al. (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.), pp. 345-352, herein incorporated by reference in its entirety. The model of Dayhoff et al. uses the Point Accepted Mutation (PAM) amino acid similarity matrix (PAM 250 matrix) to determine suitable conservative amino acid substitutions. Conservative substitutions, such as exchanging one amino acid with another having similar properties, may be preferred. Examples of conservative amino acid substitutions as taught by the PAM 250 matrix of the Dayhoff et al. model include, but are not limited to, Gly⇄Ala, Val⇄Ile⇄Leu, Asp⇄Glu, Lys⇄Arg, Asn⇄Gln, and Phe⇄Trp⇄Tyr.

In constructing variants of the anti-CXCL13 binding molecule, e.g., an antibody or antigen-binding fragment thereof, polypeptides of interest, modifications are made such that variants continue to possess the desired properties, e.g., being capable of specifically binding to a CXCL13, e.g., human, primate, murine, or both human and murine CXCL13. Obviously, any mutations made in the DNA encoding the variant polypeptide must not place the sequence out of reading frame and preferably will not create complementary regions that could produce secondary mRNA structure. See, e.g., EP Pat. No. EP0075444 B1.

Methods for measuring anti-CXCL13 binding molecule, e.g., an antibody or antigen-binding fragment thereof, binding specificity include, but are not limited to, standard competitive binding assays, assays for monitoring immunoglobulin secretion by T cells or B cells, T cell proliferation assays, apoptosis assays, ELISA assays, and the like. See, for example, such assays disclosed in WO 93/14125; Shi et al., Immunity 13:633-642 (2000); Kumanogoh et al., J Immunol 169:1175-1181 (2002); Watanabe et al., J Immunol 167:4321-4328 (2001); Wang et al., Blood 97:3498-3504 (2001); and Giraudon et al., J Immunol 172(2):1246-1255 (2004), all of which are herein incorporated by reference.

When discussed herein whether any particular polypeptide, including the constant regions, CDRs, VH domains, or VL domains disclosed herein, is at least about 65%, about 70%, about 75%, about 80%, about 85%, about 90%, about 91%, about 92%, about 93%, about 94%, about 95%, about 96%, about 97%, about 98%, about 99%, or even about 100% identical to another polypeptide, the % identity can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman (1981) Adv. Appl. Math. 2:482-489, to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

For purposes of the present invention, percent sequence identity may be determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant may, for example, differ from a reference anti-CXCL13 antibody (e.g., MAb 5261, MAb 5378, MAb 5080, MAb 1476, 3D2, or 3C9) by as few as 1 to 15 amino acid residues, as few as 1 to 10 amino acid residues, such as 6-10, as few as 5, as few as 4, 3, 2, or even 1 amino acid residue.

The precise chemical structure of a polypeptide capable of specifically binding CXCL13 and retaining the desired CXCL13 blocking activity depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of anti-CXCL13 antibodies as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of an anti-CXCL13 antibody used herein so long as the desired properties of the anti-CXCL13 antibody are not destroyed. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity. Such alterations that do not destroy the desired properties (e.g., binding specificity for CXCL13, binding affinity, and/or CXCL13 blocking activity) do not remove the polypeptide sequence from the definition of anti-CXCL13 antibodies of interest as used herein.

The art provides substantial guidance regarding the preparation and use of polypeptide variants. In preparing the anti-CXCL13 binding molecule, e.g., an antibody or antigen-binding fragment thereof, variants, one of skill in the art can readily determine which modifications to the native proteins nucleotide or amino acid sequence will result in a variant that is suitable for use as a therapeutically active component of a pharmaceutical composition used in the methods of the present invention.

The constant region of an anti-CXCL13 antibody may be mutated to alter effector function in a number of ways. For example, see U.S. Pat. No. 6,737,056B1 and U.S. Patent Application Publication No. 2004/0132101A1, which disclose Fc mutations that optimize antibody binding to Fc receptors.

In certain anti-CXCL13 antibodies, the Fc portion may be mutated to decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well known immunological techniques without undue experimentation.

Anti-CXCL13 antibodies of the invention also include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the derivative may contain one or more non-classical amino acids.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., binding specificity for CXCL13, binding affinity, and/or CXCL13 blocking activity).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybridoma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one epitope of a CXCL13 polypeptide) can be determined using techniques described herein or by routinely modifying techniques known in the art.

In some embodiments, the anti-CXCL13 antibodies or antigen-binding fragments thereof of the invention inhibit CXCL13 activity. “Anti-CXCL13 activity” or “CXCL13 blocking activity” can include activity which modulates one or more of the following activities associated with CXCL13: blockade of CXCL13 interaction with its receptor resulting in interference with B cell and follicular B-helper T cell migration into inflamed tissues and germinal center formation (e.g., in the case of autoimmune diseases); inhibition of cancer cell proliferation and ability to spread in oncological disorders; or any other activity association with CXCL13-expressing cells. Anti-CXCL13 activity can also be attributed to a decrease in incidence or severity of diseases associated with CXCL13 expression, including, but not limited to, certain types of autoimmune diseases (e.g., Multiple sclerosis, arthritis (e.g., Rheumatoid arthritis), chronic gastritis, gastric lymphomas, transplant rejection, Sjogren syndrome (SS), Systemic Lupus Erythematosis (SLE), active mixed cryoglobulinemia (MC) vasculitis in Hepatitis C virus infection, Juvenile dermatomyositis, and Myastenia Gravis) and certain cancers (e.g., Burkitt's lymphoma, Non-Hodgkin Lymphoma, MALT lymphoma (e.g., gastric MALT lymphoma), Carcinoma (e.g., colon, prostate, breast, stomach, esophageal, and pancreatic), and Chronic lymphocytic leukemia (CLL)) as well as other inflammatory diseases such as Helicobacter infection induced inflammatory diseases, e.g., gastritis, ulcers, and gastric mucosal lesions.

In some of these embodiments, the anti-CXCL13 antibody or antigen-binding fragment thereof inhibits CXCL13 from binding to a CXCL13 receptor. In some of these embodiments, the CXCL13 receptor is CXCR5. In other embodiments, the CXCL13 receptor is CXCR3.

As used herein, the terms “CXCR5” and “CXCR5 polypeptide” are used interchangeably. In certain embodiments, CXCR5 may include a full-sized CXCR5 or a fragment thereof, or a CXCR5 variant polypeptide, wherein the fragment of CXCR5 or CXCR5 variant polypeptide retains some or all functional properties of the full-sized CXCR5. The terms “CXCR5” and “CXCR5 polypeptide” also encompass a soluble form of CXCR5. As used herein, the term “soluble form of CXCR5” is a form of CXCR5 that is not bound to a plasma membrane. Full-length CXCR5 is a seven transmembrane receptor. Therefore, non-limiting examples of a soluble form of CXCR5 include fragments of CXCR5 that consist essentially of the extracellular domain (e.g., about the first 60 amino acids). The human CXCR5 polynucleotide and polypeptide sequences are known in the art and provided herein as SEQ ID NOs: 27 and 28, respectively. The murine CXCR5 polynucleotide and polypeptide sequences are known in the art and provided herein as SEQ ID NOs: 29 and 30, respectively.

As used herein, the terms “CXCR3” and “CXCR3 polypeptide” are used interchangeably. In certain embodiments, CXCR3 may include a full-sized CXCR3 or a fragment thereof, or a CXCR3 variant polypeptide, wherein the fragment of CXCR3 or CXCR3 variant polypeptide retains some or all functional properties of the full-sized CXCR3. The terms “CXCR3” and “CXCR3 polypeptide” also encompass a soluble form of CXCR3. As used herein, the term “soluble form of CXCR3” is a form of CXCR3 that is not bound to a plasma membrane. Full-length CXCR3 is a seven transmembrane receptor. Therefore, non-limiting examples of a soluble form of CXCR3 include fragments of CXCR3 that consist essentially of the extracellular domain. Human CXCR3 sequences are known in the art and provided herein as SEQ ID NOs: 31 and 32 are human CXCR3 variant 1 and variant 3, respectively.

In certain embodiments, the anti-CXCL13 antibodies of the invention comprise at least one optimized complementarity-determining region (CDR). By “optimized CDR” is intended that the CDR has been modified and optimized sequences selected based on the sustained or improved binding affinity and/or anti-CXCL13 activity that is imparted to an anti-CXCL13 antibody comprising the optimized CDR.

As discussed in more detail elsewhere herein, anti-CXCL13 binding molecules, e.g., antibodies of the invention, or antigen-binding fragments, variants, or derivatives thereof, may further be recombinantly fused to a heterologous polypeptide at the N- or C-terminus or chemically conjugated (including covalent and non-covalent conjugations) to polypeptides or other compositions. For example, anti-CXCL13 antibodies may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

Anti-CXCL13 antibodies of the invention, or antigen-binding fragments, variants, or derivatives thereof, may include derivatives that are modified, i.e., by the covalent attachment of any type of molecule to the antibody such that covalent attachment does not prevent the antibody binding anti-CXCL13. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the derivative may contain one or more non-classical amino acids.

Anti-CXCL13 binding molecules, e.g., antibodies of the invention, or antigen-binding fragments, variants, or derivatives thereof, can be composed of amino acids joined to each other by peptide bonds or modified peptide bonds, i.e., peptide isosteres, and may contain amino acids other than the 20 gene-encoded amino acids. For example, anti-CXCL13 antibodies may be modified by natural processes, such as posttranslational processing, or by chemical modification techniques that are well known in the art. Such modifications are well described in basic texts and in more detailed monographs, as well as in a voluminous research literature. Modifications can occur anywhere in the anti-CXCL13 binding molecule, including the peptide backbone, the amino acid side-chains and the amino or carboxyl termini, or on moieties such as carbohydrates. It will be appreciated that the same type of modification may be present in the same or varying degrees at several sites in a given anti-CXCL13 binding molecule. Also, a given anti-CXCL13 binding molecule may contain many types of modifications. Anti-CXCL13 binding molecules may be branched, for example, as a result of ubiquitination, and they may be cyclic, with or without branching. Cyclic, branched, and branched cyclic anti-CXCL13 binding molecule may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure and Molecular Properties, T. E. Creighton, W. H. Freeman and Company, NY; 2nd ed. (1993); Johnson, ed. (1983) Posttranslational Covalent Modification of Proteins (Academic Press, NY), pgs. 1-12; Seifter et al., Meth. Enzymol. 182:626-646 (1990); Rattan et al., Ann. NY Acad. Sci. 663:48-62 (1992)).

The present invention also provides for fusion proteins comprising an anti-CXCL13 antibody, or antigen-binding fragment, variant, or derivative thereof, and a heterologous polypeptide. The heterologous polypeptide to which the antibody is fused may be useful for function or is useful to target the anti-CXCL13 polypeptide expressing cells.

In one embodiment, a fusion protein of the invention comprises, consists essentially of, or consists of, a polypeptide having the amino acid sequence of any one or more of the VH domains of an antibody of the invention or the amino acid sequence of any one or more of the VL domains of an antibody of the invention or fragments or variants thereof, and a heterologous polypeptide sequence.

In another embodiment, a fusion protein for use in the diagnostic and treatment methods disclosed herein comprises, consists essentially of, or consists of a polypeptide having the amino acid sequence of any one, two, three of the CDRs of the VH domain of an anti-CXCL13 antibody, or fragments, variants, or derivatives thereof, and/or the amino acid sequence of any one, two, three of the CDRs of the VL domain an anti-CXCL13 antibody, or fragments, variants, or derivatives thereof, and a heterologous polypeptide sequence. In one embodiment, a fusion protein comprises a polypeptide having the amino acid sequence of at least one VH domain of an anti-CXCL13 antibody of the invention and the amino acid sequence of at least one VL domain of an anti-CXCL13 antibody of the invention or fragments, derivatives or variants thereof, and a heterologous polypeptide sequence. Preferably, the VH and VL domains of the fusion protein correspond to a single source antibody (or scFv or Fab fragment) that specifically binds at least one epitope of CXCL13. In yet another embodiment, a fusion protein for use in the diagnostic and treatment methods disclosed herein comprises a polypeptide having the amino acid sequence of any one, two, three or more of the CDRs of the VH domain of an anti-CXCL13 antibody and the amino acid sequence of any one, two, three or more of the CDRs of the VL domain of an anti-CXCL13 antibody, or fragments or variants thereof, and a heterologous polypeptide sequence. Preferably, two, three, four, five, six, or more of the CDR(s) of the VH domain or VL domain correspond to single source antibody (or scFv or Fab fragment) of the invention. Nucleic acid molecules encoding these fusion proteins are also encompassed by the invention.

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670(1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

As discussed elsewhere herein, anti-CXCL13 binding molecules, e.g., antibodies of the invention, or antigen-binding fragments, variants, or derivatives thereof, may be fused to heterologous polypeptides to increase the in vivo half life of the polypeptides or for use in immunoassays using methods known in the art. For example, in one embodiment, PEG can be conjugated to the anti-CXCL13 antibodies of the invention to increase their half-life in vivo. See Leong et al., Cytokine 16:106 (2001); Adv. in Drug Deliv. Rev. 54:531 (2002); or Weir et al., Biochem. Soc. Transactions 30:512 (2002).

Moreover, anti-CXCL13 binding molecules, e.g., antibodies of the invention, or antigen-binding fragments, variants, or derivatives thereof, can be fused to marker sequences, such as a peptide to facilitate their purification or detection. In certain embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Fusion proteins can be prepared using methods that are well known in the art (see for example U.S. Pat. Nos. 5,116,964 and 5,225,538). The precise site at which the fusion is made may be selected empirically to optimize the secretion or binding characteristics of the fusion protein. DNA encoding the fusion protein is then transfected into a host cell for expression.

Anti-CXCL13 binding molecules, e.g., antibodies of the present invention, or antigen-binding fragments, variants, or derivatives thereof, may be used in non-conjugated form or may be conjugated to at least one of a variety of molecules, e.g., to improve the therapeutic properties of the molecule, to facilitate target detection, or for imaging or therapy of the patient. Anti-CXCL13 binding molecules, e.g., antibodies of the invention, or antigen-binding fragments, variants, or derivatives thereof, can be labeled or conjugated either before or after purification, or when purification is performed.

In particular, anti-CXCL13 antibodies of the invention, or antigen-binding fragments, variants, or derivatives thereof, may be conjugated to therapeutic agents, prodrugs, peptides, proteins, enzymes, viruses, lipids, biological response modifiers, pharmaceutical agents, or PEG.

Those skilled in the art will appreciate that conjugates may also be assembled using a variety of techniques depending on the selected agent to be conjugated. For example, conjugates with biotin are prepared, e.g., by reacting a binding polypeptide with an activated ester of biotin such as the biotin N-hydroxysuccinimide ester. Similarly, conjugates with a fluorescent marker may be prepared in the presence of a coupling agent, e.g., those listed herein, or by reaction with an isothiocyanate, preferably fluorescein-isothiocyanate. Conjugates of the anti-CXCL13 antibodies of the invention, or antigen-binding fragments, variants, or derivatives thereof, are prepared in an analogous manner.

The present invention further encompasses anti-CXCL13 binding molecules, e.g., antibodies of the invention, or antigen-binding fragments, variants, or derivatives thereof, conjugated to a diagnostic or therapeutic agent. The anti-CXCL13 antibodies, including antigen-binding fragments, variants, and derivatives thereof, can be used diagnostically to, for example, monitor the development or progression of a disease as part of a clinical testing procedure to, e.g., determine the efficacy of a given treatment and/or prevention regimen. For example, detection can be facilitated by coupling the anti-CXCL13 antibody, or antigen-binding fragment, variant, or derivative thereof, to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In, ⁹⁰Y, or ⁹⁹Tc.

An anti-CXCL13 binding molecule, e.g., an antibody, or antigen-binding fragment, variant, or derivative thereof, may be conjugated to a therapeutic moiety such as a cytotoxin, a therapeutic agent or a radioactive metal ion. A cytotoxin or cytotoxic agent includes any agent that is detrimental to cells.

An anti-CXCL13 binding molecule, e.g., an antibody, or antigen-binding fragment, variant, or derivative thereof, also can be detectably labeled by coupling it to a chemiluminescent compound. The presence of the chemiluminescent-tagged anti-CXCL13 binding molecule is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which an anti-CXCL13 antibody, or antigen-binding fragment, variant, or derivative thereof, can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md.; Diagnostic Horizons 2:1-7 (1978); Voller et al., J. Clin. Pathol. 31:507-520 (1978); Butler, Meth. Enzymol. 73:482-523 (1981); Maggio, ed. (1980) Enzyme Immunoassay, CRC Press, Boca Raton, Fla.; Ishikawa et al., eds. (1981) Enzyme Immunoassay (Kgaku Shoin, Tokyo). The enzyme, which is bound to the anti-CXCL13 antibody will react with an appropriate substrate, e.g., a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the anti-CXCL13 binding molecule, e.g., antibody, or antigen-binding fragment, variant, or derivative thereof, it is possible to detect the binding molecule through the use of a radioimmunoassay (RIA) (see, for example, Weintraub (March, 1986) Principles of Radioimmunoassays, Seventh Training Course on Radioligand Assay Techniques (The Endocrine Society), which is incorporated by reference herein). The radioactive isotope can be detected by means including, but not limited to, a gamma counter, a scintillation counter, or autoradiography.

An anti-CXCL13 binding molecule, e.g., antibody, or antigen-binding fragment, variant, or derivative thereof, can also be detectably labeled using fluorescence emitting metals such as 152Eu, or others of the lanthanide series. These metals can be attached to the binding molecule using such metal chelating groups as diethylenetriaminepentacetic acid (DTPA) or ethylenediaminetetraacetic acid (EDTA).

Techniques for conjugating various moieties to an antibody, e.g., an anti-CXCL13 antibody or antigen-binding fragment, variant, or derivative thereof, are well known, see, e.g., Amon et al. (1985) “Monoclonal Antibodies for Immunotargeting of Drugs in Cancer Therapy,” in Monoclonal Antibodies and Cancer Therapy, ed. Reisfeld et al. (Alan R Liss, Inc.), pp. 243-56; Hellstrom et al. (1987) “Antibodies for Drug Delivery,” in Controlled Drug Delivery, ed. Robinson et al. (2nd ed.; Marcel Dekker, Inc.), pp. 623-53); Thorpe (1985) “Antibody Carriers of Cytotoxic Agents in Cancer Therapy: A Review,” in Monoclonal Antibodies '84: Biological and Clinical Applications, ed. Pinchera et al., pp. 475-506; “Analysis, Results, and Future Prospective of the Therapeutic Use of Radiolabeled Antibody in Cancer Therapy,” in Monoclonal Antibodies for Cancer Detection and Therapy, ed. Baldwin et al., Academic Press, pp. 303-16 (1985); and Thorpe et al., Immunol. Rev. 62:119-58 (1982).

Isolated polypeptides consisting of epitopes for binding an anti-CXCL13 antibody are provided.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide useful in the presently disclosed methods may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides that do not possess a defined three-dimensional structure, but rather can adopt a large number of different conformations, are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu No particular level of purification is required. For example, an isolated polypeptide can be removed from its native or natural environment. Recombinantly produced polypeptides and proteins expressed in host cells are considered isolated for purpose of the invention, as are native or recombinant polypeptides that have been separated, fractionated, or partially or substantially purified by any suitable technique.

Also included as polypeptides of the invention are fragments, derivatives, analogs, or variants of polypeptides, and any combination thereof. The terms “fragment,” “variant,” “derivative,” and “analog” when referring to anti-CXCL13 antibodies or antibody or epitope polypeptides include any polypeptides that retain at least some of the antigen-binding properties of the corresponding antibody or antibody polypeptide or at least some of the immunogenic properties of a CXCL13 epitope sequence. Fragments of polypeptides include proteolytic fragments, as well as deletion fragments, in addition to specific antibody fragments discussed elsewhere herein. Variants of anti-CXCL13 antibodies and CXCL13 epitope polypeptides include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions, or additions. Variant polypeptides may also be referred to herein as “polypeptide analogs.” As used herein a “derivative” of an anti-CXCL13 antibody, antibody polypeptide, or epitope polypeptide refers to a subject polypeptide having one or more residues chemically derivatized by reaction of a functional side group. Also included as “derivatives” are those peptides that contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine. Derivatives of anti-CXCL13 antibodies and antibody polypeptides, may include polypeptides that have been altered so as to exhibit additional features not found on the reference antibody or antibody polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

In some embodiments, the epitope polypeptides of the invention comprise the sequence set forth in SEQ ID NO: 26 or a variant or fragment thereof. In some of these embodiments, the epitope polypeptide comprises at least 5, at least 6, at least 7, at least 8, at least 9, at least 10, at least 11, at least 12, at least 13, or at least 14 contiguous amino acid residues of SEQ ID NO: 26. In other embodiments, the epitope polypeptide has a sequence having at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or greater identity to SEQ ID NO: 26. In still other embodiments, the epitope polypeptide has a sequence identical to SEQ ID NO: 26, except for 10, 9, 8, 7, 6, 5, 4, 3, 2, or 1 amino acid substitutions, which can be conservative amino acid substitutions. In certain embodiments, the epitope polypeptide has a sequence identical to SEQ ID NO: 26, except for 4 or fewer amino acid substitutions.

Isolated polynucleotides that encode epitope polypeptides are also provided. The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, that has been removed from its native environment. For example, a recombinant polynucleotide encoding an anti-CXCL13 binding molecule, e.g., an antibody or antigen binding fragment thereof, contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

As used herein, a “coding region” is a portion of a nucleic acid that consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions useful in the presently disclosed methods can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding an anti-CXCL13 antibody or fragment, variant, or derivative thereof. Heterologous coding regions include without limitation specialized elements or motifs, such as a secretory signal peptide or a heterologous functional domain.

In certain embodiments, the polynucleotide or nucleic acid of the invention is DNA. In the case of DNA, a polynucleotide comprising a nucleic acid that encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to, ribosome binding sites, translation initiation and termination codons, and elements derived from picornaviruses (particularly an internal ribosome entry site, or IRES, also referred to as a CITE sequence).

In other embodiments, a polynucleotide of the invention is RNA, for example, in the form of messenger RNA (mRNA).

Polynucleotide and nucleic acid coding regions of the invention may be associated with additional coding regions that encode secretory or signal peptides, which direct the secretion of a polypeptide encoded by a polynucleotide of the present invention. According to the signal hypothesis, proteins secreted by mammalian cells have a signal peptide or secretory leader sequence that is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

The CXCL13 epitope polypeptides and polynucleotides encoding the same can be used to produce anti-CXCL13 antibodies. Any method known in the art for producing polyclonal or monoclonal antibodies may be used to generate anti-CXCL13 antibodies using the presently disclosed CXCL13 epitope polypeptides and polynucleotides encoding the same, including those methods described elsewhere herein (see, for example, Example 1). In some embodiments, an animal is immunized with a presently disclosed CXCL13 epitope polypeptide. In other embodiments, anti-CXCL13 monoclonal antibodies are produced using the hybridoma method described by Kohler et al. (1975) Nature 256:495, which is herein incorporated by reference, or may be made by recombinant DNA methods. In some of these embodiments, an animal is immunized with a presently disclosed CXCL13 epitope polypeptide, antibody-producing cells (e.g., spleen cells) are isolated from the animal and fused to immortalized cells in culture to produce hybridoma cells, and the monoclonal antibodies can be isolated from the hybridoma cells.

Lymphoid chemokine CXCL13 is expressed by follicular dendritic cells (FDCs) and macrophages. Through its receptor, CXCR5, which is found on a variety of immune cells (e.g., B cells, follicular helper T cells, and recently-activated T cells), CXCL13 induces intracellular changes necessary for maintenance of immune system homeostasis, lymphoid organogenesis, leukocyte trafficking and chemotactic migration as well as development of secondary lymphoid tissue (e.g. germinal centers). Overexpression of CXCL13 and its receptor CXCR5 have been implicated into a variety of autoimmune diseases (e.g., Multiple sclerosis (see, e.g., Corcione et al., PNAS 101(30):11064-11069 (2004); Serafini et al., Brain Pathol. 14:164-174 (2004); Magliozzi et al., Brain 130: 1089-1104 (2007)), arthritis (e.g., Rheumatoid arthritis (see, e.g., Rioja et al., Arthritis & Rheumatism 58(8)2257-2267 (2008): Shi et al., J. Immuno. 166:650-655 (2001); Schmutz et al., Arthritis Restearch and Therapy 7:R217-R229 (2005); Hjelmström et al., J. Leukocyte Bio. 69:331-339 (2001)), chronic gastritis (see, e.g., Hjelmström et al.; Mazzucchelli et al., Brain 130:1089-1104 (2007)), gastric lymphomas (see, e.g., id.; Nobutani et al., FEMS Immunol Med Microbiol 60:156-164 (2010)), transplant rejection (see, e.g., Steinmetz et al., Kidney International 67:1616-1621 (2005)), Sjogren syndrome (SS) (see, e.g., Barone et al., J. Immuno. 180:5130-5140 (2008); Hjelmström et al.), Systemic Lupus Erythematosis (SLE) (see, e.g., Steinmetz et al., Lee et al., J. Rheum. 37(1):45-52 (2010); Schiffer et al., J. Immun. 171:489-497 (2003)), active mixed cryoglobulinemia (MC) vasculitis in Hepatitis C virus infection (see, e.g., Sansonno et al., Blood 112(5):1620-1627 (2008)), Juvenile dermatomyositis (see, e.g., de Padilla et al., Arthritis & Rheumatism 60(4):1160-1172 (2009)), and Myastenia Gravis (see, e.g., Matsumoto et al., J. Immuno. 176:5100-5107 (2006); Meraouna et al., Blood 108(2):432-440 (2006); Saito et al., J. Neuroimmunol 170:172-178 (2005)) and certain cancers (e.g., Burkitt's lymphoma (see, e.g., Förster et al., Blood 84:830-840 (1994); Förster et al., Cell 87:1037-1047 (1996)), Non-Hodgkin Lymphoma (see, e.g., Trentin et al., Ann. Rev. Immunol. 6:251-81 (1988); Gong et al., J. Immunol. 174: 817-826 (2005); Hamaguchi et al., J. Immunol. 174:4389-4399 (2005)), Carcinoma (e.g., colon and pancreatic) (see, e.g., Günther et al., Int. J. Cancer 116:726-733 (2005); Meijer et al., Cancer Res. 66: 9576-9582 (2006)), breast cancer (see, e.g., Panse et al., British Journal of Cancer 99:930-938 (2008)), Chronic lymphocytic leukemia (CLL) (see, e.g., Bürkle et al., Blood 110:3316-3325 (2007)), and prostate cancer (see, e.g., Singh et al., Cancer Letters 283 (1):29-35 (2009)). Methods of the invention for inhibition of CXCL13 activity would be expected to have a therapeutic effect on the above mentioned disorders.

Certain methods of the invention are directed to the use of anti-CXCL13 binding molecules, e.g., antibodies, including antigen-binding fragments, variants, and derivatives thereof, to treat patients having a disease associated with CXCL13-expressing cells, e.g., CXCL13-overexpressing cells. By “CXCL13-expressing cell” is intended normal and malignant cells expressing CXCL13 antigen. Methods for detecting CXCL13 expression in cells are well known in the art and include, but are not limited to, PCR techniques, immunohistochemistry, flow cytometry, Western blot, ELISA, and the like.

Although the following discussion refers to diagnostic methods and treatment of various diseases and disorders with an anti-CXCL13 antibody of the invention, the methods described herein are also applicable to the antigen-binding fragments, variants, and derivatives of these anti-CXCL13 antibodies that retain the desired properties of the anti-CXCL13 antibodies of the invention, e.g., capable of specifically binding CXCL13, e.g., human, primate, mouse, or human and mouse CXCL13, and having CXCL13 neutralizing activity.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis, lupus, arthritis, or cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cows, and so on.

As used herein, phrases such as “a subject that would benefit from administration of an anti-CXCL13 antibody” and “an animal in need of treatment” includes subjects, such as mammalian subjects, that would benefit from administration of an anti-CXCL13 antibody used, e.g., for detection of an anti-CXCL13 polypeptide (e.g., for a diagnostic procedure) and/or from treatment, i.e., palliation or prevention of a disease, with an anti-CXCL13 antibody. As described in more detail herein, an anti-CXCL13 antibody can be used in unconjugated form or can be conjugated, e.g., to a drug, prodrug, or an isotope.

In one embodiment, treatment includes the application or administration of an anti-CXCL13 binding molecule, e.g., an antibody or antigen binding fragment thereof, of the current invention to a patient, or application or administration of the anti-CXCL13 binding molecule to an isolated tissue or cell line from a patient, where the patient has a disease, a symptom of a disease, or a predisposition toward a disease. In another embodiment, treatment is also intended to include the application or administration of a pharmaceutical composition comprising the anti-CXCL13 binding molecule, e.g., an antibody or antigen binding fragment thereof, of the current invention to a patient, or application or administration of a pharmaceutical composition comprising the anti-CXCL13 binding molecule to an isolated tissue or cell line from a patient, who has a disease, a symptom of a disease, or a predisposition toward a disease.

The anti-CXCL13 binding molecules, e.g., antibodies or binding fragments thereof, of the present invention are useful for the treatment of various malignant and non-malignant tumors. By “anti-tumor activity” is intended a reduction in the rate of malignant CXCL13-expressing cell proliferation or accumulation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. For example, therapy with at least one anti-CXCL13 antibody causes a physiological response that is beneficial with respect to treatment of disease states associated with CXCL13-expressing cells in a human.

In one embodiment, the invention relates to anti-CXCL13 binding molecules, e.g., antibodies or binding fragments thereof, according to the present invention for use as a medicament, in particular for use in the treatment or prophylaxis of cancer or for use in a precancerous condition or lesion. In certain embodiments, an anti-CXCL13 binding molecule, e.g., an antibody or binding fragment thereof, e.g., MAb 5261, of the invention is used for the treatment of a CXCL13 over-expressing cancer. In certain embodiments, an anti-CXCL13 binding molecule, e.g., an antibody or binding fragment thereof, of the invention is used for the treatment of a CXCL13 expressing or over-expressing leukemia, lymphoma (e.g., MALT lymphoma), colon, pancreatic, stomach, esophageal, breast, or prostate cancer. In one embodiment, an anti-CXCL13 binding molecule, e.g., an antibody or binding fragment thereof, of the invention is used for the treatment of a carcinoma, e.g., colon or prostate carcinoma. In one embodiment, the an anti-CXCL13 binding molecule, e.g., an antibody or binding fragment thereof, of the invention is used for the treatment of a CXCR5 expressing or over-expressing cancer.

The effectiveness of an anti-CXCL13 binding molecule, e.g., an antibody or binding fragment thereof, for the treatment or prevention of cancer can be shown using animal models. For example, the effectiveness of an anti-CXCL13 binding molecule, e.g., an antibody or binding fragment thereof, of the invention for the treatment or prevention of prostate cancer can be shown using an animal model for prostate cancer, e.g., mice injected with PC3-luc human prostate carcinoma cells and treated with an anti-CXCL13 binding molecule of the invention. In another example, the effectiveness of an anti-CXCL13 binding molecule, e.g., an antibody or binding fragment thereof, of the invention for the treatment or prevention of colon cancer can be shown using an animal model for colon cancer, e.g., mice injected with CT26 colon carcinoma cells and treated with an anti-CXCL13 binding molecule of the invention. In another example, the effectiveness of an anti-CXCL13 binding molecule, e.g., an antibody or binding fragment thereof, of the invention for the treatment or prevention of MALT lymphoma can be shown using an animal model for gastric MALT lymphoma, e.g., mice infected with Helicobacter bacteria (see Nobutani et al. (2010)) and treated with an anti-CXCL13 binding molecule of the invention. The Nobutani et al. model may also be used to assess the effectiveness of an anti-CXCL13 binding molecule, e.g., an antibody or binding fragment thereof, of the invention for the reduction of gastric lymphoid follicles of Helicobacter-infected tissues.

In accordance with the methods of the present invention, at least one anti-CXCL13 binding molecule, e.g., an antibody or antigen binding fragment thereof, as defined elsewhere herein is used to promote a positive therapeutic response with respect to a malignant human cell. By “positive therapeutic response” with respect to cancer treatment is intended an improvement in the disease in association with the anti-tumor activity of these binding molecules, e.g., antibodies or fragments thereof, and/or an improvement in the symptoms associated with the disease. That is, an anti-proliferative effect, the prevention of further tumor outgrowths, a reduction in tumor size, a decrease in tumor vasculature, a reduction in the number of cancer cells, and/or a decrease in one or more symptoms associated with the disease can be observed. Thus, for example, an improvement in the disease may be characterized as a complete response. By “complete response” is intended an absence of clinically detectable disease with normalization of any previously abnormal radiographic studies, bone marrow, and cerebrospinal fluid (CSF). Such a response must persist for at least one month following treatment according to the methods of the invention. Alternatively, an improvement in the disease may be categorized as being a partial response. By “partial response” is intended at least about a 50% decrease in all measurable tumor burden (i.e., the number of tumor cells present in the subject) in the absence of new lesions and persisting for at least one month. Such a response is applicable to measurable tumors only.

Tumor response can be assessed for changes in tumor morphology (i.e., overall tumor burden, tumor cell count, and the like) using screening techniques such as bioluminescent imaging, for example, luciferase imaging, bone scan imaging, and tumor biopsy sampling including bone marrow aspiration (BMA). In addition to these positive therapeutic responses, the subject undergoing therapy with the anti-CXCL13 binding molecule, e.g., an antibody or antigen-binding fragment thereof, may experience the beneficial effect of an improvement in the symptoms associated with the disease. For example, the subject may experience a decrease in the so-called B symptoms, e.g., night sweats, fever, weight loss, and/or urticaria.

The anti-CXCL13 binding molecules, e.g., antibodies or antigen binding fragments thereof, described herein may also find use in increasing immunoglobulin A (IgA) levels in subjects having a deficiency thereof or for treating an inflammatory or autoimmune disorder in subjects having an IgA deficiency, as described in U.S. Provisional Application No. 61/759,108, filed Jan. 31, 2013, entitled “Methods for Increasing Immunoglobulin A Levels,” which is herein incorporated by reference in its entirety.

The anti-CXCL13 binding molecules, e.g., antibodies or antigen binding fragments thereof, described herein may also find use in the treatment or prevention of inflammatory diseases and deficiencies or disorders of the immune system that are associated with CXCL13 expressing cells Inflammatory diseases are characterized by inflammation and tissue destruction, or a combination thereof. By “anti-inflammatory activity” is intended a reduction or prevention of inflammation. “Inflammatory disease” includes any inflammatory immune-mediated process where the initiating event or target of the immune response involves non-self antigen(s), including, for example, alloantigens, xenoantigens, viral antigens, bacterial antigens, unknown antigens, or allergens.

In one embodiment, the inflammatory disease is associated with a bacterial infection, e.g., a Helicobacter infection, e.g., a H. pylori, H. heilmannii, H. acinonychis, H. anseris, H. aurati, H. baculiformis, H. bilis, H. bizzozeronii, H. brantae, H. candadensis, H. canis, H. cholecystus, H. cinaedi, H. cynogastricus, H. equorum, H. felis, H. fenelliae, H. ganmani, H. hepaticus, H. mesocricetorum, H. marmotae, H. muridarum, H. mustelae, H. pametensis, H. pullorum, H. rappini, H. rodentium, H. salomonis, H. suis, H. trogontum, H. typhlonius, and H. winghamensis infection. In a certain embodiments, the Helicobacter infection is a H. pylori or a H. heilmannii infection. In a further embodiment, the Helicobacter-associated inflammatory disease is MALT lymphoma, a gastric cancer (e.g., esophageal or stomach cancer), a gastric or duodenal ulcer, gastritis (an inflammation of the stomach lining), or a gastric lesion (see, e.g., Chen et al., J Clin Pathol 55(2):133-7 (2002); Genta et al., Hum Pathol 24(6):577-83 (1993); Okiyama et al., Pathol Int 55(7):398-404 (2005)).

In one embodiment, the inflammatory disease is an inflammatory disorder of the peripheral or central nervous system.

Further, for purposes of the present invention, the term “inflammatory disease(s)” includes, but is not limited to, “autoimmune disease(s).” As used herein, the term “autoimmunity” is generally understood to encompass inflammatory immune-mediated processes involving “self” antigens. In autoimmune diseases, self antigen(s) trigger host immune responses.

In one embodiment, the anti-CXCL13 binding molecule, e.g., an antibody or antigen binding fragment, of the invention is used to treat or prevent multiple sclerosis (MS). MS, also known as disseminated sclerosis or encephalomyelitis disseminata, is an autoimmune condition in which the immune system attacks the central nervous system, leading to demyelination. The name “multiple sclerosis” refers to the scars (scleroses, also referred to as plaques or lesions) that form in the nervous system. MS lesions commonly involve white matter areas close to the ventricles of the cerebellum, brain stem, basal ganglia and spinal cord, and the optic nerve. MS results in destruction of oligodendrocytes, the cells responsible for creating and maintaining the myelin sheath. MS results in a thinning or complete loss of myelin and, as the disease advances, transection of axons.

Neurological symptoms can vary with MS, and the disease often progresses to physical and cognitive disability. MS takes several forms, with new symptoms occurring either in discrete attacks (relapsing forms) or slowly accumulating over time (progressive forms). Between attacks, symptoms may go away completely, but permanent neurological damage often results, especially as the disease advances.

Experimental Autoimmune Encephalomyelitis (EAE) is a widely accepted animal model of multiple sclerosis. EAE is a demyelinating disease of the CNS that progressively results in escalating degrees of ascending paralysis with inflammation primarily targeting the spinal cord. The disease can assume an acute, chronic or relapsing-remitting course that is dependent upon the method of induction and type of animal used. Bagaeva et al. has shown that follicles containing B-cells and CXCL13-expressing dendritic cells formed in inflamed meninges of mice with relapsing-remitting EAE with levels of CXCL13 expression rising steadily throughout the course of disease. CXCL13-deficient mice experienced mild disease with decreased relapse rate, and blockade of CXCL13 with anti-CXCL13 MAb led to the disease attenuation in passively induced EAE in B10.PL mice. Bagaeva et al., J. Immuno. 176:7676-7685 (2006).

Neutralization of CXCL13 using an anti-CXCL13 monoclonal antibody or antigen binding fragment thereof of the invention, e.g., MAb 5261, may be used to reduce the severity of MS through several different mechanisms, e.g., blockade of CXCL13 interaction with its receptor resulting, e.g., in interference with B and follicular B-helper T cell migration into inflamed tissues and germinal center formation.

In one embodiment, the anti-CXCL13 binding molecule, e.g., an antibody or antigen binding fragment, of the invention is used to treat or prevent systemic lupus erythematosus (SLE or lupus). SLE is an autoimmune disease that involves multiple organs and is characterized by the spontaneous formation of ectopic germinal centers and autoantibody production against a number of nuclear antigens. SLE most often affects the heart, joints, skin, lungs, blood vessels, liver, kidneys, and nervous system.

Activated T cells, B cells and their migration-promoting chemokine CXCL13 play critical roles in the formation of organized lymphoid tissue seen in inflamed ectopic sites of multiple autoimmune disorders including SLE. Lupus-prone New Zealand Black X New Zealand White F1 (NZB/NZWF1) mice spontaneously develop high-titer anti-dsDNA antibodies and severe proliferative glomerulonephritis caused by formation of immune complexes in glomeruli of the kidneys. The development of lupus-like symptoms in these mice is accompanied by increased expression of CXCL13 by dendritic cells in kidneys and thymus (Ishikawa et al., J. Exp. Med. 193(12):1393-1402 (2001); Schiffer et al., J. Immun. 171:489-497 (2003)).

Neutralization of CXCL13 using an anti-CXCL13 monoclonal antibody or antigen binding fragment thereof of the invention, e.g., MAb 5261, may be used to reduce the severity of SLE through several different mechanisms, e.g., blockade of CXCL13 interaction with its receptor resulting, e.g., in interference with B and follicular B-helper T cell migration into inflamed tissues and germinal center formation.

In one embodiment, the anti-CXCL13 binding molecule, e.g., an antibody or antigen binding fragment, of the invention is used to treat or prevent arthritis, e.g., Rheumatoid Arthritis. Rheumatoid arthritis (RA) is one of the most common autoimmune diseases which affect 2-4% of people in the United States. It is a chronic, progressive, systemic inflammatory disorder characterized by fusion of synovial joints, cartilage erosion and bone destruction. In RA, T-cells, B-cells, macrophages and dendritic cells (DCs) accumulate in the synovial layer forming pannus (invasive region of synovium that erodes into cartilage and bone). Moreover, T and B cells within the synovial lesions organize into lymphoid germinal center-like structures that support autoantibody (rheumatoid factor) production and, therefore, directly contribute to the disease pathogenesis (Takemura et al., J. Immuno. 167:1072-1080 (2001); Shi et al., J. of Immuno. 166:650-655 (2001)).

Lymphoid chemokine CXCL13, produced by synovial fibroblasts, selected endothelial cells, synovial antigen-primed T helper cells and FDCs (Takemura et al. (2001); Shi et al. (2001); Manzo et al., Arthritis & Rheumatism 58(11):3377-3387 (2008)), plays a critical role in the germinal center formation in the synovial tissue, by directing CXCR5-positive lymphoid cell (primarily B cells and follicular T helper cells) migration into the inflamed synovium.

Clinically, plasma levels of CXCL13 in RA patients correlated with disease severity, as significantly higher levels of CXCL13 were found in plasma from the patients with active RA comparing to the controls and the patient with the quiescent disease (Rioja et al., Arthritis & Rheumatism 58(8):2257-2267 (2008)). In addition, CXCR5 receptor was upregulated in synovium of RA patients and present on infiltrating B and T cells and also on macrophages and endothelial cells (Schmutz et al., Arthritis Research Therapy 7:R217-R229 (2005)).

Collagen-induced arthritis (CIA) in mice and rats is a well-established model of human Rheumatoid arthritis (RA). The disease is typically induced by intradermal injection of bovine type II collagen emulsified in Complete Freund's Adjuvant (CFA) and is characterized by production of mouse collagen antibodies and, subsequently, progressive development of arthritis in the paws. Stannard et al. showed that CXCL13 neutralization with rat anti-murine CXCL13 antibodies led to significant reduction in arthritic score and the severity of joint destruction in arthritic DBA/1 mice. Stannard et al., “Neutralization of CXCL13 impacts B-cell trafficking and reduces severity of established experimental arthritis,” Presented at American College of Rheumatology 2008 Annual Scientific Meeting (2008).

Neutralization of CXCL13 using an anti-CXCL13 monoclonal antibody or antigen binding fragment thereof of the invention, e.g., MAb 5261, may be used to reduce the severity of arthritis, e.g., Rheumatoid Arthritis, through several different mechanisms, e.g., blockade of CXCL13 interaction with its receptor resulting, e.g., in interference with B and follicular B-helper T cell migration into inflamed tissues and germinal center formation. In addition, an anti-CXCL13 monoclonal antibody or antigen binding fragment thereof of the invention, e.g., MAb 5261, may be used to reduce RANKL expression and bone loss, e.g., in a subject with RANKL overexpression.

In accordance with the methods of the present invention, at least one anti-CXCL13 binding molecule, e.g., an antibody or antigen binding fragment thereof, as defined elsewhere herein is used to promote a positive therapeutic response with respect to treatment or prevention of an autoimmune disease and/or inflammatory disease. By “positive therapeutic response” with respect to an autoimmune disease and/or inflammatory disease is intended an improvement in the disease in association with the anti-inflammatory activity, anti-angiogenic activity, anti-apoptotic activity, or the like, of these antibodies, and/or an improvement in the symptoms associated with the disease. That is, an anti-proliferative effect, the prevention of further proliferation of the CXCL13-expressing cell, a reduction in the inflammatory response including but not limited to reduced secretion of inflammatory cytokines, adhesion molecules, proteases, immunoglobulins (in instances where the CXCL13 bearing cell is a B cell), combinations thereof, and the like, increased production of anti-inflammatory proteins, a reduction in the number of autoreactive cells, an increase in immune tolerance, inhibition of autoreactive cell survival, reduction in apoptosis, reduction in endothelial cell migration, increase in spontaneous monocyte migration, reduction in and/or a decrease in one or more symptoms mediated by stimulation of CXCL13-expressing cells can be observed. Such positive therapeutic responses are not limited to the route of administration and may comprise administration to the donor, the donor tissue (such as for example organ perfusion), the host, any combination thereof, and the like.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy with the anti-CXCL13 binding molecule, e.g., an antibody or antigen-binding fragment thereof, may experience the beneficial effect of an improvement in the symptoms associated with the disease.

A further embodiment of the invention is the use of an anti-CXCL13 binding molecule, e.g., antibodies or antigen binding fragments thereof, for diagnostic monitoring of protein levels in tissue as part of a clinical testing procedure, e.g., to determine the efficacy of a given treatment regimen. For example, detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

Methods of preparing and administering the anti-CXCL13 binding molecule, e.g., antibodies, or antigen-binding fragments, variants, or derivatives thereof, of the invention to a subject in need thereof are well known to or are readily determined by those skilled in the art. The route of administration of the anti-CXCL13 binding molecule, e.g, antibody, or antigen-binding fragment, variant, or derivative thereof, may be, for example, oral, parenteral, by inhalation or topical. The term parenteral as used herein includes, e.g., intravenous, intraarterial, intraperitoneal, intramuscular, subcutaneous, rectal, or vaginal administration. While all these forms of administration are clearly contemplated as being within the scope of the invention, an example of a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, anti-CXCL13 binding molecules, e.g., antibodies, or antigen-binding fragments, variants, or derivatives thereof, of the invention can be delivered directly to the site of the adverse cellular population thereby increasing the exposure of the diseased tissue to the therapeutic agent.

As discussed herein, anti-CXCL13 binding molecules, e.g., antibodies, or antigen-binding fragments, variants, or derivatives thereof, of the invention may be administered in a pharmaceutically effective amount for the in vivo treatment of CXCL13-expressing cell-mediated diseases such as certain types of cancers, autoimmune diseases, and inflammatory diseases including central nervous system (CNS) and peripheral nervous system (PNS) inflammatory diseases. In this regard, it will be appreciated that the disclosed binding molecules of the invention will be formulated so as to facilitate administration and promote stability of the active agent. In certain embodiments, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of an anti-CXCL13 binding molecule, e.g., an antibody, or antigen-binding fragment, variant, or derivative thereof, conjugated or unconjugated, shall be held to mean an amount sufficient to achieve effective binding to a target and to achieve a benefit, e.g., to ameliorate symptoms of a disease or disorder or to detect a substance or a cell.

The pharmaceutical compositions used in this invention comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. In the subject invention, pharmaceutically acceptable carriers include, but are not limited to, 0.01-0.1 M, e.g., 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as, for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences (Mack Publishing Co.) 16th ed. (1980).

Prevention of the action of microorganisms can be achieved by various antibacterial and antifungal agents, for example, parabens, chlorobutanol, phenol, ascorbic acid, thimerosal and the like. In certain cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an anti-CXCL13 antibody, or antigen-binding fragment, variant, or derivative thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in U.S. patent application Ser. No. 09/259,337. Such articles of manufacture will preferably have labels or package inserts indicating that the associated compositions are useful for treating a subject suffering from, or predisposed to a disease or disorder.

Parenteral formulations may be a single bolus dose, an infusion or a loading bolus dose followed with a maintenance dose. These compositions may be administered at specific fixed or variable intervals, e.g., once a day, or on an “as needed” basis.

Certain pharmaceutical compositions used in this invention may be orally administered in an acceptable dosage form including, e.g., capsules, tablets, aqueous suspensions or solutions. Certain pharmaceutical compositions also may be administered by nasal aerosol or inhalation. Such compositions may be prepared as solutions in saline, employing benzyl alcohol or other suitable preservatives, absorption promoters to enhance bioavailability, and/or other conventional solubilizing or dispersing agents.

The amount of an anti-CXCL13 binding molecule, e.g., antibody, or fragment, variant, or derivative thereof, that may be combined with the carrier materials to produce a single dosage form will vary depending upon the host treated and the particular mode of administration. The composition may be administered as a single dose, multiple doses or over an established period of time in an infusion. Dosage regimens also may be adjusted to provide the optimum desired response (e.g., a therapeutic or prophylactic response).

In keeping with the scope of the present disclosure, anti-CXCL13 antibodies, or antigen-binding fragments, variants, or derivatives thereof of the invention may be administered to a human or other animal in accordance with the aforementioned methods of treatment in an amount sufficient to produce a therapeutic effect. The anti-CXCL13 antibodies, or antigen-binding fragments, variants, or derivatives thereof of the invention can be administered to such human or other animal in a conventional dosage form prepared by combining an antibody of the invention, e.g., MAb 5261, with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of anti-CXCL13 binding molecules, e.g., antibodies, or antigen-binding fragments, variants, or derivatives thereof, of the invention may prove to be particularly effective.

By “therapeutically effective dose or amount” or “effective amount” is intended an amount of anti-CXCL13 binding molecule, e.g., antibody or antigen binding fragment thereof, that when administered brings about a positive therapeutic response with respect to treatment of a patient with a disease to be treated.

Therapeutically effective doses of the compositions of the present invention, for treatment of CXCL13-expressing cell-mediated diseases such as certain types of cancers, e.g., leukemia, lymphoma (e.g., MALT lymphoma), colon, breast, esophageal, stomach, and prostate cancer; autoimmune diseases, e.g., lupus, arthritis, multiple sclerosis, and inflammatory diseases including central nervous system (CNS) and peripheral nervous system (PNS) inflammatory diseases, vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

The amount of at least one anti-CXCL13 binding molecule, e.g., antibody or binding fragment thereof, to be administered is readily determined by one of ordinary skill in the art without undue experimentation given the disclosure of the present invention. Factors influencing the mode of administration and the respective amount of at least one anti-CXCL13 binding molecule, e.g., antibody, antigen-binding fragment, variant or derivative thereof include, but are not limited to, the severity of the disease, the history of the disease, and the age, height, weight, health, and physical condition of the individual undergoing therapy. Similarly, the amount of anti-CXCL13 binding molecule, e.g., antibody, or fragment, variant, or derivative thereof, to be administered will be dependent upon the mode of administration and whether the subject will undergo a single dose or multiple doses of this agent.

The present invention also provides for the use of an anti-CXCL13 binding molecule, e.g., an antibody or antigen-binding fragment, variant, or derivative thereof, in the manufacture of a medicament for treating an autoimmune disease and/or inflammatory disease, including, e.g., MS, arthritis, lupus, gastritis, an ulcer, or a cancer.

The invention further provides a diagnostic method useful during diagnosis of CXCL13-expressing cell-mediated diseases such as certain types of cancers and inflammatory diseases including autoimmune diseases, which involves measuring the expression level of CXCL13 protein or transcript in tissue or other cells or body fluid from an individual and comparing the measured expression level with a standard CXCL13 expression level in normal tissue or body fluid, whereby an increase in the expression level compared to the standard is indicative of a disorder. In certain embodiments, the anti-CXCL13 antibodies of the invention or antigen-binding fragments, variants, and derivatives thereof, e.g., MAb MAb 5261, MAb 5378, MAb 5080, MAb 1476, 3D2, or 3C9, are used in diagnosis of cancer, multiple sclerosis, arthritis, or lupus.

The anti-CXCL13 antibodies of the invention and antigen-binding fragments, variants, and derivatives thereof, can be used to assay CXCL13 protein levels in a biological sample using classical immunohistological methods known to those of skill in the art (e.g., see Jalkanen, et al., J. Cell. Biol. 101:976-985 (1985); Jalkanen et al., J. Cell Biol. 105:3087-3096 (1987)). Other antibody-based methods useful for detecting CXCL13 protein expression include immunoassays, such as the enzyme linked immunosorbent assay (ELISA), immunoprecipitation, or Western blotting. Suitable assays are described in more detail elsewhere herein.

By “assaying the expression level of CXCL13 polypeptide” is intended qualitatively or quantitatively measuring or estimating the level of CXCL13 polypeptide in a first biological sample either directly (e.g., by determining or estimating absolute protein level) or relatively (e.g., by comparing to the disease associated polypeptide level in a second biological sample). In one embodiment, the CXCL13 polypeptide expression level in the first biological sample is measured or estimated and compared to a standard CXCL13 polypeptide level, the standard being taken from a second biological sample obtained from an individual not having the disorder or being determined by averaging levels from a population of individuals not having the disorder. As will be appreciated in the art, once the “standard” CXCL13 polypeptide level is known, it can be used repeatedly as a standard for comparison.

By “biological sample” is intended any biological sample obtained from an individual, cell line, tissue culture, or other source of cells potentially expressing CXCL13. Methods for obtaining tissue biopsies and body fluids from mammals are well known in the art.

Anti-CXCL13 antibodies, or antigen-binding fragments, variants, or derivatives thereof of the invention may be assayed for immunospecific binding by any method known in the art. The immunoassays that can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, (1994) Current Protocols in Molecular Biology (John Wiley & Sons, Inc., NY) Vol. 1, which is incorporated by reference herein in its entirety).

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

The binding activity of a given lot of anti-CXCL13 antibody, or antigen-binding fragment, variant, or derivative thereof may be determined according to well known methods. Those skilled in the art will be able to determine operative and optimal assay conditions for each determination by employing routine experimentation.

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities.

Surface plasmon reasonance (SPR) as performed on BIACORE® offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BlAapplications Handbook, version AB (reprinted 1998), BIACORE® code No. BR-1001-86; BlAtechnology Handbook, version AB (reprinted 1998), BIACORE® code No. BR-1001-84. SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIACORE® measurements monitor interactions directly as they happen. The technique is well suited to determination of kinetic parameters. Comparative affinity ranking is simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=K_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIACORE® investigations. The range of affinities measurable by this technique is very broad ranging from mM to pM.

Epitope specificity is an important characteristic of a monoclonal antibody. Epitope mapping with BIACORE®, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BIACORE® are straightforward to carry out.

For example, one can use a capture molecule to bind the first Mab, followed by addition of antigen and second MAb sequentially. The sensorgrams will reveal: (1) how much of the antigen binds to first Mab, (2) to what extent the second MAb binds to the surface-attached antigen, (3) if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides which interfere with binding of a given MAb are assumed to be structurally related to the epitope defined by that MAb.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described are generally followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevere, Amsterdam); Goldsby et al., eds. (2000) Kuby Immunnology (4th ed.; H. Freemand & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlan); Sambrook and Russell (2001) Molecular Cloning: A Laboratory Manual (Cold Spring Harbor Press); Lewin (2003) Genes VIII (Prentice Hall 2003); Harlow and Lane (1988) Antibodies: A Laboratory Manual (Cold Spring Harbor Press); Dieffenbach and Dveksler (2003) PCR Primer (Cold Spring Harbor Press).

It is to be noted that the term “a” or “an” entity refers to one or more of that entity; for example, “an anti-CXCL13 antibody” is understood to represent one or more anti-CXCL13 antibodies. As such, the terms “a” (or “an”), “one or more,” and “at least one” can be used interchangeably herein.

All technical and scientific terms used herein have the same meaning. Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller rangers is also encompassed within the invention, subject to any specifically excluded limit in the stated range. Where the stated range includes one or both of the limits, ranges excluding either or both of those included limits are also included in the invention.

The following examples are offered by way of illustration and not by way of limitation.

### EXPERIMENTAL

### Example 1

### Selection and Characterization of Mouse Antibodies Specific for Human CXCL13

Hybridoma Generation.

Swiss Webster mice were immunized with Keyhole limpet hemocyanin (KLH)-conjugated human CXCL13. After three immunizations, spleen was harvested from the mouse with the highest anti-CXCL13 titer and hybridomas were generated by fusion of spleen cells with SP2/0 myeloma cells using standard procedures.

Hybridoma clones were screened by ELISA for binding to human and mouse CXCL13 protein. ELISA plates were coated with about 100 nM of human (Peprotech: #300-47) or mouse (Peprotech: #250-24) CXCL13 protein overnight at room temperature (RT). After the plates were washed and blocked, dilutions of standard anti-CXCL13 antibodies (R&D Systems: rat anti-mouse MAb 470 and mouse anti-human MAb 801) or hybridoma supernatant were added and incubated for 1 hour at RT. The plates were washed and secondary antibodies (goat anti-mouse IgG-HRP for hybridoma supernatant and MAb 801; donkey anti-rat IgG-HRP for MAb 470) were added and incubated for 1 hour at RT. The plates were washed and developed with tetramethylbenzidine (TMB) substrate reagents A and B (BD Biosciences: #555214) mixed at equal volumes for 15 minutes in the dark and read at 450/570 wavelengths. Two positive hybridoma clones, labelled “3D2” and “3C9”, both mouse IgG1 antibodies, were selected for further characterization.

Specificity of the hybridoma-produced mouse anti-human antibodies, 3D2 and 3C9, was assessed by ELISA (as described above) on a panel that included recombinant chemokines (Peprotech: mouse and human CXCL13, human IL-8/CXCL8 (#200-08), human IP-10/CXCL10 (#200-10), human MIG/CXCL9 (#300-26), human SDF-1alpha/CXCL12 (#300-28A) and cynomolgus monkey CXCL13) as well as several non-specific control antigens (recombinant human C35, streptavidin (Thermo: #21122), bovine serum albumin (BSA) (SeraCore: #AP-4510-01)), human serum albumin (HAS) (Sigma: #A8763), insulin (Gibco: #12585-014), and hemoglobin (Sigma: #H7379). Commercial antibodies MAb 470 and MAb 801 (R&D Systems) were used as positive controls for mouse and human/cynomolgus monkey CXCL13 binding, respectively.

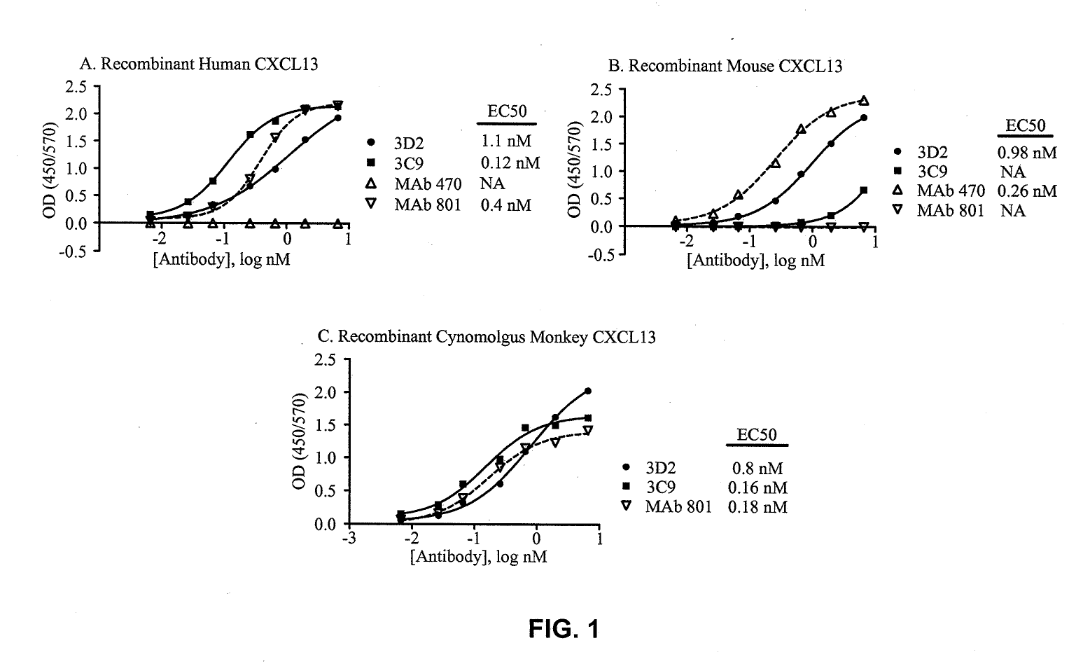

3D2 and 3C9 were shown to specifically bind CXCL13. Both 3D2 and 3C9 clones demonstrated multi-species CXCL13 specificity as they bound to recombinant human, mouse and cynomolgus monkey CXCL13 (FIGS. 1A-1C). FIG. 1 shows results from duplicate measurements for at least three experiments. 3D2 antibody was shown to have strong binding to recombinant human, mouse and monkey CXCL13. In particular, 3D2 had stronger binding to recombinant mouse CXCL13 compared to 3C9 and MAb 801. 3D2 was further characterized in vitro and in vivo (results shown below). The 3D2 antibody was also used as a prototype for generation of a chimeric and humanized CXCL13 antibodies (results shown below). 3C9 antibody was shown to have the strongest binding to recombinant human CXCL13 compared to 3D2, MAb 470, and MAb 801. 3C9 and 3D2 were used as reagents in Bioassay development (e.g., Epitope Competition ELISA, described below).

3D2 Affinity Measurements by BIACORE®.

The affinity of 3D2, 3C9, MAb 801 and MAb 470 for recombinant human and mouse CXCL13 was measured by BIACORE®. Chemokines were immobilized on a C1 chip in Acetate buffer (pH=5) with human CXCL13 at 1 μg/ml, mouse CXCL13 at 0.3 μg/ml, and negative control SDF-1α at 0.5 μg/ml. 3D2 and 3C9 were diluted in HBS-EP buffer by two-fold from 100 nM to 0.78 nM and from 38 nM to 0.594 nm. MAb 801 and MAB 470 were diluted by two-fold from 50 nM to 0.78 nM and from 19 nM to 0.594 nm. The results showed that the affinity measurement (nM) for 3D2 on human and mouse CXCL13 was significantly lower than that of commercial antibodies MAb 801 and MAb 470, respectively. The results are shown in Table 2.

TABLE 2
Affinity Measurements¹

| | | Affinity, nM | |
|---|---|---|---|
| Antibody | Fc | Human CXCL13 | Mouse CXCL13 |
| 3D2 | Mouse IgG1 | 12.9 | 159.3 |
| 3C9 | Mouse IgG1 | 2.5 | NB |
| MAb 801 | Mouse IgG1 | 1.3 | NB |
| MAb 470 | Rat IgG2a | NB | 5.4 |

¹affinities are for recombinant human and mouse CXCL13;
NB = no binding

3D2 Epitope Mapping.

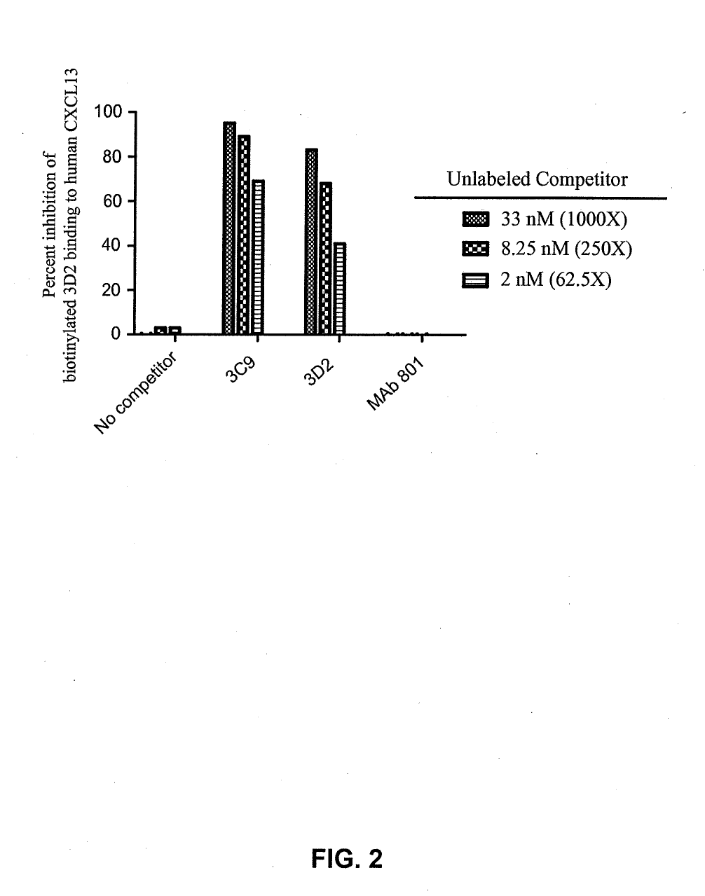

An epitope mapping study was conducted using Epitope Competition ELISA. Plates were coated with 100 nM recombinant human CXCL13 and incubated with 0.033 nM biotinylated 3D2 prior to addition of competing unlabeled antibodies at various concentrations in excess of the amount of 3D2. The results from a representative experiment are shown in FIG. 2. The results show that 3C9 competes with 3D2 for binding to human CXCL13, but MAb 801 did not compete with 3D2 for binding to human CXCL13.

3D2 Binding to Native CXCL13.

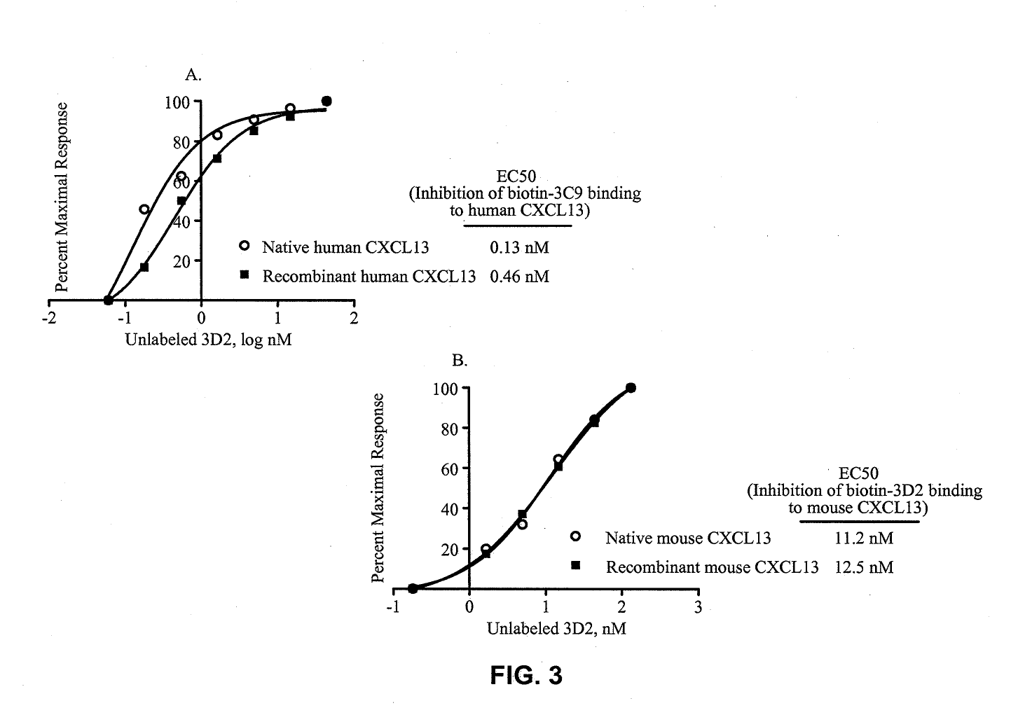

Capture Epitope Competition ELISA was used to assess 3D2 binding to native human and mouse CXCL13. In this assay, native human CXCL13 was obtained from supernatants collected from human IFN-gamma-stimulated THP-1 cells. Human CXCL13 (1:4 dilution of THP1 supernatant or 0.097 nM (1 ng/ml) rhuCXCL13) was captured with 6.6 nM MAb 801 and detected with 0.66 nM biotin-3C9. For appropriate antigen presentation, the chemokine from tissue culture supernatant (or recombinant human CXCL13 which was used as a control) was captured on the ELISA plate by MAb 801. The plate was then incubated with biotinylated 3C9 in the presence of various amounts of unlabeled 3D2. The competition for binding to human CXCL13 that resulted in reduced binding of biotinylated 3C9 was detected by Streptavidin-HRP. As evident from FIG. 3A, 3D2 strongly bound to both native and recombinant human CXCL13 producing statistically similar EC50 values.

Mouse CXCL13-rich organ extracts from TNF-alpha transgenic mice were used as sources of native mouse CXCL13. Mouse CXCL13 (1:40 dilution of TNF-Tg organ extract or 0.5 nM rmuCXCL13) was captured with 33 nM MAb 470 and detected with 3.3 nM biotin-3D2. The chemokine from the extracts (and recombinant mouse CXCL13 which was used as a control) was captured on the ELISA plate by MAb 470. The plate was then incubated with biotinylated 3D2 in the presence of various amounts of unlabeled 3D2. The competition for the binding to mouse CXCL13 that resulted in reduced binding of biotinylated 3D2 was then detected by Streptavidin-HRP. As can be seen from FIG. 3B, 3D2 was able to compete off the biotinylated version of itself and bind to both native and recombinant mouse CXCL13 with equal potency.

For both FIGS. 3A and 3B, each data point represents an average of duplicate measurements from one of at least three independent experiments. Curves were fitted using four-parameter sigmoidal curve fit (R²=0.99). Differences in EC50 values were analyzed by unpaired t-test and were not significant (P>0.05). These results show that the mouse anti-human 3D2 antibody bound not only the recombinant chemokine against which it had been generated, but also native human and mouse chemokines.

### Example 2

### Anti-CXCL13 Antibody Inhibition of Human B-Cell Migration

Inhibition of CXCL13 function, e.g., B-cell migration, was evaluated using established models that simulate B-cell migration in both human and mouse systems. Migration towards a non-CXCL13 chemokine, SDF-1α (a.k.a. CXCL12), was used as a control to confirm specificity of anti-CXCL13 antibody on inhibition of B-cell migration. Thus, anti-CXCL13 antibodies were tested for inhibition of human CXCL13-induced migration and the absence of an effect on SDF-1α-induced migration.

Inhibition of Human B-Cell Migration Towards Human CXCL13.

The effect of 3D2, 3C9, and MAb 801 on human CXCL13-induced B-cell migration was tested.

Two clonal cell lines, human pre-B-697-hCXCR5 and human pre-B-697-hCXCR4, were used to assess the effects of anti-CXCL13 antibodies on recombinant human CXCL13-dependent migration and recombinant human SDF-1α-dependent migration, respectively. Transwell tissue culture-treated plates with 8 μm pore size and diameter of 6.5 mm (Corning Costar: 3422) were used. Human pre-B-697-hCXCR5 cells were used for rhCXCL13-induced migration, and human pre-B-697-hCXCR4 for were used for rhSDF-1-induced migration (negative control). Cells were resuspended in chemotaxis buffer ((RPMI 1640 with 1-glutamine, 10 mM HEPES, PennStrep and 0.5% BSA) pre-warmed to RT) at 5×10⁶/ml and returned to 37° C. for 30 minutes. Diluted chemokine (97 nm (1 μg/ml) rhCXCL13 or 12.5 nM (0.1 μg/ml) rhSDF-1α in chemotaxis buffer) +/− antibodies were added into the lower chamber at 590 μl/well and pre-incubated for 30 minutes at RT. The cells were added at 100 μl (5×10⁵) cells/upper chamber. Plates were incubated overnight at 37° C. Inserts were then removed and Alamar blue was added at 60 μl per well and the plates were incubated at 37° C. for 4 hours. Fluorescence was measured at wavelengths 530 nm and 590 nm.

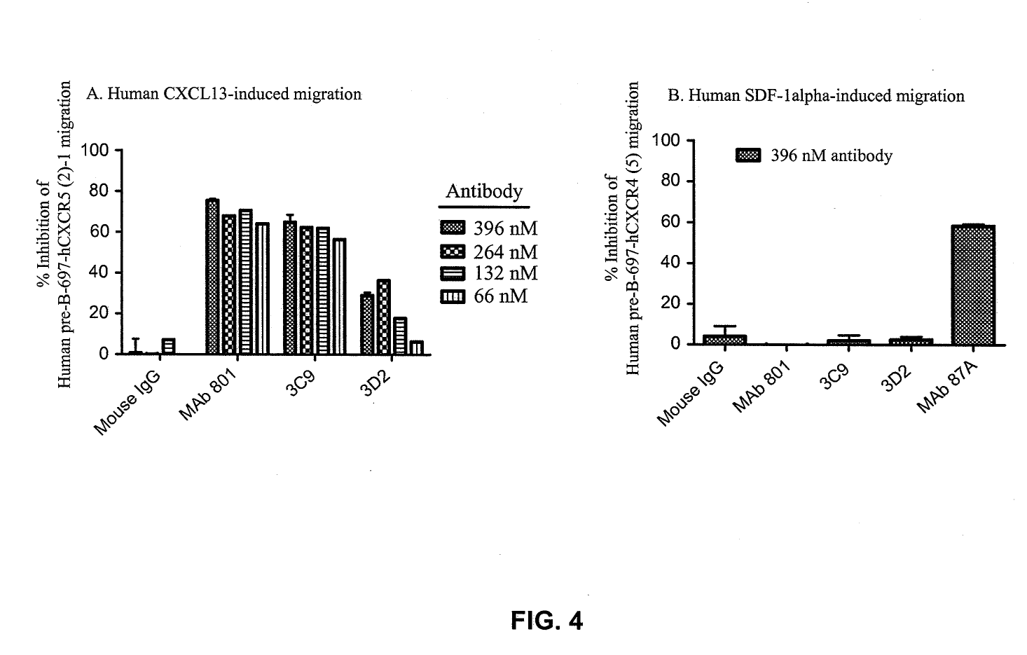

Migration index was calculated for each condition as follows: ((Migration Index [Isotype control]−Migration Index [antibody])*100)/(Migration Index [Isotype control]). Percent migration inhibition was plotted against log[nM antibody] to obtain a titration curve using Graphpad Prism 5. The results for human CXCL13-induced migration are shown in FIG. 4A. The results are presented as mean of two hCXCL13-induced migration and three hSDF-1-induced migration independent experiments +/−SEM.

Differences in the degrees of inhibition of human CXCL13-induced migration among 3D2, 3C9 and MAb 801 corresponded to the differences in affinities for human CXCL13 (see Table 2 above). Thus, the antibody with the lowest affinity for the chemokine (3D2) appeared to be the weakest inhibitor of human pre-B-hCXCR5 chemotaxis, whereas antibodies with higher, nearly identical affinities (MAb 801 and 3C9) resulted in a high percent inhibition of cell migration (FIG. 4A). None of the anti-human CXCL13 antibodies (3D2, 3C9, or MAb 801) produced any effect on human SDF-1α-mediated chemotaxis of human pre-B-hCXCR4 (5) cells (FIG. 4B). Whereas, positive control goat anti-human SDF-1α antibody (MAb 87A) strongly inhibited SDF-1α-dependent migration.

Inhibition of Mouse CXCL13-Dependent Migration of Mouse Splenocytes.

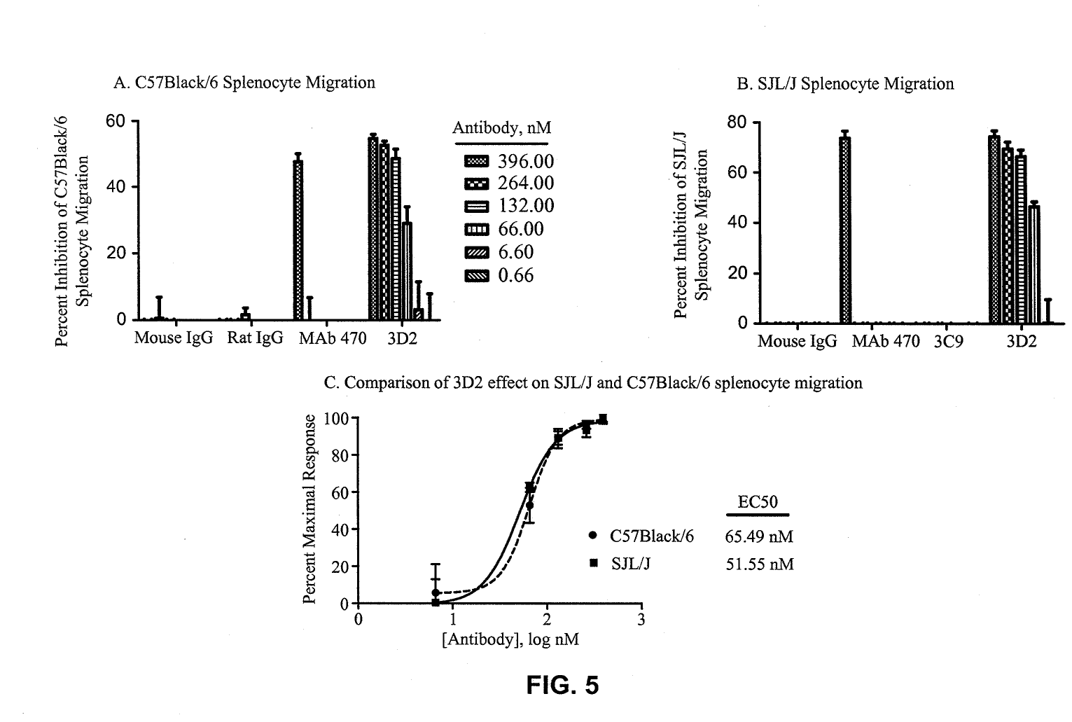

Anti-CXCL13 antibodies were tested for their ability to inhibit recombinant mouse CXCL13-mediated chemotaxis of mouse spleenocytes (obtained from mechanically dissociated spleens). The assay was performed using essentially the same protocol as described above for the human CXCL13-dependent B-cell migration assay with minor changes including using 485 nM (5 μg/ml) rmuCXCL13, using transwell plates with smaller pore size (i.e., Transwell tissue culture treated plates with 5 μm pore size and diameter of 6.5 mm (Corning Costar: #3421)), and using higher amounts of cells (10⁶) per well. The effect of the tested antibodies on migration of spleenocytes from two different strains of mice, C57/BL6 and SJL are shown in FIGS. 5A and 5B, respectively. MAb 470 was used as a positive control. Rat and mouse IgG as well as human CXCL13-specific mouse antibody 3C9 were included as a negative controls. As shown in FIGS. 5A and 5B, the patterns of inhibition were different between MAb 470 and 3D2. In particular, 3D2 inhibited chemotaxis in a titratable manner, whereas, the effect of MAb 470 was only apparent at the highest antibody concentration of 396 nM. Data comparing the effect of 3D2 on C57Black/6 and SJL/J migration were analyzed by unpaired t-test which produced P value >0.05 indicating an absence of significant differences between two curves. Curves were fitted using four-parameter sigmoidal curve fit (R²=0.99). A comparison of 3D2 effect on SJL/J and C57Black/6 splenocyte migration is shown in FIG. 5C. No significant differences in 3D2 inhibitory profiles between two mouse strains was shown.

### Example 3

### Anti-CXCL13 Antibody Inhibition of CXCL13-Mediated Endocytosis of Human CXCR5

Inhibition of CXCL13 chemokine function, e.g., CXCL13-mediated endocytosis of CXCR5 receptor, with anti-CXCL13 antibodies was evaluated using an established model for human CXCL13-mediated endocytosis of human CXCR5 receptor (Burke et al., Blood 110:2216-3325 (2007)).

Inhibition of CXCL13-Mediated Endocytosis of Human CXCR5 Receptor.

Binding of a chemokine to its chemokine receptor leads to internalization of the ligand-receptor complex with subsequent activation of intracellular signaling cascade (Neel et al., Chemokine and Growth Factor Reviews 16:637-658 (2005)). The flow-based method described in Burkle et al. was adapted to determine the ability of 3D2 to inhibit CXCL13-mediated CXCR5 receptor internalization in both human and mouse cells. For the human CXCL13-mediated endocytosis, hCXCL13 was combined with 3D2, MAb 470 or Mouse IgG (at concentrations 0, 33, 66, 132, 264, and 528 nM) and incubated overnight at 4° C. The next day, the cells were resuspended in diluents (RPMI+0.5% BSA) at 10⁷ cells/ml. The cells were pre-blocked with 10 μg/ml anti-human Fc block for 15 min at 37° C. The cells were then incubated with the CXCL13/antibody mix (50 μl cells:50 μl mix) for 2 hours at 37° C. The cells were then stained with anti-human CXCR5 antibody (BDPharmingen: #558113) for 30 minutes at 4° C. and analyzed by flow cytometry. Similarly, mouse CXCL13-mediated endocytosis was assayed using mCXCL13 combined with 3D2 or Mouse IgG (at concentrations 0, 20, 59, 198, and 528 nM). Inhibition of endocytosis was calculated as follows: % Inhibition=100−[100*(0 CXCL13−geomean)/(0 CXCL13−0 mAB)].

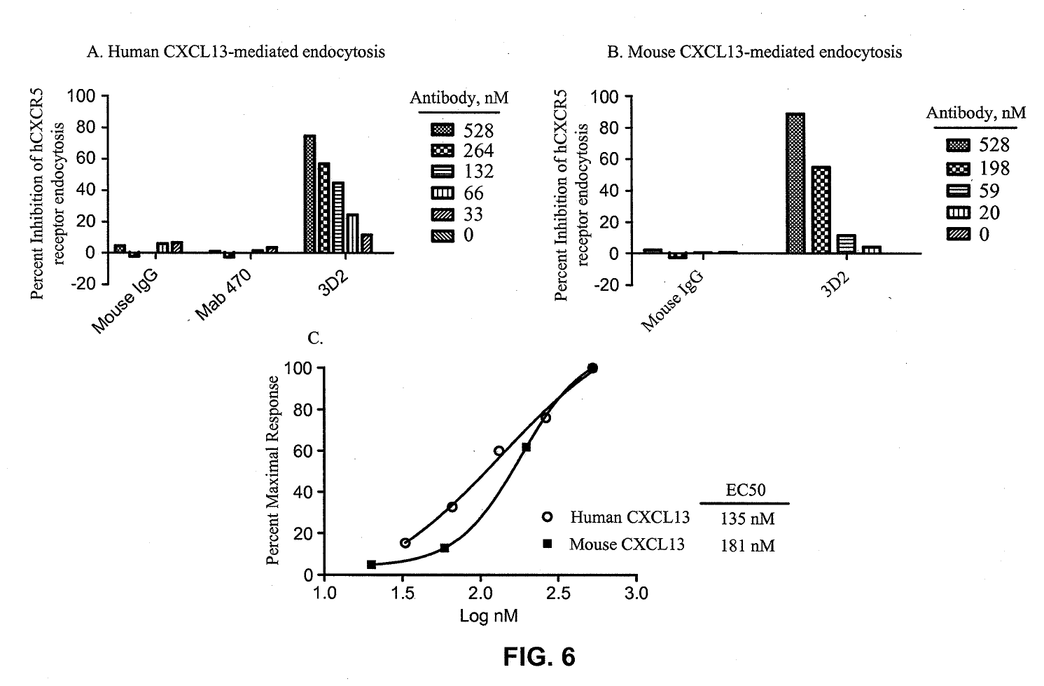

FIG. 6 shows data from representative human and mouse CXCL13 experiments. FIG. 6A shows the effect of 3D2 antibody on human CXCR5 receptor expression on the surface of human pre-B-697-hCXCR5 cells treated with 485 nM (5 μg/ml) of human CXCL13. FIG. 6B shows 3D2-mediated inhibition of mouse CXCR5 receptor internalization in Wehi-231 cells pre-incubated with 1000 nM (10 μg/ml) of mouse CXCL13. In both cases 3D2 efficiently and in a titratable manner interfered with the CXCL13-induced down regulation of CXCR5 receptors. FIG. 6C shows EC50 values which were calculated from sigmoidal dose response curves (shown on the graph) with R2 values equal to 1 (mouse endocytosis) and 0.994 (human endocytosis). The data comparing 3D2 effect on human and mouse receptor endocytosis were analyzed by unpaired t-test which produced a P value >0.05 indicating absence of significant differences between the human CXCL13 and mouse CXCL13 curves.

### Example 4

### Evaluation of Anti-CXCL13 Antibodies in Mouse Disease Models for Multiple Sclerosis

Murine Model of Multiple Sclerosis.

Experimental Autoimmune Encephalomyelitis (EAE) is a widely accepted animal model of multiple sclerosis. EAE is a demyelinating disease of the CNS that progressively results in escalating degrees of ascending paralysis with inflammation primarily targeting the spinal cord. The disease can assume an acute, chronic or relapsing-remitting course that is dependent upon the method of induction and type of animal used. Thus, EAE can be induced with the components of the CNS, peptides (active induction) and also via cell transfer from one animal to another (passive induction). Complete Freund's Adjuvant (CFA) is used with the extracts or peptides and is often used with pertussis toxin.

RR-EAE-1: 3D2 Effect on Relapsing-Remitting EAE in SJL Mice.

3D2 antibody was tested using an active immunization model of EAE. In the “RR-EAE-1” study, relapsing-remitting (RR) disease was induced in SJL/J mice by subcutaneous immunization with proteolipid protein (PLP)139_{—}151 peptide epitope (HSLGKWLGHPDKF; SEQ ID NO: 1) in 1 mg/ml CFA enhanced with 5 mg of heat-inactivated Mycobacterium tuberculosis strain H37RA. The study included the following treatment groups:
A. Control (mouse IgG) starting at Day 0
B. 3D2 starting at Day 0
C. 3D2 starting at score ≧1

The mice were given intraperitoneal injections of 0.3 mg (15 mg/kg) of antibody twice per week. The treatment started at Day 0 for groups A and B and at the clinical score of ≧1 for group C (the scoring system is described in Table 3 below).

TABLE 3
Summary of Evaluation of the EAE Clinical Signs

| Score | Signs | Description |
|---|---|---|
| 0 | Normal behavior | No neurological signs. |
| 1 | Distal limp tail | The distal part of the tail is limp and |
| | | droopy. |
| 1.5 | Complete limp tail | The whole tail is loose and droopy. |
| 2 | Righting reflex | Animal has difficulties rolling onto his |
| | | feet when laid on its back. |
| 3 | Ataxia | Wobbly walk—when the mouse walks |
| | | the hind legs are unsteady. |
| 4 | Early paralysis | The mouse has difficulties standing on |
| | | its hind legs, but still has remnants of |
| | | movement. |
| 5 | Full paralysis | The mouse can't move its legs at all, it |
| | | looks thinner and emaciated. |
| 6 | Moribund/Death | |

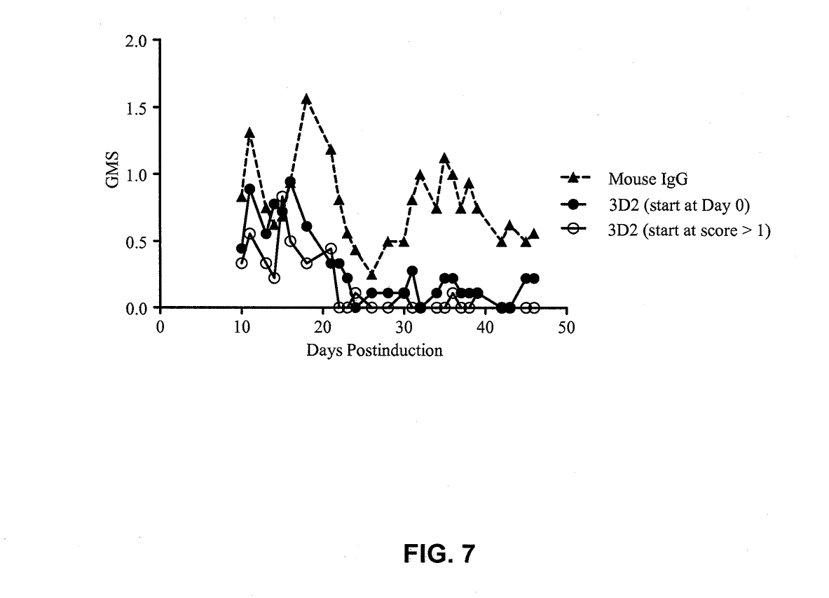

As shown in FIG. 7, treatment with 3D2 resulted in an amelioration of disease. Each data point represents a mean of scores taken from 9 mice. Group means (GMS) were compared by using one-way ANOVA followed by Bonferroni's multiple comparison post test. Statistically significant differences were observed between the control group (mouse IgG) and each 3D2 treated group (P<0.05), but not between two 3D2 treated groups (P>0.05). The disease attenuation was observed even when treatment did not begin until mice had demonstrated active disease (Group C).

RR-EAE-2: 3D2 Effect on Relapsing-Remitting EAE in SJL Mice.

A second study (“RR-EAE-2”) was done using pertussis toxin in the induction protocol to test 3D2 in a more severe disease model. For this second relapsing-remitting EAE study, SJL/J mice were subcutaneously immunized with PLP₁₃₉₋₁₅₁ in 1 mg/ml CFA enhanced with 5 mg of heat-inactivated Mycobacterium tuberculosis strain H37RA, and one-hundred nanograms of Pertussis toxin was administered intraperitonealy on Days 0 and 2 post-immunization. Treatment included bi-weekly intraperitoneal injections of 0.3 mg (15 mg/kg) antibody separated into the following groups:
A. Control (mouse IgG) starting at Day 0
B. 3D2 starting at Day 0
C. 3D2 starting at Day 7
D. 3D2 starting at EAE onset (score ≧2)

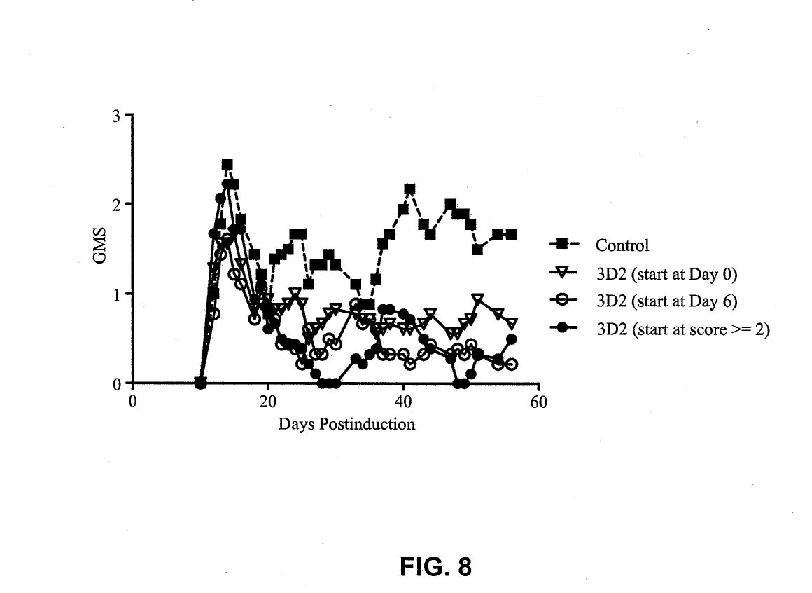

The results for RR-EAE-2 are shown in FIG. 8. Each data point represents a mean of scores taken from 9 mice. Group means were compared by using one-way ANOVA followed by Bonferroni's multiple comparison post test. Statistically significant differences were observed between control (mouse IgG) and each 3D2 treated group (P<0.05), but not among the three 3D2 treated groups (P>0.05). Again, treatment with 3D2 had a statistically significant effect on the severity of the disease, even when the treatment was started at the onset of the EAE symptoms, score ≧2 (Group D).

### Example 5

### Evaluation of Anti-CXCL13 Antibodies in Mouse Disease Model for Lupus

Murine Model of Systemic Lupus Erythematosus (SLE).

SLE is an autoimmune disease that involves multiple organs and is characterized by the spontaneous formation of ectopic germinal centers and autoantibody production against a number of nuclear antigens. The effect of anti-CXCL13 3D2 antibody was tested in a murine model of lupus. Lupus-prone New Zealand Black X New Zealand White F1 (NZB/NZWF1) mice spontaneously develop high-titer anti-dsDNA antibodies and severe proliferative glomerulonephritis caused by formation of immune complexes in glomeruli of the kidneys.

SLE-1: Treatment of Advanced Disease.

In study “SLE-1,” treatment started in twenty-four to thirty-week old NZB/NZWF1 mice with proteinuria of ≧2 (proteinuria scoring system is described in Table 4 below) and the treatment was continued for eight weeks. The treatment included bi-weekly intraperitoneal injections of 0.3 mg (15 mg/kg) of 3D2 or mouse IgG (control).

TABLE 4
Proteinuria Scores

| Proteinuria score | [Protein] in urine, mg/dl |
|---|---|
| 1+ | 30 |
| 2+ | 100 |
| 3+ | 300 |

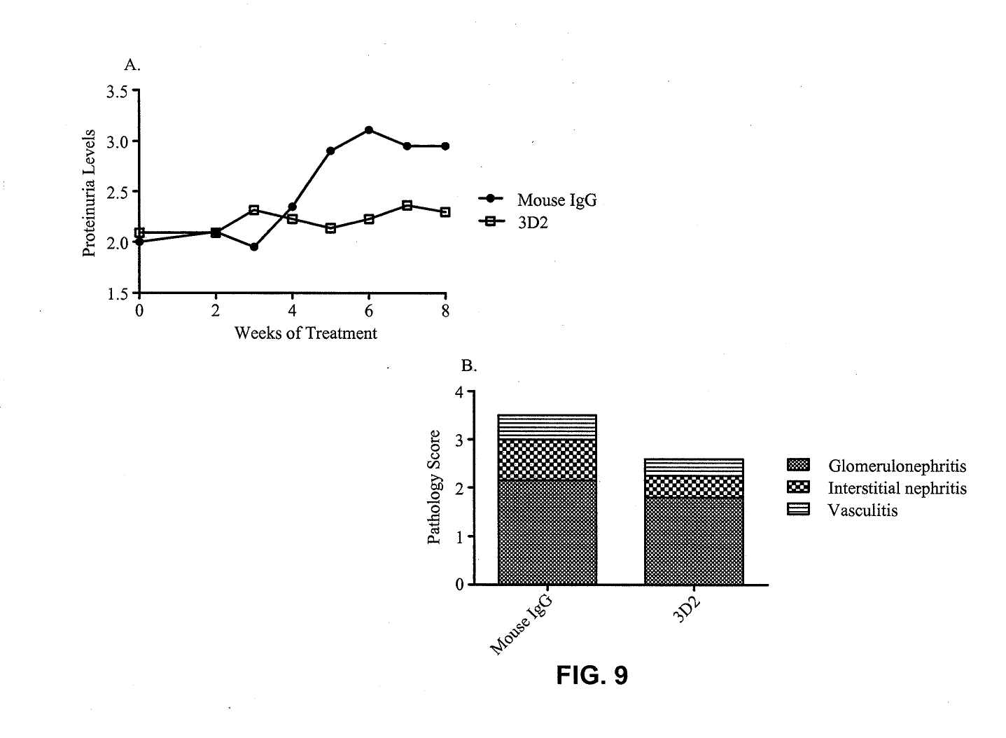

As shown in FIG. 9A, treatment with 3D2 halted progression of proteinuria. Histological analysis of kidneys using a well-defined scoring system (Table 5) also showed a beneficial effect of anti-CXCL13 treatment as the glomerulonephritis (GN), interstitual nephritis (IN), and vasculitis (VI) pathology scores were lower in the 3D2-treated group compared to control (mouse IgG). See FIG. 9B.

TABLE 5
Kidney Pathology Scores

| Scores | Glomerulonephritis | Interstitial Nephritis | Vessels |
|---|---|---|---|
| 0-1+ | Focal, mild or early | Occasional, focal or | Occasional |
| | proliferative | small pockets of | perivascular |
| | | MNC (10-15 cells) | infiltrate |
| 1-2+ | Moderate or definite | Focal infiltrates | Several foci of |
| | proliferative; | (15-30 cells) | perivascular |
| | increased matrix | | infiltrate; |
| | | | no necrosis |
| 2-3+ | Diffuse and focal or | Multifocal extensive | Multifocal |
| | diffuse proliferative | infiltrates; with | perivascular; |
| | | necrosis | more extensive; |
| | | | +/− necrosis (3+) |
| 3-4+ | Severe diffuse | Severe disease with | Multifocal or |
| | proliferative, with | extensive necrosis | diffuse; extensive |
| | crescent/sclerosis | | with necrosis |

For proteinurea scores (FIG. 9A) and kidney pathology scores (FIG. 9B), each data point represented the mean of ten measurements. No statistically significant differences were observed (P>0.05) in two-way ANOVA followed by Bonferroni's multiple comparison post test to identify statistically significant differences (P<0.05).

SLE-2: Prevention Trial in Mice with Early Disease (Post-Autoantibody Induction, but Before Significant Proteinuria).

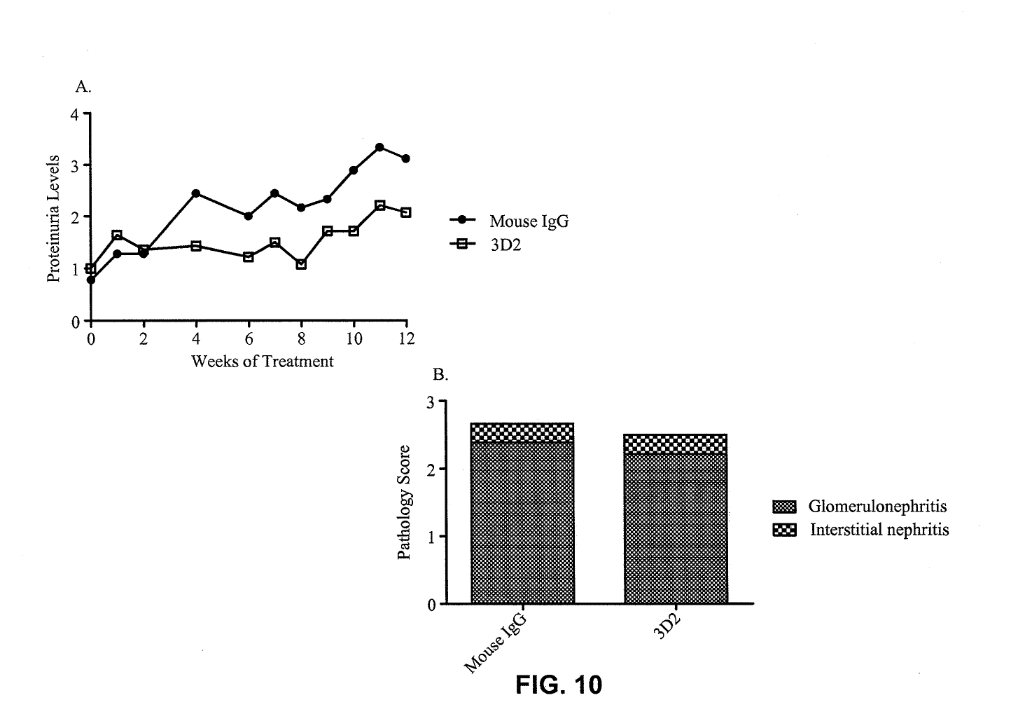

In study “SLE-2,” the treatment started in twenty-week-old NZB/NZWF1 mice and continued for twelve weeks. The treatment included bi-weekly intraperitoneal injections of 0.3 mg (15 mg/kg) of either 3D2 or mouse IgG (control). As shown in FIG. 10A, treatment with 3D2 resulted in statistically significant inhibition of the progression of proteinuria, particularly during the first eight weeks of treatment. After eight weeks, zero out of seven (0%) mice in the 3D2 treatment group and four out of nine (44%) mice in the control group had >2+proteinuria score. At the end of twelve weeks of treatment, mean urine protein was 2.1+/−0.2 with 3D2 treatment vs. 3.1+/−0.15 with mouse IgG (control) antibody.

Kidney pathology scores were also measured in mice from the 3D2-treated group and mouse IgG-treated group. A summary of the glomerulonephritis (GN) and interstitual nephritis (IN) pathology scores is shown in FIG. 10B.

Proteinuria levels (FIG. 10A) and kidney pathology scores (FIG. 10B) were measured in 7 mice from 3D2-treated group and 9 mice from mouse IgG-treated group. Proteinurea scores were significantly different between groups (P=0.0042; two-way ANOVA with Bonferroni's multiple comparison test). Kidney pathology scores were not significantly different (P>0.05). Although, mean pathology scores were not significantly different, there was histologic evidence of severe kidney disease in two out of seven (29%) mice in the 3D2 treatment group, while four out of nine (44%) mice in the control group showed evidence of severe disease. It was noted that blockade of CXCL13 by 3D2 did not prevent the development of autoantibodies (data not shown).

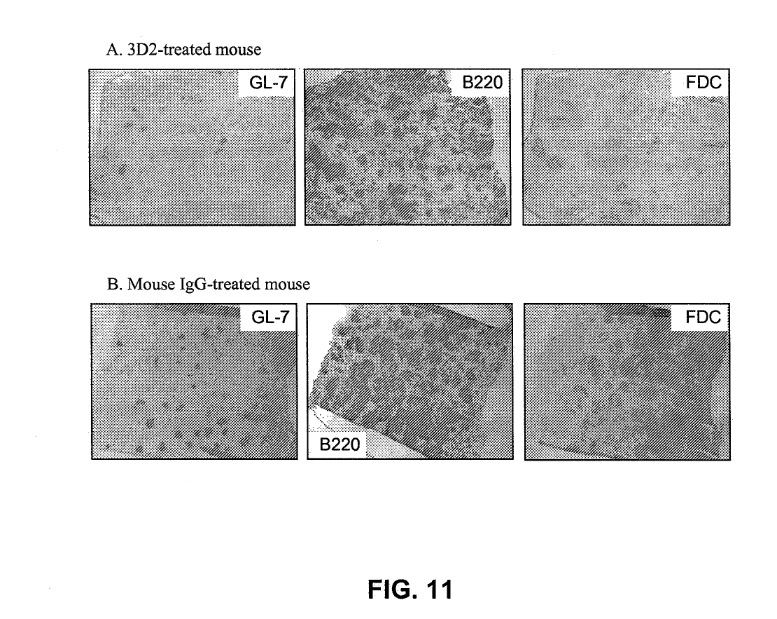

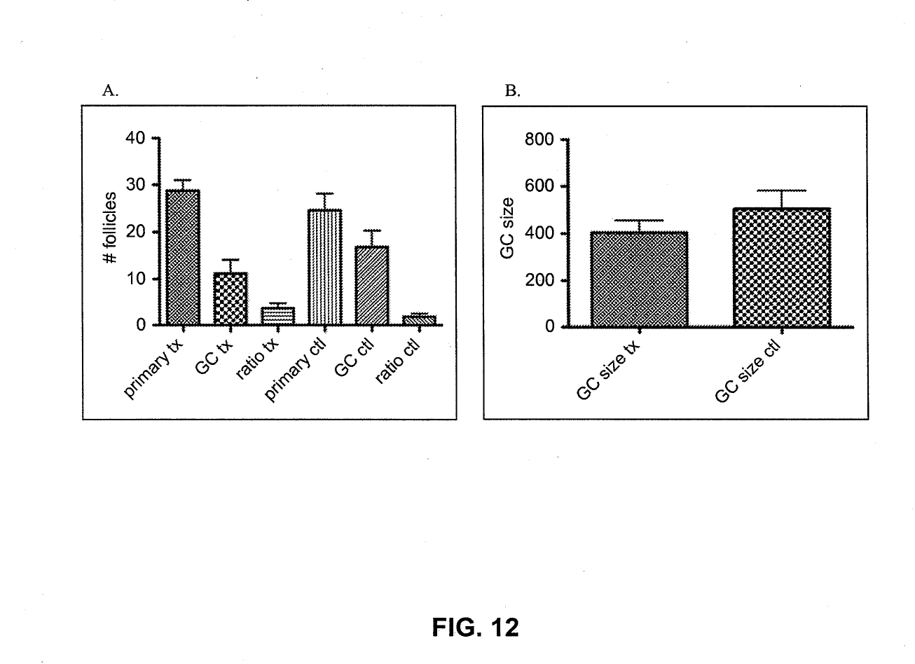

The effect of 3D2 treatment on the number of Germinal Centers (GC) and primary follicles in the spleen of lupus mice was evaluated. Spleen sections were stained with GL-7 (GC stain), B220 antibody (B cell marker), or antibody against follicular dendritic cells (FDCs) from 3D2-treated and mouse IgG-treated (control) NZB/NZWF1 mice. The effect of CXCL13 inhibition on splenic lymphoid architecture is shown in FIGS. 11A-B. The primary follicles remained intact. Mice treated with 3D2 exhibited a significant decrease in size and frequency of spontaneous germinal centers (GC) in the spleen. Mice treated with 3D2 (“tx”) showed a trend towards decreased numbers of GCs when expressed as a ratio of primary: secondary (GC) follicles (p=0.19) (FIG. 12A) and a significant decrease in GC size (p=0.03) (FIG. 12B). Values are shown as mean +/−SEM with 5 mice per group.

The above described SLE results shows that CXCL13 inhibition by 3D2 antibody leads to decreased nephritis in the NZB/NZWF1 mouse model of lupus, particularly at earlier stages of disease, and may affect splenic architecture.

### Example 6

### Preparation of Chimeric and Humanized Anti-CXCL13 Monoclonal Antibodies

Isolation of 3D2 Hybridoma V Genes and Cloning of Chimeric 3D2 Antibody.

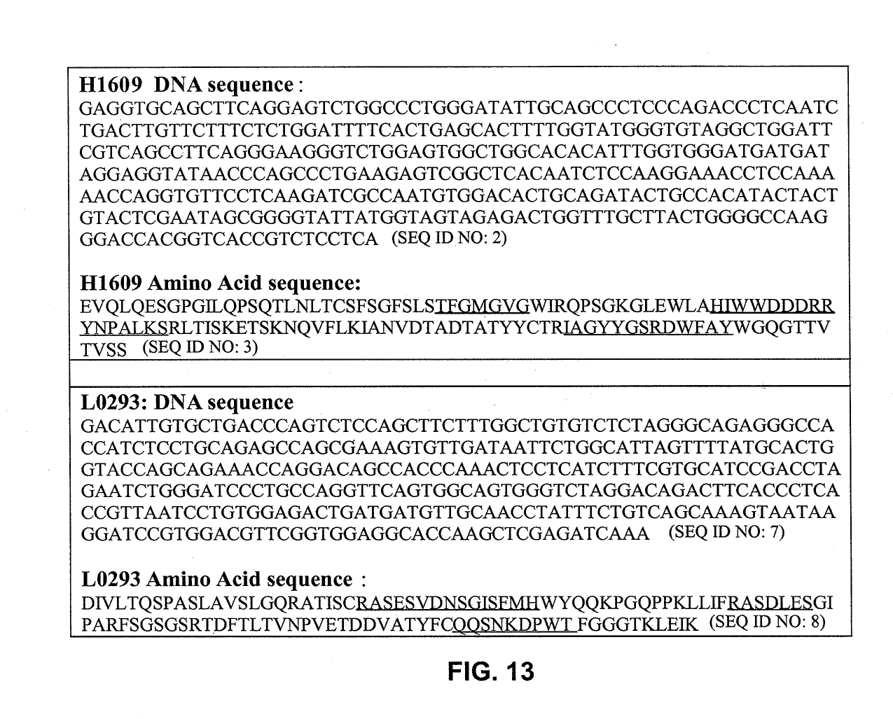

Mouse 3D2 antibody was used as a prototype for generation of a chimeric anti-CXCL13 monoclonal antibody. The variable (V) genes were isolated from a 3D2 hybridoma using standard methods. The polynucleotide and amino acid sequences of the heavy chain (H1609) and the light chain (L0293) of 3D2 are shown in FIG. 13. The VH and VK complementarity determining regions (CDRs) are underlined (SEQ ID NOs: 4, 5, 6, 9, 10, and 11, respectively).

The variable heavy (VH) gene was cloned into a mammalian expression vector that contained the human gamma 1 heavy chain gene, creating a full length chimeric heavy chain. The variable light (VK) gene was cloned into a mammalian expression vector that had the human Kappa constant gene, creating a full length chimeric light chain. In order to make the chimeric antibody, the expression vectors containing the chimeric heavy chain and the chimeric light chain were co-transfected into CHO-S cells. The monoclonal antibody (MAb) that was produced was secreted from the cells and harvested after a 3 to 6 day expression period. The resulting MAb was purified using Protein A chromatography and characterized. The resulting chimeric IgG1 antibody (“MAb 1476”) was shown to be specific for human and mouse CXCL13 by ELISA, was shown to have similar affinity on mouse and human CXCL13, and was shown to have similar functional activity as the parental mouse antibody, 3D2 (data not shown). Furthermore, MAb 1476 was able to compete with biotinylated 3D2 and 3C9 for binding on mouse and human CXCL13 in an Epitope Competition ELISA (data not shown).

Humanization of Chimeric 3D2 (MAb 1476).

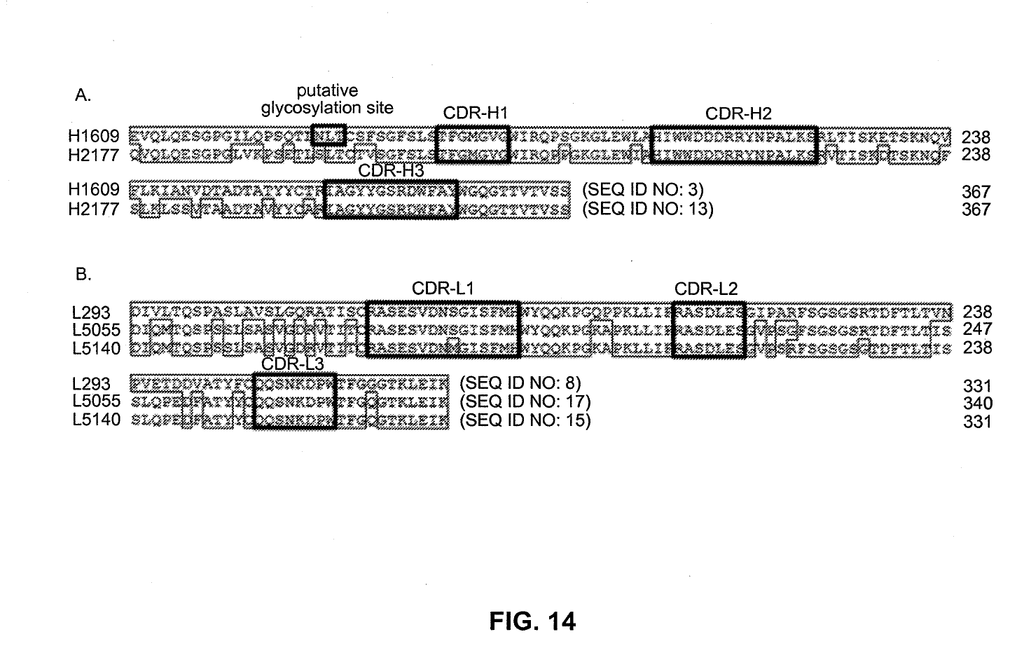

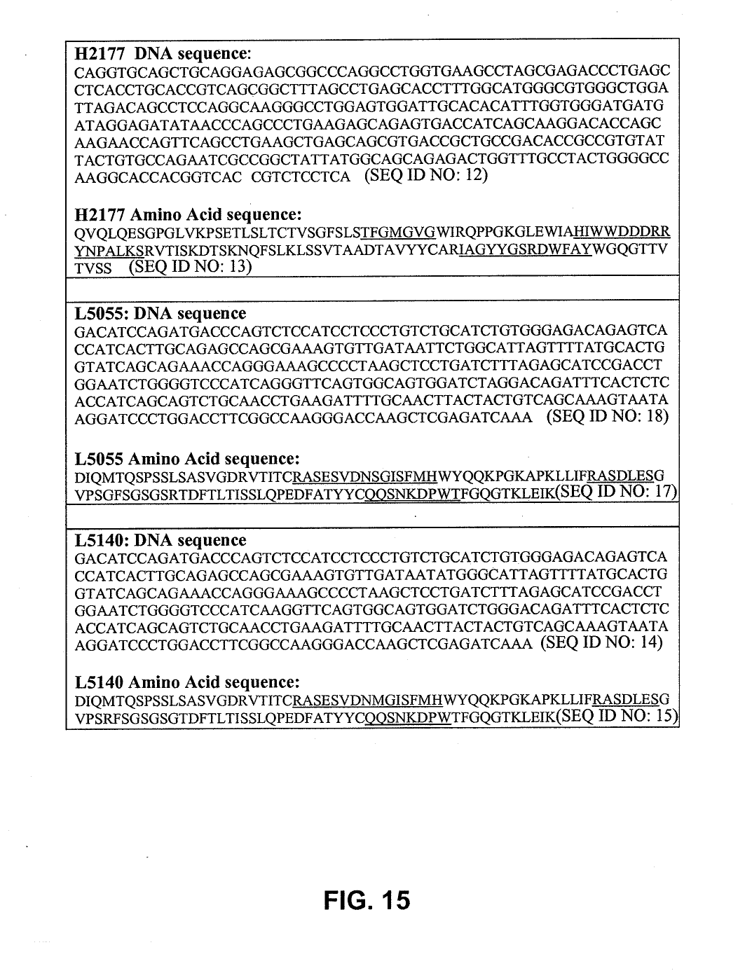

Humanization of chimeric 3D2 antibody is summarized below. The modifications that were introduced to framework regions (FWR) of heavy (H1609) and light chains (L0293) from the chimeric MAb 1476 are shown in FIGS. 14A and 14B, respectively. A putative N-linked glycosylation site (Asn-Leu-Thr) in H1609 was replaced with Ser-Leu-Thr (FIG. 14A). The mutation did not affect antibody affinity (see Table 6) and resulted in generation of “MAb 5080.” In order to improve affinity and functionality of MAb 5080, a number of variable region mutants were produced and screened by IC50 ELISA on human CXCL13. A single Serine (S) to Methionine (M) mutation at position 31 in the L5055-CDR1 as well as changes to the light chain framework region (see FIGS. 14B and 15) resulted in generation of “MAb 5261,” which demonstrated a significant improvement in affinity compared to 3D2 and MAb 5080 (Table 6). A comparison of the amino acid sequences of H1609 (SEQ ID NO: 3) and H2177 (SEQ ID NO: 13) is shown in FIG. 14A, and a comparision of L0293 (SEQ ID NO: 8), L5055 (SEQ ID NO: 17), and L5140 (SEQ ID NO: 15) is shown in FIG. 14B.

TABLE 6
Antibody affinity for recombinant human and mouse CXCL13

| | | Heavy | | Affinity (Biacore), nM | |
|---|---|---|---|---|---|
| | | Chain | Light | Human | Mouse |
| Antibody | Fc | (VH) | Chain (VK) | CXCL13 | CXCL13 |
| 3D2 | Mouse | H1609 | L0293 | 13 | 159 |
| | IgG1 | | | | |
| MAb 1476 | Human | H1609 | L0293 | 11.4 | NA |
| (chimeric 3D2) | IgG1 | | | | |
| MAb 5080 | Human | H2177 | L5055 | 14.5 | 59.2 |
| | IgG1 | | | | |
| MAb 5261 | Human | H2177 | L5140 | 5.1 | 8.1 |
| (affinity | IgG1 | | (L5055 | | |
| improved MAb | | | M31a) | | |
| 5080) | | | | | |

The polynucleotide and amino acid sequences of MAb 5080 VH and VK: H2177 (SEQ ID NO: 13) and L5055 (SEQ ID NO: 17), respectively; and MAb 5261 VH and VK: H2177 (SEQ ID NO: 12) and L5140 (SEQ ID NO: 15), respectively, are shown in FIG. 15.

MAb 5261 Specificity for CXCL13:

Similar to 3D2, specificity of MAb 5261 was assessed by specificity ELISA and Capture Epitope Competition ELISA on a panel that included recombinant chemokines (recombinant mouse, human, and cynomolgus monkey CXCL13, human IL-8/CXCL8; human IP-10/CXCL10, human MIG/CXCL9 and human SDF-1alpha/CXCL12); native human and mouse CXCL13; and various non-specific antigens (recombinant human C35, streptavidin, bovine serum albumin (BSA), human serum albumin (HAS), insulin, and hemoglobin).

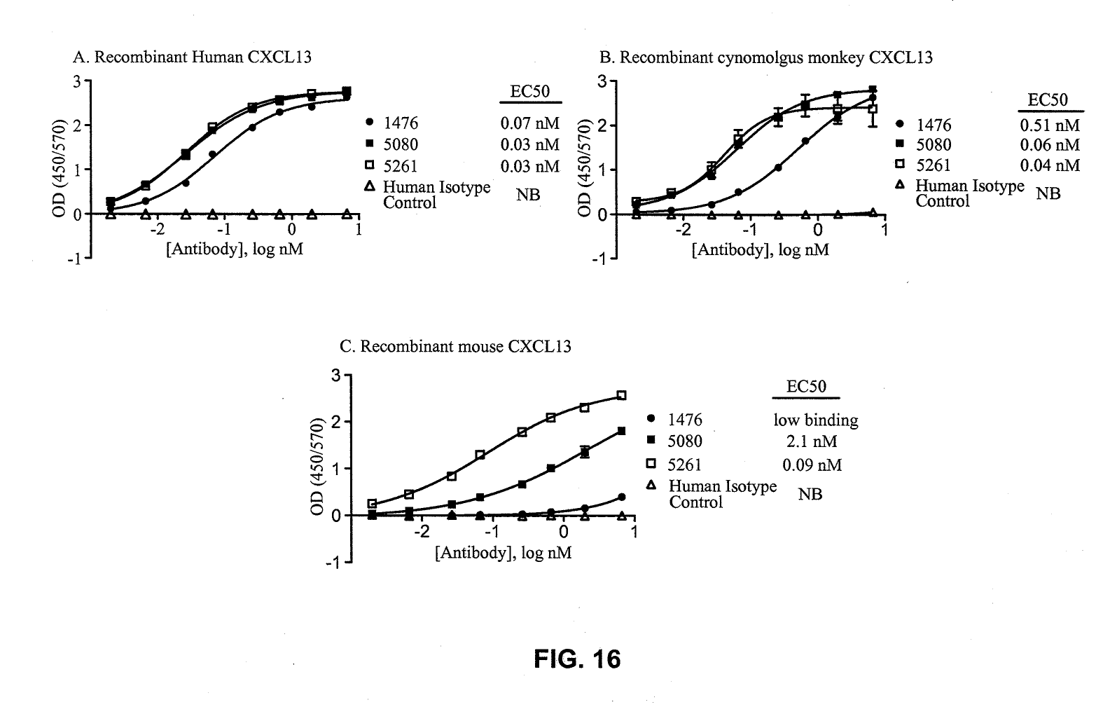

For specificity ELISA, recombinant human, cynomolgus monkey, and mouse CXCL13 were each coated at 100 nM. MAb 5261 demonstrated multi-species specificity to CXCL13 (FIGS. 16A-C). The binding of MAb 5261 on recombinant human (FIG. 16A) and cynomolgus monkey CXCL13 (FIG. 16B) was comparable to the binding of its direct “parent”, MAb 5080, and stronger than the binding of MAb 1476 (chimeric 3D2). MAb 5261 was significantly superior in binding on recombinant mouse CXCL13 compared to both MAb 1476 and MAb 5080 (FIG. 16C). The data points for each chemokine represents the mean of triplicate measurements. EC50 values were calculated from four-parameter sigmoidal curve fit (R² for the curves that produced EC50 values were 0.99).

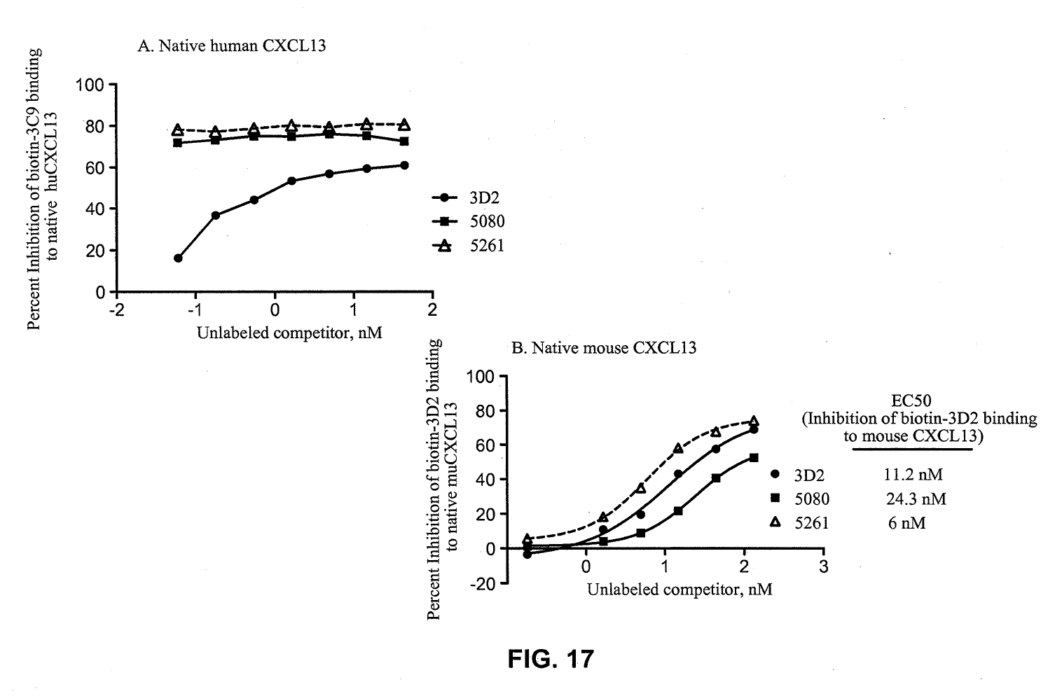

MAb 5261 binding to native human and mouse CXCL13 was determined by Capture Epitope Competition ELISA. For human ELISA, human CXCL13 (1:4 dilution of THP1 supernatant or 0.097 nM) was captured with 6.6 nM MAb 801 and detected with 0.66 nM biotin-3C9. For mouse ELISA, mouse CXCL13 (1:40 dilution of TNF-Tg organ extract) was captured with 33 nM MAb 470 and detected with 3.3 nM biotin-3D2. Each data point represents an average of duplicate measurements from one of at least three independent experiments. When tested in Capture Epitope Competition ELISA on native human (FIG. 17A) and mouse CXCL13 (FIG. 17B), MAb 5261 demonstrated superiority to both 3D2 and 5080 antibodies for binding to both human and mouse native CXCL13. Curves shown in FIGS. 17A-B were fitted using four-parameter sigmoidal curve fit (R²=0.99).

### Example 7

### Functional Characterization of Anti-CXCL13 MAb 5261

Inhibition of Human and Mouse B-Cell Migration.

The ability of MAb 5261 to inhibit human CXCL13-induced human B-cell chemotaxis was tested on both the stable cell line human pre-B-697-hCXCR5 and primary human tonsillar cells.

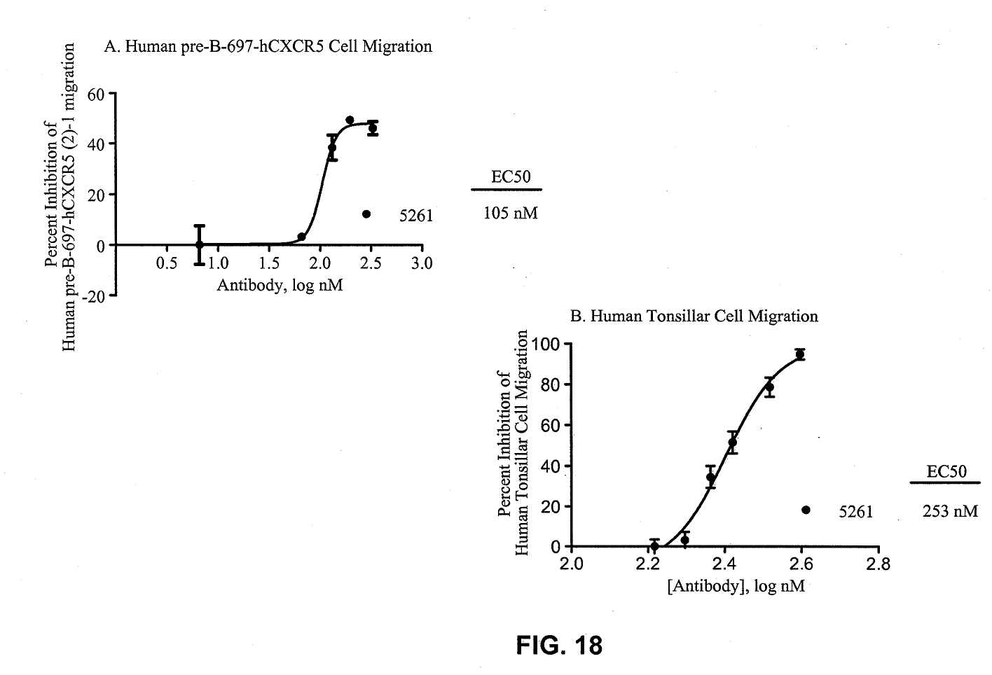

MAb 5261 inhibition of human CXCL13-induced human B-cell chemotaxis on stable cell line human pre-B-697-hCXCR5 was tested using the protocol is described in Example 2 above. The human pre-B-697-hCXCR5 cell migration inhibition by MAb 5261 is shown in FIG. 18A. For the human primary tonsillar cell studies, the tonsillar cells were obtained by the mechanical dissociation of the tissue. Cells (10⁶/upper chamber of 5-μm Transwell plate) were allowed to migrate towards 5 μg/ml human CXCL13 for 2 hours at 37° C.) Inhibition of human primary tonsillar cell migration by MAb 5261 is shown in FIG. 18B. The results shown in FIGS. 18A-B represent an average of triplicate measurements +/−SEM from one of at least three experiments. Curves were fitted using four-parameter sigmoidal curve fit (R2=0.98-0.99).

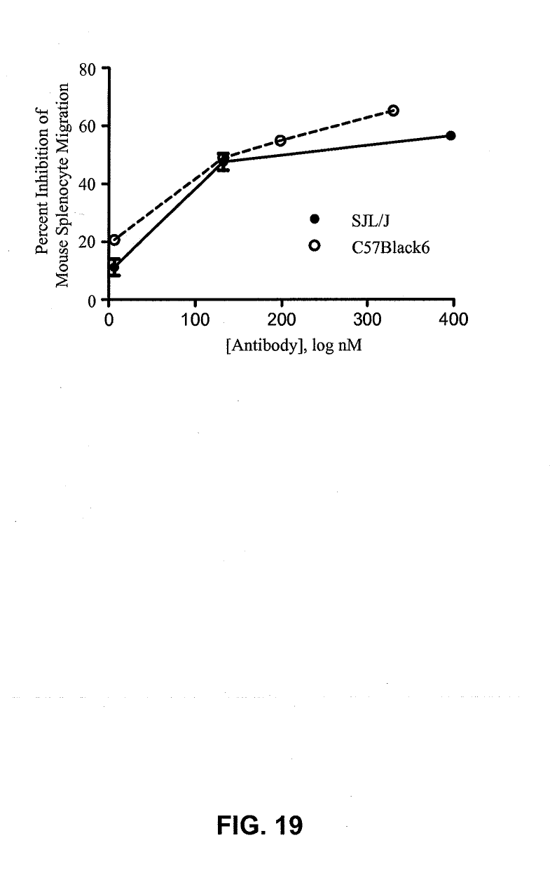

The effect of MAb 5261 on mouse CXCL13-mediated mouse B-cell chemotaxis was evaluated on mouse splenocytes from C57Black/6 and SJL/J mice as described in Example 2 above. Again migration towards human or murine SDF-1α (0.1 μg/ml) was used as a negative control to ensure CXCL13 specificity of the antibody effect (data not shown). The splenocyte migration inhibition by MAb 5261 is shown in FIG. 19 (the data from representative experiments are shown as mean of duplicate measurements +/−SD). Migration inhibition values were calculated based on the values obtained with corresponding Isotype controls.

As shown in FIGS. 18 and 19, MAb 5261 inhibited both human and mouse CXCL13-dependent chemotaxis. The differences in EC50 values between cultured and primary cells (see FIG. 18) could likely be attributed to differences in human CXCL13 concentrations, e.g., 97 nM (1 μg/ml) was used in human pre-B-697-hCXCR5 cell migration and 485 nM (5 μg/ml) was used in human tonsillar cell migration.

Inhibition of Human CXCL13-Mediated Endocytosis of Human CXCR5 Receptor.

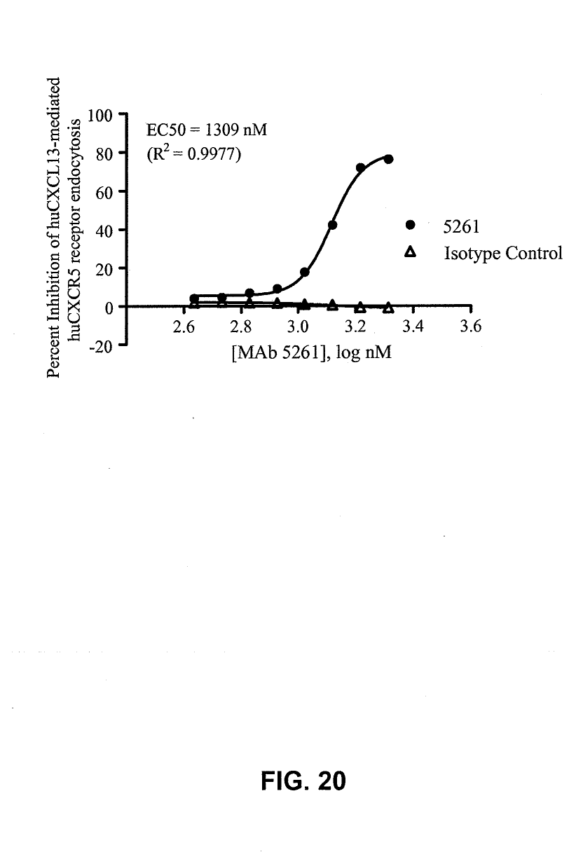

This experiment was done using human pre-B-697-hCXCR5 cells treated with human CXCL13 to induce endocytosis of human CXCR5 receptor according to the methods described above in Example 3. The amount of human CXCL13 was 2 μM, which was higher than the amount used in the 3D2 endocytosis assay (i.e., 0.485 μM) shown in Example 3, thus differences in EC50 values were observed Inhibition of CXCR5 receptor endocytosis by MAb 5261 is shown in FIG. 20. The results are shown as an average of triplicate measurements from one of at least three independent experiments. The curve was fitted using four-parameter sigmoidal curve fit (R2=0.99).

### Example 8

### Generation and Characterization of a Murine Version of Anti-CXCL13 MAb 5261

MAb 5261 contains human heavy and light variable regions and human IgGamma1-F allotype as well as human kappa. A murine counterpart (“MAb 5378”) was engineered with mouse IgG2a (Gamma 2a chain). IgG2a isotype has close similarities to human IgG1, including the ability to fix complement and bind to Fc receptor. MAb 5378 contains the same human heavy and light chain variable genes as MAb 5261 along with mouse IgG2a constant and mouse kappa, respectively.

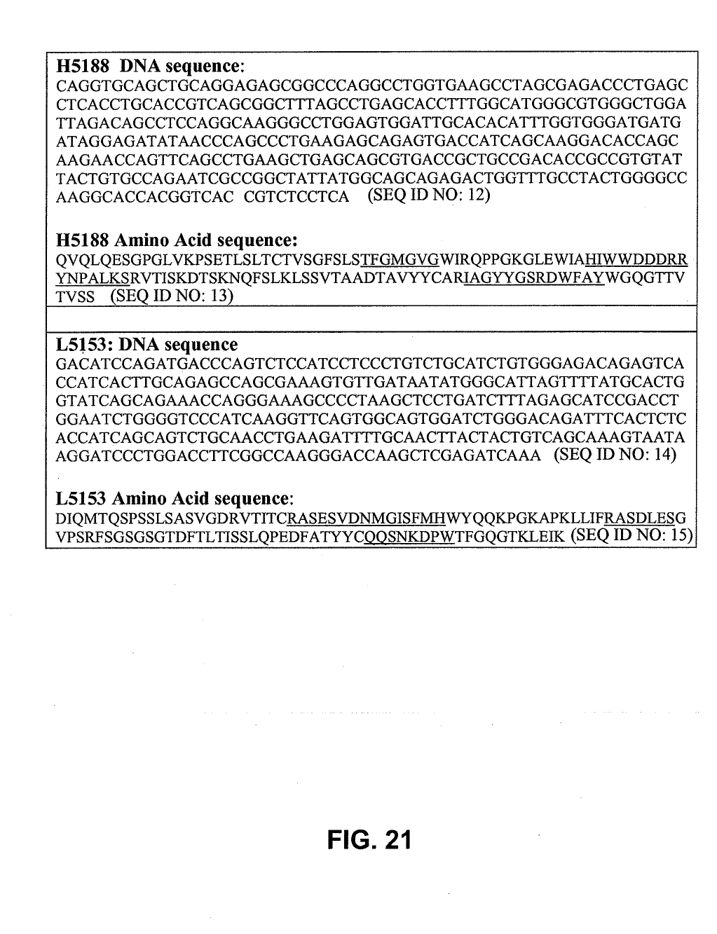

A common restriction site among the heavy and light chain expression plasmids was used to allow for the changing of isotypes. Generation of the isotype species was achieved through restriction digestion, ligation, and transformation. Specifically, for the MAb 5261 heavy chain, the variable region of the gene was digested with restriction endonucleases and ligated into comparable sites in an expression plasmid that contained the mouse IgG2a constant region in order to make the heavy chain for MAb 5378 (H5188). Similarly, for the MAb 5261 light chain, the variable region of the gene was digested with restriction endonucleases and ligated into comparable sites in an expression plasmid that contains the mouse IgKappa constant region to make the light chain for MAb 5378 (L5153). The polypeptide and amino acid sequences of the variable regions of MAb 5378 light and heavy chains are identical to MAb 5261 and are shown in FIG. 21. The VH and VK complementarity determining regions (CDRs) are underlined (SEQ ID NOs: 4, 5, 6, 16, 10, and 11, respectively).

MAb 5378 Affinity Measurements.

Affinity measurments of MAb 5378 for recombinant human and mouse CXCL13 were measured by BIACORE® using methods similar to those described in Example 1. Mab 5378 affinity for recombinant human and mouse CXCL13 was compared to MAb 5261 and 3D2. As shown in Table 7, the affinity measurements (nM) of MAb 5261 and MAb 5378 for both human and mouse chemokines were significantly improved compared to the 3D2.

TABLE 7
Affinities of 5261, 5378, and 3D2 for recombinant human and
mouse CXCL13

| | | Affinity, nM | |
|---|---|---|---|
| Antibody | Fc | Human CXCL13 | Mouse CXCL13 |
| 5261 | Human IgG1 | 5.1 | 8.1 |
| 5378 | Mouse IgG2a | 4.5 | 4.2 |
| 3D2 | Mouse IgG1 | 13 | 159 |

MAb 5378 Epitope Mapping.

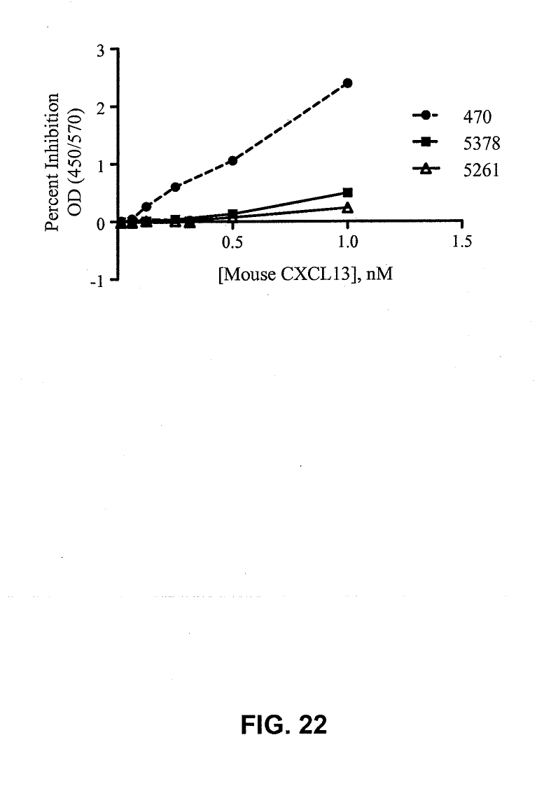

An Epitope Competition ELISA experiment was performed to determine if MAb 5378 shared a binding epitope on mouse CXCL13 with MAb 5261. Recombinant mouse CXCL13 was captured on the plate with 1 μg/ml of MAb 470, 5378 or 5261 (control). Antibody/chemokine interactions were detected with 0.5 μg/ml (3.3 nM) of biotinylated MAb 5261 followed by Streptavidin-HRP. Commercial rat anti-mouse antibody MAb 470 was also included into the study. The ability of mouse CXCL13, pre-incubated with either MAbs 470 or 5378, to bind biotinylated MAb 5261 was evaluated using Epitope Competition ELISA. It was shown (FIG. 22), that MAb 5378 shared a mouse CXCL13 binding epitope with MAb 5261, but not with MAb 470. Thus, MAb 5378 was shown to retain epitope binding and affinity, which was needed in order for MAb 5378 to be used as a surrogate for MAb 5261 in animal model studies. Furthermore, the epitope binding results described throughout the Examples can be summarized as showing that 3D2, 3C9, MAb 1476, MAb 5080, MAb 5261, and MAb 5378 all bind the same epitope of human CXCL13.

MAb 5378 Specificity for CXCL13.

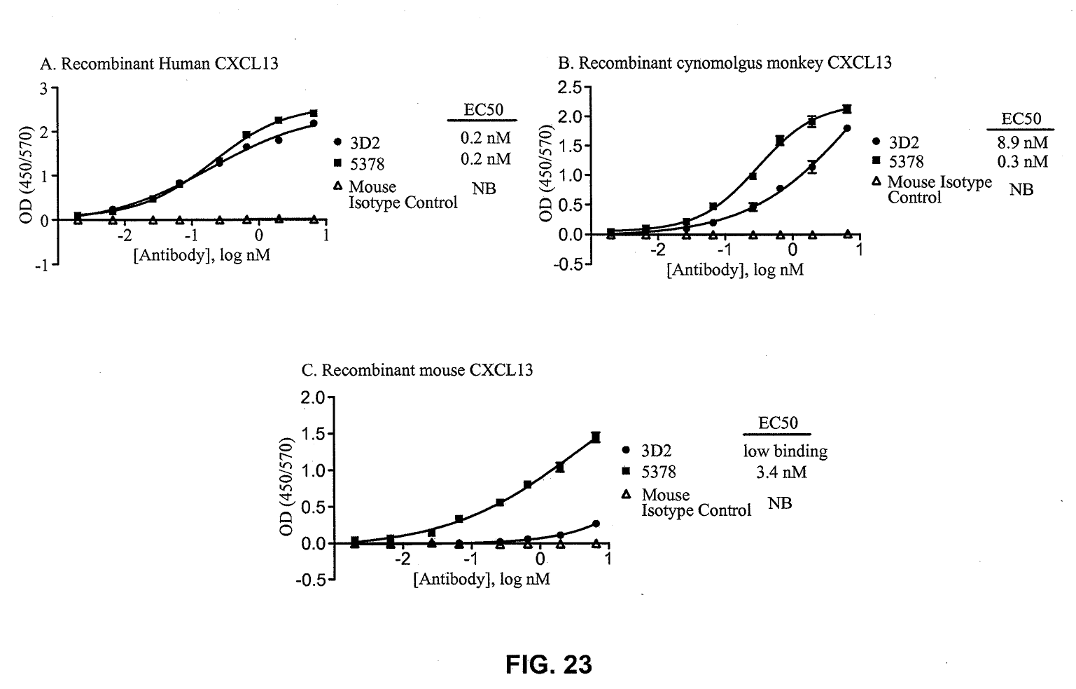

Specificity of MAb 5378 was evaluated on recombinant human, mouse and cynomolgus monkey CXCL13 (FIG. 23) and a panel of recombinant chemokines and various antigens (recombinant chemokines (mouse, human and cynomolgus monkey CXCL13, human IL-8/CXCL8, human IP-10/CXCL10, human MIG/CXCL9, and human SDF-1alpha/CXCL12); native human and mouse CCL13; and various non-specific antigens (recombinant human C35, streptavidin, bovine serum albumin (BSA), human serum albumin (HAS), insulin, hemoglobin) (data not shown). Specificity ELISA was performed as described in Example 1. In particular, each chemokine was coated at 100 nM. As shown in FIG. 23, MAb 5378 was compared to mouse antibody 3D2 and control (mouse IgG). MAb 5378 was superior to 3D2, to varying degree, in binding to the chemokines from all three species. The most significant differences in binding were observed on mouse CXCL13 showing the potential advantage of MAb 5378 over 3D2 in animal studies. Each data point represents mean of triplicate measurements. EC50 values were calculated from four-parameter sigmoidal curve fit (R² for the curves that produced EC50 values were 0.99).

Inhibition of Human and Mouse B-Cell Migration was Tested for MAb 5378.

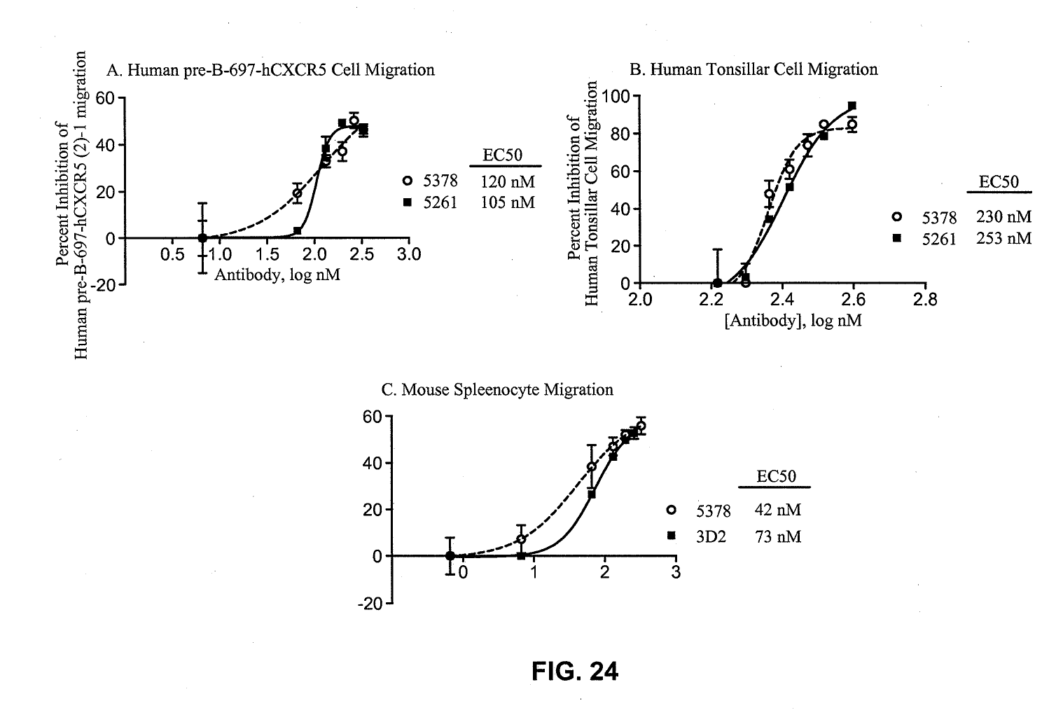

Ability of MAb 5378 to interfere with CXCL13-dependent chemotaxis of mouse and human B-cells was tested in migration assays involving cultured (human pre-B-697-hCXCR5 cells; FIG. 24A) and primary (human tonsillar cells; FIG. 24B) human cells as well as mouse spleenocytes (FIG. 24C) using the methods described in Examples 2, 7, and 2 respectively. The chemokine concentrations were: 97 nM of huCXCL13 for human pre-B-697-huCXCR5 migration; 485 nM of huCXCL13 for human tonsillar cell migration; and 500 nM of muCXCL13 for mouse spleenocyte migration. Migration towards human or murine SDF-1alpha was used as a negative control (not shown). Migration inhibition values were calculated based on the values obtained with corresponding Isotype Controls. The results shown in FIGS. 24A-C represent an average of triplicate measurements +/−SEM from one of at least three experiments. Curves were fitted using four-parameter sigmoidal curve fit (R²=0.99). In the human migration assays, MAb 5378 was compared to MAb 5261, and in the mouse migration assays, MAb 5378 was compared to 3D2. In both cases, MAb 5378 successfully inhibited CXCL13-induced human and mouse cell migration to a degree comparable to MAb 5261 and slightly superior to 3D2.

Inhibition of Human CXCL13-Mediated Endocytosis of Human CXCR5 Receptor.

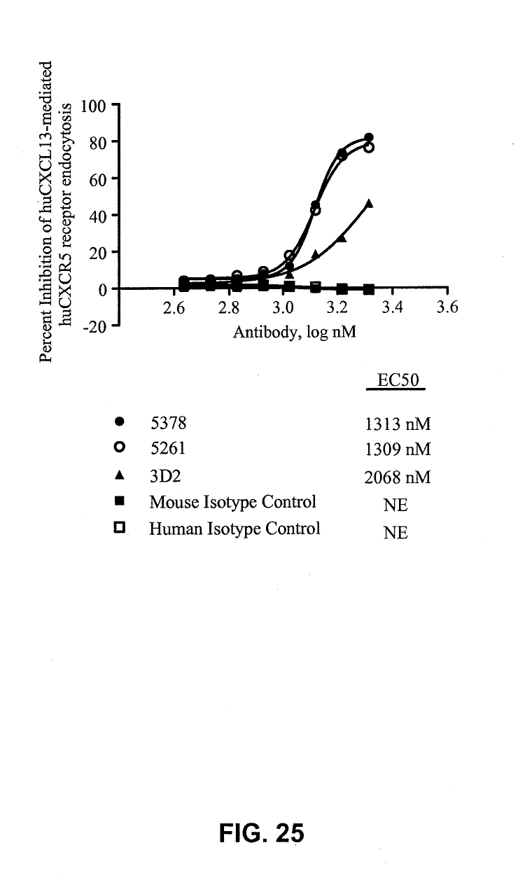

MAb 5378 was compared to its prototype MAb 5261 and mouse anti-human CXCL13 antibody 3D2 in a human CXCL13-mediated human CXCR5 receptor internalization assay using the methods described in Example 3. As shown in FIG. 25, MAb 5378 was identical to MAb 5261 and significantly superior to 3D2 in its ability to inhibit human CXCR5 receptor internalization. The data points for MAb 5261 and MAb 5378 represent average of measurements from two independent experiments. Data points for 3D2 and Isotype Controls represent average of triplicate measurements from a single experiment. Curve was fitted using four-parameter sigmoidal curve fit (R²=0.99).

### Example 9

### Evaluation of Anti-CXCL13 Antibodies in Mouse Disease Model for Rheumatoid Arthritis

Murine Model of Rheumatoid Arthritis.

Collagen-induced arthritis (CIA) in mice and rats is a well-established model of human Rheumatoid arthritis (RA). The disease is typically induced by intradermal injection of bovine type II collagen emulsified in Complete Freund's Adjuvant (CFA) and is characterized by production of mouse collagen antibodies and, subsequently, progressive development of arthritis in the paws.

CIA-1: Anti-Arthritic Efficacy of MAb 5378 in CIA Model Using DBA1/J Mice.

The disease was induced in DBA1/J mice by subcutaneous immunization with 100 μg of bovine type II collagen in CFA enhanced with 100 μg of heat-killed M. tuberculosis H37Ra, followed by boost immunization on Day 21 with 100 μg of bovine type II collagen in Incomplete Freunds' Adjuvant (IFA). The animals were scored for macroscopic signs of arthritis (see Table 8) three times weekly and the Arthritic Index (AI) was calculated by addition of individual paw scores (the maximum arthritic index that could be achieved in any given animal was 16).

TABLE 8
Macroscopicsigns of CIA in mice

| Arthritic Score | Description |
|---|---|
| 0 | No visible effects of arthritis |
| 1 | Edema and/or erythema of 1 digit |
| 2 | Edema and/or erythema of 2 digits |
| 3 | Edema and/or erythema of more than 2 |
| | digits |
| 4 | Severe arthritis of entire paw and digits |

Prophylactic treatment started one day before boost immunization, i.e., on Day 20 post-induction in animals with low AI of 2-6, and consisted of the following treatment groups (10 mice per group):
A. Mouse IgG Isotype (control)
B. MAb 5378
C. etenercept (TNF inhibitor; positive control)

The mice had been given either intraperitoneal (Mouse IgG and MAb 5378) or subcutaneous (etenercept) injections of 0.6 mg (30 mg/kg) of antibody twice a week for three weeks. The study was terminated on Day 41 postinduction.

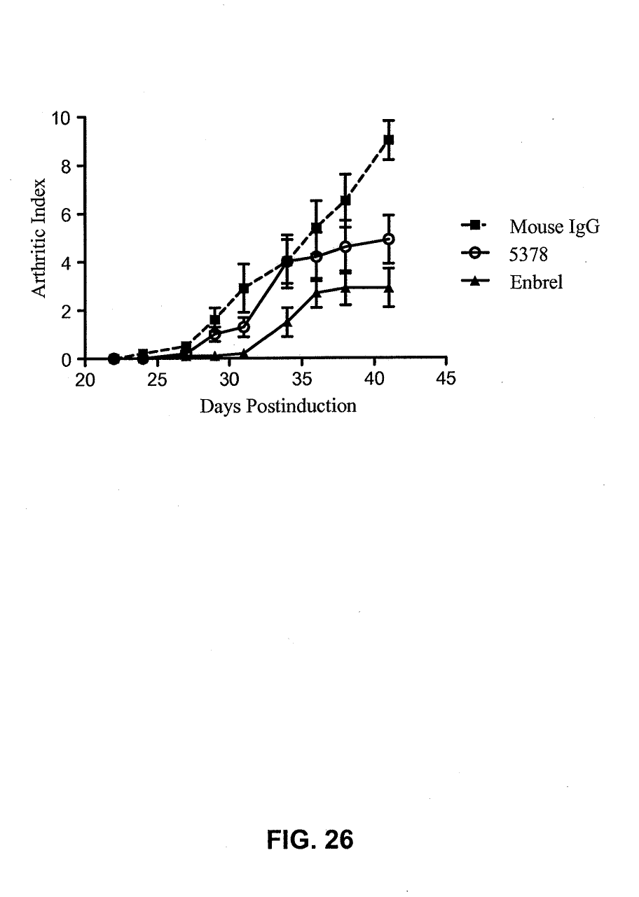

As shown in FIG. 26 prophylactic treatment with MAb 5378 resulted in a decreased rate of disease development and significant inhibition of disease severity, which became evident at the study endpoint. Statistically significant differences were observed at the study endpoint (Day 41) between Mouse IgG and MAb 5378 treated groups (P<0.05) and Mouse IgG and etenercept treated groups (P<0.05). The inhibitory effect of MAb 5378 was not statistically different from the inhibitory effect of positive control agent etenercept (P>0.05).

CIA-2: Anti-Arthritic Efficacy of MAb 5378 in CIA Model in DBA1/J Mice.

A second CIA study with MAb 5378, “CIA-2,” was performed. In this study, the disease was induced in DBA1/J mice as described above for CIA-1. Again prophylactic treatment started one day before boost immunization, on Day 20 postinductions in animals with low AI of 2-6. In addition to etenercept, commercial rat anti-murine CXCL13 antibody MAb 470 was used as a control. The study therefore included the following groups:
A. Mouse IgG Isotype control
B. MAb 5378
C. etenercept (TNF inhibitor; positive control)
D. MAb 470

The mice were given either intraperitoneal (Mouse IgG, MAb 470 and MAb 5378) or subcutaneous (etenercept) injections of 0.6 mg (30 mg/kg) of antibody twice a week for three weeks. The study was terminated on Day 42 postinduction.

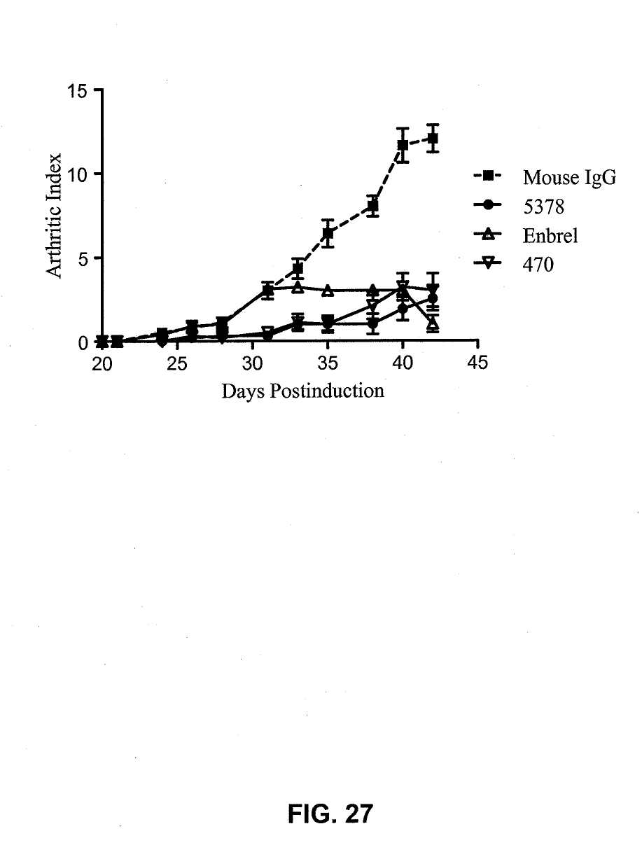

As evident from FIG. 27, prophylactic treatment with MAb 5378 again resulted in a decreased rate of disease development and significant inhibition of disease severity throughout the study as well as at the endpoint (Day 42). Statistically significant differences were observed between Mouse IgG and MAb 5378 treated groups (P<0.05) and Mouse IgG and MAb 470 treated groups (P<0.05). The inhibitory effect of MAb 5378 was not statistically different from the inhibitory effects of positive control agent etenercept and rat anti-murine CXCL13 antibody MAb 470 (P>0.05).

GC-1: Effect of MAb 5378 on Germinal Center Formation in Immunized BALB/c Mice.

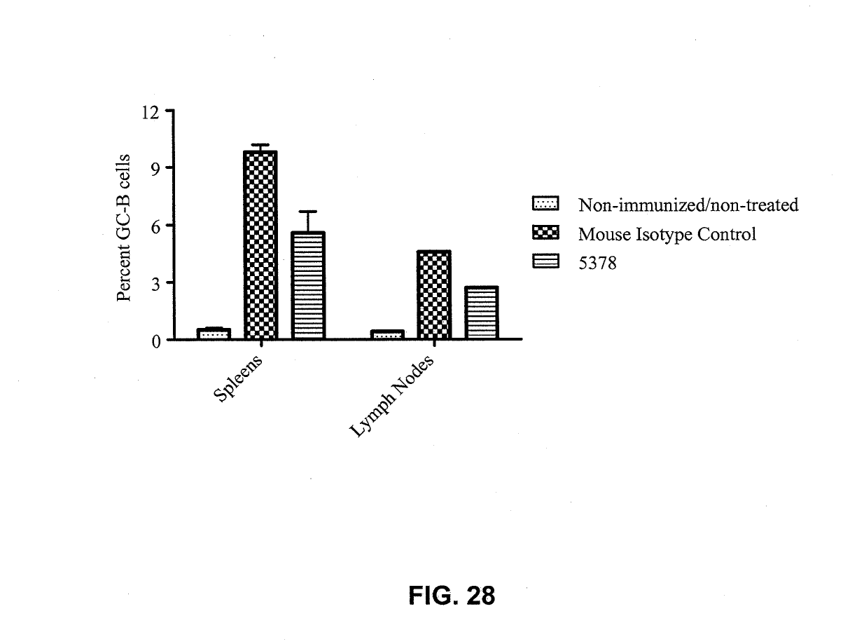

Given the successful performance of our anti-CXCL13 antibodies in animal models of autoimmunity, the possible mechanism of action involving disruption of ectopic germinal center formation was tested. Germinal centers in MAb 5378-treated BALB/C mice immunized with 100 μg 4-hydroxy-3-nitrophenylacetyl-chicken-g-globulin (NP-CGG) precipitated in 100 ul of alum were examined. The animals were injected intraperitoneally with total of 0.6 mg (30 mg/kg) a week of either Mouse isotype control (0.6-mg weekly injections) or MAb 5378 (0.3-mg bi-weekly injections). The injections started one week before and continued through one week after NP-GCC immunization. Germinal center formation was evaluated on day 10 post-challenge. Single cell suspensions from spleens and lymph nodes were analyzed by flow cytometry for the presence of various B-cell (activated GC B-cells; follicular and marginal zone B-cells) and T-cell (CD4+ and CD8+) subsets. Although MAb 5378 produced no effect on T-cells or follicular and marginal zone B-cells (data not shown), germinal center B-cells (B220+/CD38low/PNA+) were reduced in spleens and lymph nodes by 43% and 41%, respectively (FIG. 28). Spleen group means were compared by using unpaired student t-test. The reduction in GC-B cells was statistically significant in MAb 5378-treated spleens compared to Mouse IgG treated group (P<0.05). The cells recovered from lymph nodes were very low in numbers and therefore were pooled (as a result, no statistical analysis was performed with the data from the lymph nodes).

### Example 10

### Evaluation of Anti-CXCL13 Antibodies in Mouse Model for Helicobacter Infection

Murine Model of Helicobacter Infection.

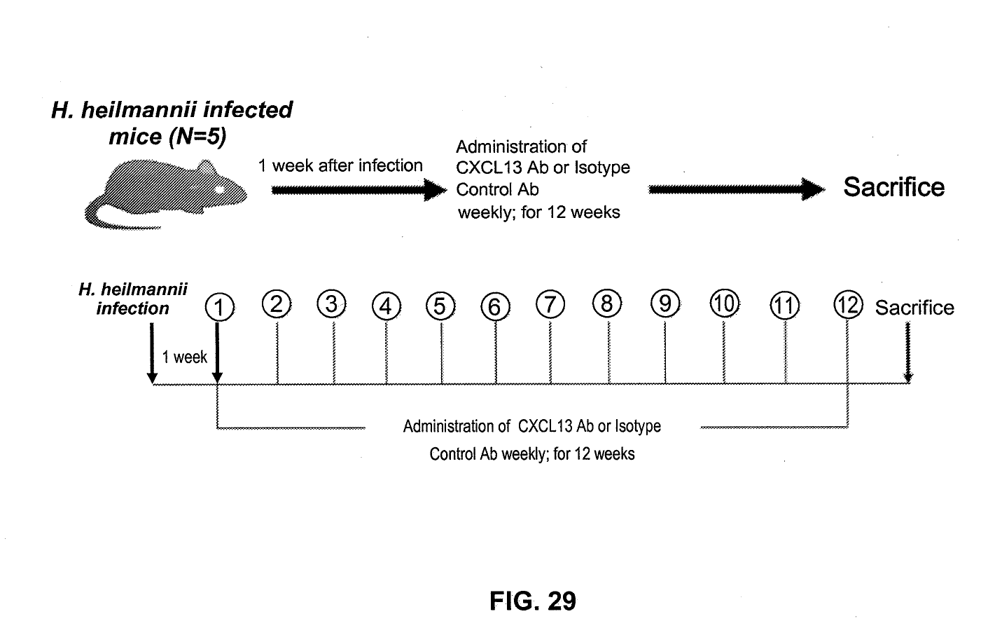

Heliobacter species such as H. heilmannii and H. Pylori induce gastric MALT lymphoma in patients. A mouse model of Heliobacter induced gastric lymphoid follicles was described in Nobutani et al., FEMS Immunol Med Microbiol 60:156-164 (2010), which is incorporated herein by reference in its entirety. The Nobutani et al. mouse model was used herein to test the effect of anti-CXCL13 antibody in reducing gastric lymphoid follicles. The treatment schedule for H. heilmannii infection of mice and antibody administration used in this Example is shown in FIG. 29.

In particular, C57BL/6J mice were infected with H. heilmannii. Starting one week post-infection, the mice were administered either Isotype antibody control or anti-CXCL13 antibody (MAb 5378) weekly for twelve weeks. Anti-CXCL13 MAb 5378 (a mouse IgG2a isotype) was formulated in PBS, pH 7.2 at 3 mg/ml. Mice were injected with 200 microliters (600 micrograms) intraperitoneally starting day 7 post-infection and weekly thereafter. The Isotype control was an independent monoclonal mouse IgG2a anibody.

Gastric samples from the mice were evaluated by PCR for expression of H. heilmannii specific 16s rRNA genes to confirm infection. PCR amplification reactions involved 1× reaction buffer [20 mM Tris/HCl(pH8.0), 100 mM KCl, 0.1 mM EDTA, 1 mM DTT, 0.5% Tween-20, 0.5% Nonidet P40, and 50% glycerol] containing 1 unit of Taq DNA polymerase (TOYOBO, Osaka, Japan); 10 nmols of each deoxynucleotide triphosphate; 10 μmols of each oligonucleotide primer; and 1 μl of the diluted DNA, which was prepared by 1:10 dilution of the original samples with a DNA concentration of approximately 20-100 ng/μl, in a final volume of 50 μl. Cycling conditions for the 16s rRNA reactions involved 35 cycles of 94° C. for 30 seconds, 56° C. for 30 seconds, and 72° C. for 30 seconds.

The H. heilmannii specific 16s rRNA gene PCR primers are shown below:

| | |
|---|---|
| F: 5′-TTGGGAGGCTTTGTCTTTCCA-3′ | (SEQ ID NO: 24) |
| R: 5′-GATTAGCTCTGCCTCGCGGCT-3′ | (SEQ ID NO: 25) |

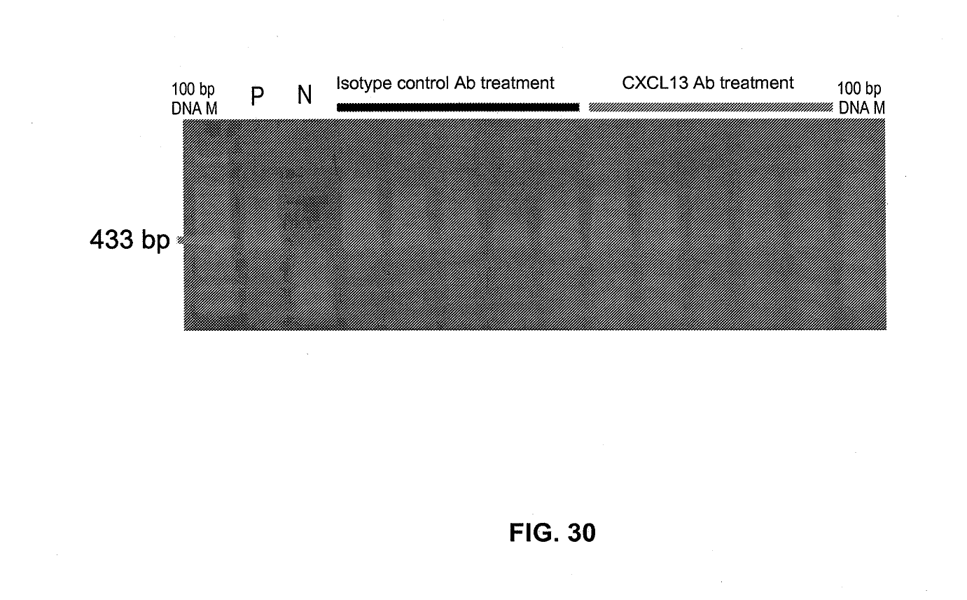

The results for expression of H. heilmannii specific 16s rRNA genes amplified in all gastric samples obtained from H. heilmannii infected mice are shown in FIG. 30. These results show that all of the treated mice were positive for H. heilmannii infection.

CXCL13 Expression in Gastric Mucosa of Helicobacter Infected Mice.

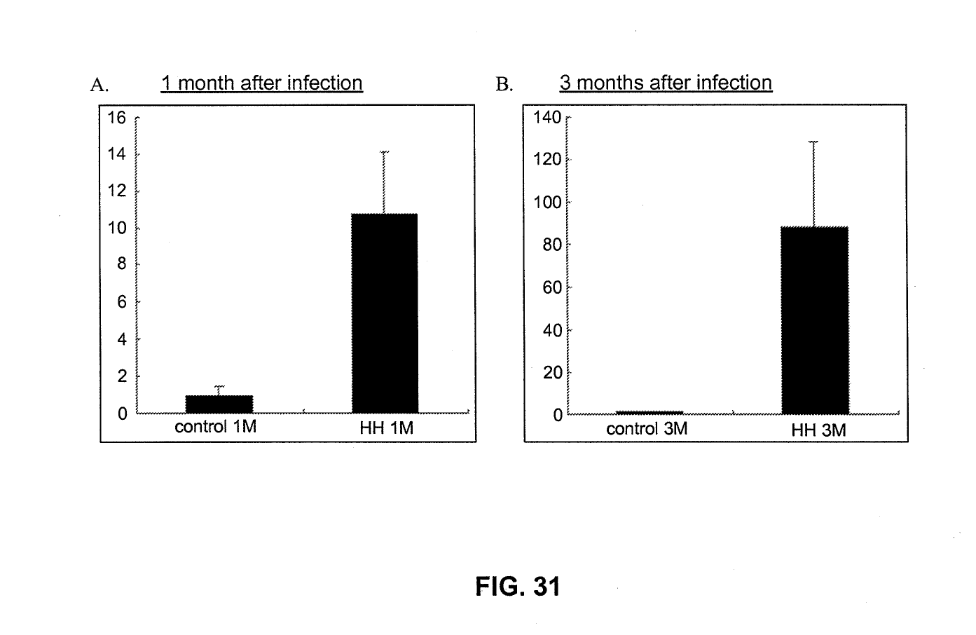

The mRNA expression levels of CXCL13 in the gastric mucosa of H. heilmannii infected and noninfected mice was determined by real-time quantitative PCR. The mRNA expression of CXCL13 in the gastric mucosa of H. heilmannii infected mice compared to noninfected wild-type control mice one month and three months after infection is shown in FIGS. 31A and 31B, respectively. These results show an increase in CXCL13 expression in H. heilmannii infected mice.

CXCL13 Expression in Gastric Mucosa of Antibody Treated Helicobacter Infected Mice.

The mRNA expression levels of CXCL13 in the gastric mucosa of H. heilmannii infected mice after treatment with Isotype control or anti-CXCL13 antibody was determined by reverse transcripiton PCR. The mucosal and submucosal layers of the stomach were removed from the muscularis and serosa, and then homogenized with 1 ml of TRIZOL Regent (Invitrogen). RNA was extracted from the homogenates according to the manufacturer's instructions. RNA was subjected to the reverse transcription reaction using a High Capacity cDNA Reverse Transcription Kit (Applied Biosystems, Foster City, Calif.) according to the manufacturer's protocol. PCR amplification reactions involved 1× reaction buffer [20 mM Tris/HCl (pH8.0), 100 mM KCl, 0.1 mM EDTA, 1 mM DTT, 0.5% Tween-20, 0.5% Nonidet P40, and 50% glycerol] containing 1 unit of Tag DNA polymerase (TOYOBO, Osaka, Japan); 10 nmols of each deoxynucleotide triphosphate 10 pmols of each oligonucleotide primer; and 1 μl of the diluted DNA, which was prepared by 1:10 dilution of the original samples with a DNA concentration of approximately 100 ng/μl, in a final volume of 50 μl. Cycling conditions for the CXCL13 and β-actin reactions involved 94° C. for 2 min, 35 cycles of 94° C. for 30 seconds, 55° C. for 30 seconds, and 72° C. for 1 min.

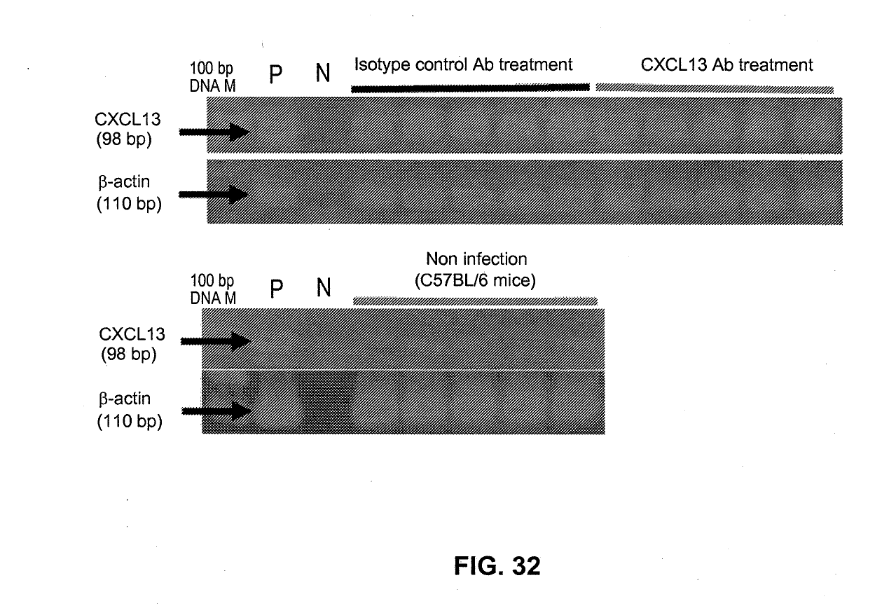

FIG. 32 shows expression of CXCL13 and β-actin control mRNA in the stomach of H. heilmannii infected mice after Isotype control or anti-CXCL13 antibody treatment. These results show that CXCL13 was not expressed in noninfected wild-type mice, but was expressed in all H. heilmannii infected mice.

Anti-CXCL13 Antibody Treatment Reduces Gastric Lymphoid Follicles in Helicobacter Infected Mice.

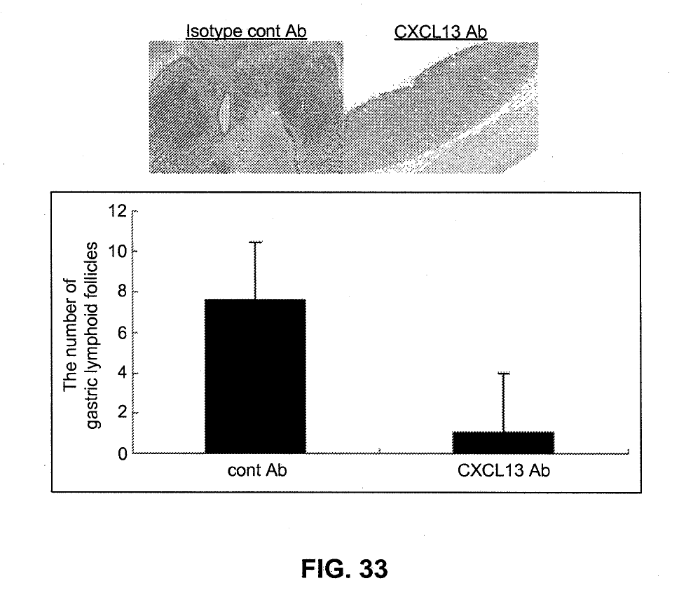

The stomachs of mice three months after H. heilmannii infection were resected and opened at the greater curvature. Half of the stomach was embedded in paraffin wax, and the paraffin-embedded tissues were sliced and stained with hematoxylin and eosin (H&E). FIG. 33 shows H&E stained images of stomach from the Isotype control (left panel) and anti-CXCL13 antibody (right panel) treated mice. The number of gastric lymphoid follicles were counted for Isotype control and anti-CXCL13 antibody samples. The results are depicted in the lower panel of FIG. 33. These results show a reduction in gastric follicles in H. heilmannii infected mice treated with anti-CXCL13 antibody relative to control treatment.

### Example 11

### Method of Epitope Mapping

Epitope mapping was performed to identify the linear or non-linear, discontinuous amino acid sequence within an antigenic protein that is recognized by a particular monoclonal antibody. A general approach for epitope mapping requires the expression of the full-length protein, as well as various fragments (i.e., truncated forms, or peptides) of the protein, generally in a heterologous expression system. These various recombinant proteins are then used to determine if the specific monoclonal antibody is capable of binding one or more of the truncated forms of the target protein. Through the use of reiterative truncation and the generation of recombinant peptides with overlapping amino acid regions, it is possible to identify the region that is recognized by the monoclonal antibody under investigation. Western blot analysis, ELISA, or immunoprecipitation can be employed to determine if the specific monoclonal antibody under investigation is capable of binding one or more of the recombinant protein fragments. This approach can ultimately identify the peptide regions that contains the epitope and, in some cases, to refine the epitope precisely to an approximately 5-15 amino acid sequence. An epitope can be a continuous linear sequence approximately 5-15 amino acids in length, nonlinear (e.g., discontinuous with the antibody binding to a site on the protein composed of different sections of the peptide chain), or both linear and nonlinear.

Systematic techniques for identifying epitopes are known in the art, and one general approach requires expression of the full length protein as well as various fragments (i.e., truncated forms, or peptides) of the protein, generally in a heterologous expression system (e.g., RTS System, “Rapid Translation System” Roche Applied Science). The recombinant proteins, fused with an N-terminal protein (e.g., GFP), are then used to determine if the specific monoclonal antibody is capable of binding one or more of the truncated forms of the protein. Through the use of reiterative truncation and generation of recombinant proteins with overlapping amino acid regions, and by Western blot, ELISA, and/or immunoprecipitation methods, it is possible to identify the region that is recognized by the monoclonal antibody under investigation.

### Example 12

### Characterization of the Epitopes of VX5/5261 Antibodies

Linear Epitope mapping for anti-CXCL13 antibody VX5/5261 was carried out essentially via the iterative process described above. In this project, the determination of peptide-antibody binding was performed by incubation of serum samples with a ProArray Ultra™ peptide microarray. This peptide array was generated as 15-mer peptides overlapping by 10 amino acids from the human CXCL13 primary sequence. The CXCL13 array contained sixteen unique peptides. The peptide array(s) was incubated with VX15/5261 and a fluorescently labelled secondary antibody, which binds selectively to an antigen-specific antibody (e.g. anti-human) was then added. After several washing steps the ProArray Ultra™ arrays were dried and scanned using a high-resolution fluorescence microarray scanning system.

Initial studies using the iterative process described above identified the epitope of the monoclonal antibody designated as VX15/5261 as YTSLRCRCVQESSVF (SEQ ID NO: 26, corresponding to residues 28 through 42 of the full-length human CXCL13 amino acid sequence set forth in SEQ ID NO: 19).

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

That which is claimed:

1. An isolated antibody or antigen-binding fragment thereof that specifically binds to CXCL13, wherein said antibody or antigen-binding fragment thereof is selected from the group consisting of:
(a) a monoclonal antibody that binds to an epitope within the sequence set forth in SEQ ID NO: 26;
(b) a monoclonal antibody that binds to an epitope comprising an amino acid sequence having at least 40% identity to SEQ ID NO: 26;
(c) a monoclonal antibody that binds to an epitope comprising an amino acid sequence identical to SEQ ID NO: 26, except for 9 or fewer amino acid substitutions; and
(d) an antigen-binding fragment of any one of (a)-(c).

2. An isolated antibody or antigen-binding fragment thereof that specifically binds to CXCL13, wherein said antibody or antigen-binding fragment thereof binds to the same CXCL13 epitope as a reference monoclonal antibody or antigen-binding fragment thereof selected from the group consisting of:
(a) the monoclonal antibody 5261;
(b) the monoclonal antibody 5378;
(c) the monoclonal antibody 5080;
(d) the monoclonal antibody 1476;
(e) the monoclonal antibody 3D2;
(f) a monoclonal antibody comprising a heavy chain variable region (VH) and a light chain variable region (VL) comprising the amino acid sequence set forth in SEQ ID NO: 13 and SEQ ID NO: 15, respectively;
(g) a monoclonal antibody comprising a VH and VL comprising the amino acid sequence set forth in SEQ ID NO: 13 and SEQ ID NO: 17, respectively;
(h) a monoclonal antibody comprising a VH and VL comprising the amino acid sequence set forth in SEQ ID NO: 3 and SEQ ID NO: 8, respectively;
(i) a monoclonal antibody comprising VH domain comprising a Chothia-Kabat heavy chain complementarity determining region-1 (VH-CDR1) amino acid sequence set forth in SEQ ID NO: 4; a Kabat heavy chain complementarity determining region-2 (VH-CDR2) amino acid sequence set forth in SEQ ID NO: 5; and a Kabat heavy chain complementarity determining region-3 (VH-CDR3) amino acid sequence set forth in SEQ ID NO: 6; and a VL domain comprising a Kabat light chain complementarity determining region-1 (VL-CDR1) amino acid sequence set forth in SEQ ID NO: 16 or 9; a Kabat light chain complementarity determining region-2 (VL-CDR2) amino acid sequence set forth in SEQ ID NO: 10; and a Kabat light chain complementarity determining region-3 (VL-CDR3) amino acid sequence set forth in SEQ ID NO: 11; and
(j) an antigen-binding fragment of any one of (a)-(i).

3. An isolated antibody or antigen-binding fragment thereof that specifically binds to CXCL13, wherein said binding molecule competitively inhibits a reference monoclonal antibody or antigen-binding fragment thereof from specifically binding to CXCL13, wherein said monoclonal antibody or antigen-binding fragment thereof is selected from the group consisting of:
(a) the monoclonal antibody 5261;
(b) the monoclonal antibody 5378;
(c) the monoclonal antibody 5080;
(d) the monoclonal antibody 1476;
(e) the monoclonal antibody 3D2;
(f) a monoclonal antibody comprising a heavy chain variable region (VH) and a light chain variable region (VL) comprising the amino acid sequence set forth in SEQ ID NO: 13 and SEQ ID NO: 15, respectively;
(g) a monoclonal antibody comprising a VH and VL comprising the amino acid sequence set forth in SEQ ID NO: 13 and SEQ ID NO: 17, respectively;
(h) a monoclonal antibody comprising a VH and VL comprising the amino acid sequence set forth in SEQ ID NO: 3 and SEQ ID NO: 8, respectively;
(i) a monoclonal antibody comprising VH domain comprising a Chothia-Kabat heavy chain complementarity determining region-1 (VH-CDR1) amino acid sequence set forth in SEQ ID NO: 4; a Kabat heavy chain complementarity determining region-2 (VH-CDR2) amino acid sequence set forth in SEQ ID NO: 5; and a Kabat heavy chain complementarity determining region-3 (VH-CDR3) amino acid sequence set forth in SEQ ID NO: 6; and a VL domain comprising a Kabat light chain complementarity determining region-1 (VL-CDR1) amino acid sequence set forth in SEQ ID NO: 16 or 9; a Kabat light chain complementarity determining region-2 (VL-CDR2) amino acid sequence set forth in SEQ ID NO: 10; and a Kabat light chain complementarity determining region-3 (VL-CDR3) amino acid sequence set forth in SEQ ID NO: 11; and
(j) an antigen-binding fragment of any one of (a)-(i).

4. The antibody or antigen-binding fragment thereof of any one of claims 1-3, which is multispecific.

5. The antibody or antigen-binding fragment thereof of claim 4, which is bispecific.

6. The antibody or antigen-binding fragment thereof of any one of claims 1-5, which is an Fab fragment, an F(ab′)₂ fragment, an Fv fragment, or a scFv.

7. The antibody or antigen-binding fragment thereof of any one of claims 1-3, which is multivalent and comprises at least two heavy chains and at least two light chains.

8. The antibody or fragment thereof of any one of claims 1-6, which comprises a light chain constant region selected from the group consisting of a human kappa constant region and a human lambda constant region.

9. The antibody or fragment thereof of any one of claims 1-6, which comprises a human heavy chain constant region or fragment thereof selected from the group consisting of IgG1, IgG2, IgG3, IgG4, IgM, IgA1, IgA2, IgE, and IgD.

10. The antibody or fragment thereof of any one of claims 1-9, which specifically binds to a CXCL13 polypeptide or fragment thereof, or a CXCL13 variant polypeptide with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 5.7×10⁻¹² M, 8.4×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

11. The antibody or fragment thereof of claim 10, wherein said CXCL13 polypeptide or fragment thereof, or a CXCL13 variant polypeptide is human or murine.

12. The antibody or fragment thereof of claim 11, wherein said CXCL13 polypeptide or fragment thereof, or a CXCL13 variant polypeptide is human and said K_{D is} is about 5×10⁻⁸ to about 5×10⁻¹⁰.

13. The antibody or fragment thereof of claim 11, wherein said CXCL13 polypeptide or fragment thereof, or a CXCL13 variant polypeptide is murine and said K_{D} is about 5×10⁻⁸ to about 5×10⁻¹⁰.

14. The antibody or fragment thereof of any one of claims 1-13, which inhibits CXCL13 from binding to a CXCL13 receptor.

15. The antibody or fragment thereof of claim 14, wherein said CXCL13 receptor is CXCR5.

16. The antibody or fragment thereof of claim 14, wherein said CXCL13 receptor is CXCR3.

17. The antibody or fragment thereof of any one of claims 1-16, wherein said antibody or fragment thereof is humanized, primatized or chimeric.

18. The antibody or fragment thereof of any one of claims 1-17, further comprising a heterologous polypeptide fused thereto.

19. The antibody or fragment thereof of any one of claims 1-17, wherein said antibody or fragment thereof is conjugated to an agent selected from the group consisting of a cytotoxic agent, a therapeutic agent, a cytostatic agent, a biological toxin, a prodrug, a peptide, a protein, an enzyme, a virus, a lipid, a biological response modifier, a pharmaceutical agent, a lymphokine, a heterologous antibody or fragment thereof, a detectable label, polyethylene glycol (PEG), and a combination of two or more of any said agents.

20. A composition comprising the antibody or fragment thereof of any one of claims 1 to 19, and a pharmaceutically acceptable carrier.

21. The composition of claim 20, wherein said carrier is selected from the group consisting of saline, buffered saline, dextrose, water, glycerol, and combinations thereof.

22. A method for neutralizing CXCL13 in an animal, comprising administering to said animal an effective amount of the isolated antibody or fragment thereof of any one of claims 1-19 or the composition of claim 20 or 21.

23. A method for treating an autoimmune disease or an inflammatory disease in an animal in need of treatment, comprising administering to said animal an effective amount of the isolated antibody or fragment thereof of any one of claims 1-19 or the composition of claim 20 or 21.

24. The method of claim 23, wherein said autoimmune disease or said inflammatory disease is multiple sclerosis.

25. The method of claim 23, wherein said autoimmune disease or said inflammatory disease is Systemic Lupus Erythematosis (SLE).

26. The method of claim 23, wherein said autoimmune disease or said inflammatory disease is arthritis.

27. The method of claim 26, wherein said arthritis is rheumatoid arthritis.

28. A method for treating a cancer in an animal in need of treatment, comprising administering to said animal an effective amount of the isolated antibody or fragment thereof of any one of claims 1-19 or the composition of claim 20 or 21.

29. The method of claim 28, wherein said cancer is prostate or colon cancer.

30. A method for inhibiting gastric lymphoid follicles in an animal, comprising administering to said animal an effective amount of the isolated antibody or fragment thereof of any one of claims 1-19 or the composition of claim 20 or 21.

31. A method for preventing or treating mucosa-associated lymphoid tissue (MALT) lymphoma in an animal in need of prevention or treatment, comprising administering to said animal an effective amount of the isolated antibody or fragment thereof of any one of claims 1-19 or the composition of claim 20 or 21.

32. A method for preventing or treating a gastric or duodenal ulcer in an animal in need of prevention or treatment, comprising administering to said animal an effective amount of the isolated antibody or fragment thereof of any one of claims 1-19 or the composition of claim 20 or 21.

33. The method of any one of claims 30-32, wherein said animal has been infected with a Heliobacter bacterium.

34. The method of any one of claims 22-33, wherein said animal is a mammal.

35. The method of claim 34, wherein said mammal is a human.

36. An isolated polypeptide consisting of an epitope for binding an anti-CXCL13 antibody, wherein the epitope is selected from the group consisting of:
(a) an epitope comprising an amino acid sequence having at least 40% sequence identity to SEQ ID NO: 26;
(b) an epitope comprising an amino acid sequence identical to SEQ ID NO: 26, except for 9 or fewer amino acid substitutions; and
(c) an epitope sequence comprising the amino acid sequence SEQ ID NO: 26.

37. A method for producing an anti-CXCL13 antibody comprising immunizing an animal with a polypeptide according to claim 36.

38. The method of claim 37, further comprising isolating antibody-producing cells from the animal, fusing the antibody-producing cells with immortalized cells in culture to produce hybridoma cells, and isolating monoclonal antibodies from said hybridoma cells.


SEQUENCE LISTING (32 sequences)

SEQ ID NO: 1 - protein, 13 aa, Artificial Sequence
  Synthesized
HSLGKWLGHPDKF

SEQ ID NO: 2 - DNA, 369 bp, Artificial Sequence
  Synthesized
gaggtgcagcttcaggagtctggccctgggatattgcagccctcccagaccctcaatctgacttgttctttctctggattttcactgagcacttttggtatgggtgtaggctggattcgtcagccttcagggaagggtctggagtggctggcacacatttggtgggatgatgataggaggtataacccagccctgaagagtcggctcacaatctccaaggaaacctccaaaaaccaggtgttcctcaagatcgccaatgtggacactgcagatactgccacatactactgtactcgaatagcggggtattatggtagtagagactggtttgcttactggggccaagggaccacggtcaccgtctcctca

SEQ ID NO: 3 - protein, 123 aa, Artificial Sequence
  Synthesized
EVQLQESGPGILQPSQTLNLTCSFSGFSLSTFGMGVGWIRQPSGKGLEWLAHIWWDDDRRYNPALKSRLTISKETSKNQVFLKIANVDTADTATYYCTRIAGYYGSRDWFAYWGQGTTVTVSS

SEQ ID NO: 4 - protein, 7 aa, Artificial Sequence
  Synthesized
TFGMGVG

SEQ ID NO: 5 - protein, 16 aa, Artificial Sequence
  Synthesized
HIWWDDDRRYNPALKS

SEQ ID NO: 6 - protein, 13 aa, Artificial Sequence
  Synthesized
IAGYYGSRDWFAY

SEQ ID NO: 7 - DNA, 333 bp, Artificial Sequence
  Synthesized
gacattgtgctgacccagtctccagcttctttggctgtgtctctagggcagagggccaccatctcctgcagagccagcgaaagtgttgataattctggcattagttttatgcactggtaccagcagaaaccaggacagccacccaaactcctcatctttcgtgcatccgacctagaatctgggatccctgccaggttcagtggcagtgggtctaggacagacttcaccctcaccgttaatcctgtggagactgatgatgttgcaacctatttctgtcagcaaagtaataaggatccgtggacgttcggtggaggcaccaagctcgagatcaaa

SEQ ID NO: 8 - protein, 111 aa, Artificial Sequence
  Synthesized
DIVLTQSPASLAVSLGQRATISCRASESVDNSGISFMHWYQQKPGQPPKLLIFRASDLESGIPARFSGSGSRTDFTLTVNPVETDDVATYFCQQSNKDPWTFGGGTKLEIK

SEQ ID NO: 9 - protein, 15 aa, Artificial Sequence
  Synthesized
RASESVDNSGISFMH

SEQ ID NO: 10 - protein, 7 aa, Artificial Sequence
  Synthesized
RASDLES

SEQ ID NO: 11 - protein, 9 aa, Artificial Sequence
  Synthesized
QQSNKDPWT

SEQ ID NO: 12 - DNA, 369 bp, Artificial sequence
  Synthesized
caggtgcagctgcaggagagcggcccaggcctggtgaagcctagcgagaccctgagcctcacctgcaccgtcagcggctttagcctgagcacctttggcatgggcgtgggctggattagacagcctccaggcaagggcctggagtggattgcacacatttggtgggatgatgataggagatataacccagccctgaagagcagagtgaccatcagcaaggacaccagcaagaaccagttcagcctgaagctgagcagcgtgaccgctgccgacaccgccgtgtattactgtgccagaatcgccggctattatggcagcagagactggtttgcctactggggccaaggcaccacggtcaccgtctcctca

SEQ ID NO: 13 - protein, 123 aa, Artificial Sequence
  Synthesized
QVQLQESGPGLVKPSETLSLTCTVSGFSLSTFGMGVGWIRQPPGKGLEWIAHIWWDDDRRYNPALKSRVTISKDTSKNQFSLKLSSVTAADTAVYYCARIAGYYGSRDWFAYWGQGTTVTVSS

SEQ ID NO: 14 - DNA, 333 bp, Artificial Sequence
  Synthesized
gacatccagatgacccagtctccatcctccctgtctgcatctgtgggagacagagtcaccatcacttgcagagccagcgaaagtgttgataatatgggcattagttttatgcactggtatcagcagaaaccagggaaagcccctaagctcctgatctttagagcatccgacctggaatctggggtcccatcaaggttcagtggcagtggatctgggacagatttcactctcaccatcagcagtctgcaacctgaagattttgcaacttactactgtcagcaaagtaataaggatccctggaccttcggccaagggaccaagctcgagatcaaa

SEQ ID NO: 15 - protein, 111 aa, Artificial Sequence
  Synthesized
DIQMTQSPSSLSASVGDRVTITCRASESVDNMGISFMHWYQQKPGKAPKLLIFRASDLESGVPSRFSGSGSGTDFTLTISSLQPEDFATYYCQQSNKDPWTFGQGTKLEIK

SEQ ID NO: 16 - protein, 15 aa, Artificial Sequence
  Synthesized
RASESVDNMGISFMH

SEQ ID NO: 17 - protein, 111 aa, Artificial Sequence
  Synthesized
DIQMTQSPSSLSASVGDRVTITCRASESVDNSGISFMHWYQQKPGKAPKLLIFRASDLESGVPSGFSGSGSRTDFTLTISSLQPEDFATYYCQQSNKDPWTFGQGTKLEIK

SEQ ID NO: 18 - DNA, 333 bp, Artificial Sequence
  Synthesized
gacatccagatgacccagtctccatcctccctgtctgcatctgtgggagacagagtcaccatcacttgcagagccagcgaaagtgttgataattctggcattagttttatgcactggtatcagcagaaaccagggaaagcccctaagctcctgatctttagagcatccgacctggaatctggggtcccatcagggttcagtggcagtggatctaggacagatttcactctcaccatcagcagtctgcaacctgaagattttgcaacttactactgtcagcaaagtaataaggatccctggaccttcggccaagggaccaagctcgagatcaaa

SEQ ID NO: 19 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 20 - DNA, 1219 bp, Homo sapiens
gagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccaggaaaaagaacttccccatacaaataagcatgagactatgtaaaaataaccttgcagaagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtggggggcggggccggggggactctggtatctaattctttaatgattcctataaatctaatgacattcaataaagttgagcaaacattttacttaaaaaaaaaaaaaaaaaa

SEQ ID NO: 21 - protein, 109 aa, Mus musculus
MRLSTATLLLLLASCLSPGHGILEAHYTNLKCRCSGVISTVVGLNIIDRIQVTPPGNGCPKTEVVIWTKMKKVICVNPRAKWLQRLLRHVQSKSLSSTPQAPVSKRRAA

SEQ ID NO: 22 - DNA, 1162 bp, Mus musculus
gagctaaaggttgaactccacctccaggcagaatgaggctcagcacagcaacgctgcttctcctcctggccagctgcctctctccaggccacggtattctggaagcccattacacaaacttaaaatgtaggtgttctggagtgatttcaactgttgtcggtctaaacatcatagatcggattcaagttacgccccctgggaatggctgccccaaaactgaagttgtgatctggaccaagatgaagaaagttatatgtgtgaatcctcgtgccaaatggttacaaagattattaagacatgtccaaagcaaaagtctgtcttcaactccccaagctccagtgagtaagagaagagctgcctgaagccactatcatctcaaaagacacacctgcacctttttttttatccctgctctgaattttagatatgttcttagttaaagaatttccaagaaaataactcccctctacaaacaaacatgactgtaggtaaaacaaagcaaaaacaaacaagcaaacaaacaaactaaaaaaaacccaatcctgcaggagctgagagggaatgctcaagctccgttgcatacccaacccacatccttgttccttaagaaaggctatttgagaacaggcatttagtgacaacccacttcagatgcatgtggtaatagatctgttgtttaatgttaaactatcctagattgtcgaggaatgaaaaacctacatgtcaaatgtgaacttgtagctcgtactaacaagaggtttgcgagatggacttcagttattttgcacccttgtaaaacgcaggcttccaaaatagtctccagaaggttcctgggaagctggtgcaatgccatcatgaggtggtgcaaagcaggtctcctttagagaaaagcttcctgggggaaacagtcctactttgaaaggttgcttgtataagatttattgtcttgcattaaaaccagtaacaattgaaagatcctcagcttaaaggtccaggctcttcagcagtatacaaatatattcctttgcactgtgaccctgatgatctatttttattattcatatctttcacacagacaaaataccagcctcttgtatcagattctttaatgtttcctattcatctggtgtcattcaataaatgtaatcaaatgttttgctta

SEQ ID NO: 23 - protein, 82 aa, Macaca fascicularis
VLEVYYTHLRCRCVQESSVFIPRRFIDRIQISPRGNGCPRKEIIVWKKNKSVVCVDPQAEWIQRIMEMLRKKSSSTPPVPVF

SEQ ID NO: 24 - DNA, 21 bp, Artificial Sequence
  Synthesized
ttgggaggctttgtctttcca

SEQ ID NO: 25 - DNA, 21 bp, Artificial Sequence
  Synthesized
gattagctctgcctcgcggct

SEQ ID NO: 26 - protein, 15 aa, Homo sapiens
YTSLRCRCVQESSVF

SEQ ID NO: 27 - DNA, 2902 bp, Homo sapiens
  CDS (177)...(1295)
aaaaaaaaaaagtgatgagttgtgaggcaggtcgcggccctactgcctcaggagacgatgcgcagctcatttgcttaaatttgcagctgacggctgccacctctctagaggcacctggcggggagcctctcaacataagacagtgaccagtctggtgactcacagccggcacagccatgaactacccgctaacgctggaaatggacctcgagaacctggaggacctgttctgggaactggacagattggacaactataacgacacctccctggtggaaaatcatctctgccctgccacagaggggcccctcatggcctccttcaaggccgtgttcgtgcccgtggcctacagcctcatcttcctcctgggcgtgatcggcaacgtcctggtgctggtgatcctggagcggcaccggcagacacgcagttccacggagaccttcctgttccacctggccgtggccgacctcctgctggtcttcatcttgccctttgccgtggccgagggctctgtgggctgggtcctggggaccttcctctgcaaaactgtgattgccctgcacaaagtcaacttctactgcagcagcctgctcctggcctgcatcgccgtggaccgctacctggccattgtccacgccgtccatgcctaccgccaccgccgcctcctctccatccacatcacctgtgggaccatctggctggtgggcttcctccttgccttgccagagattctcttcgccaaagtcagccaaggccatcacaacaactccctgccacgttgcaccttctcccaagagaaccaagcagaaacgcatgcctggttcacctcccgattcctctaccatgtggcgggattcctgctgcccatgctggtgatgggctggtgctacgtgggggtagtgcacaggttgcgccaggcccagcggcgccctcagcggcagaaggcagtcagggtggccatcctggtgacaagcatcttcttcctctgctggtcaccctaccacatcgtcatcttcctggacaccctggcgaggctgaaggccgtggacaatacctgcaagctgaatggctctctccccgtggccatcaccatgtgtgagttcctgggcctggcccactgctgcctcaaccccatgctctacactttcgccggcgtgaagttccgcagtgacctgtcgcggctcctgacgaagctgggctgtaccggccctgcctccctgtgccagctcttccctagctggcgcaggagcagtctctctgagtcagagaatgccacctctctcaccacgttctaggtcccagtgtccccttttattgctgcttttccttggggcaggcagtgatgctggatgctccttccaacaggagctgggatcctaagggctcaccgtggctaagagtgtcctaggagtatcctcatttggggtagctagaggaaccaacccccatttctagaacatccctgccagctcttctgccggccctggggctaggctggagcccagggagcggaaagcagctcaaaggcacagtgaaggctgtccttacccatctgcacccccctgggctgagagaacctcacgcacctcccatcctaatcatccaatgctcaagaaacaacttctacttctgcccttgccaacggagagcgcctgcccctcccagaacacactccatcagcttaggggctgctgacctccacagcttcccctctctcctcctgcccacctgtcaaacaaagccagaagctgagcaccaggggatgagtggaggttaaggctgaggaaaggccagctggcagcagagtgtggccttcggacaactcagtccctaaaaacacagacattctgccaggcccccaagcctgcagtcatcttgaccaagcaggaagctcagactggttgagttcaggtagctgcccctggctctgaccgaaacagcgctgggtccaccccatgtcaccggatcctgggtggtctgcaggcagggctgactctaggtgcccttggaggccagccagtgacctgaggaagcgtgaaggccgagaagcaagaaagaaacccgacagagggaagaaaagagctttcttcccgaaccccaaggagggagatggatcaatcaaacccggcggtcccctccgccaggcgagatggggtggggtggagaactcctagggtggctgggtccaggggatgggaggttgtgggcattgatggggaaggaggctggcttgtcccctcctcactcccttcccataagctatagacccgaggaaactcagagtcggaacggagaaaggtggactggaaggggcccgtgggagtcatctcaaccatcccctccgtggcatcaccttaggcagggaagtgtaagaaacacactgaggcagggaagtccccaggccccaggaagccgtgccctgcccccgtgaggatgtcactcagatggaaccgcaggaagctgctccgtgcttgtttgctcacctggggtgtgggaggcccgtccggcagttctgggtgctccctaccacctccccagcctttgatcaggtggggagtcagggacccctgcccttgtcccactcaagccaagcagccaagctccttgggaggccccactggggaaataacagctgtggctcacgtgagagtgtcttcacggcaggacaacgaggaagccctaagacgtcccttttttctctgagtatctcctcgcaagctgggtaatcgatgggggagtctgaagcagatgcaaagaggcaagaggctggattttgaattttctttttaataaaaaggcacctataaaacaggtcaatacagtacaggcagcacagagacccccggaacaagcctaaaaattgtttcaaaataaaaaccaagaagatgtcttcacatattgt

SEQ ID NO: 28 - protein, 372 aa, Homo sapiens
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 29 - DNA, 2614 bp, Mus musculus
  CDS (44)...(1168)
cgacatcagacagtgaccagcctggtgacgcagagctgcagctatgaactacccactaaccctggacatgggctccatcacatacaatatggatgacctgtacaaggaactggccttctacagtaacagcacggagattcccctacaggacagtaacttctgctctacagtcgagggacccttactgacgtcctttaaggcggtattcatgcctgtggcctacagcctcatcttcctcctgggtatgatgggaaacatcctggtgctggtaatcctggagaggcaccggcacactcggagctcaaccgagaccttcctgttccacctcgcagtagccgaccttctcttagtcttcatcctgccttttgcagtggctgagggctctgtgggttgggtcctagggaccttcctctgcaaaactgtgatcgctctgcacaagatcaatttctactgcagcagcctgctgctggcctgtatagctgtagaccggtacctagccatcgtccatgctgttcacgcctaccgccgccgtcgactcctctccatccacatcacctgcacggccatttggctggccggcttcctgttcgccttaccggaactcctctttgccaaggttggccaacctcataacaacgactccttaccacagtgcaccttctcccaggaaaacgaagcggaaactagagcctggttcacctcccgtttcctctaccacatcgggggcttcctactaccgatgcttgtgatgggatggtgttacgtgggcgtggtccacaggctactgcaggcccagcggcgccctcagcggcagaaggcggtcagggtggccattttagtgacaagcattttcttcctctgctggtcgccctaccacattgtcatcttcctagatacactggagaggctgaaggctgtgaatagcagctgcgagctgagtggctatctctctgtggccatcaccttgtgtgaattcctgggcctggcacactgctgtctcaatcccatgctctacaccttcgctggcgtaaagttccgcagtgacctctctcggcttctgaccaagctgggctgtgctggcccggcctccctttgccaacttttccccaactggcgcaagagtagtctctctgagtcagagaatgctacttccctcaccaccttctagatcccggaagtctcggggcccctgtctgtttctgttttccttgggaggataaagtggtggcggaacccatccaactcgagcttgggccagtgtccccagatgggaaagctagataaactctctcaaactttcccaaaggggaaagcagcccaaaggcaaagcaagctatatccaggccacctgtatcaccttagatgaagagaactccatacacctcccatcctaaccagctaaagctaagctcagctttatttcttcctggccatagggacaaccacctctgctgtggcccacagtctcatcttcctcctgattatgagcccagactctcctcccagaatgtattccatcatcttaaagactactggctgccacagctacccaccactcctataccacagaggaatagccagctggcggcggcagactatggccttaatgtgcctgtctcataaatacagacttcatgccagaccttcaaccgtgcctttctcttaaccaagcagaaagctgaaaccgatctactttaggtagctgtctggttccaacctaaccagcattgggtcagccccatgttactggatcttggattacagactgagggcaagttccagaaggttctggaagctagccagtatcctaagaagagtaaagggcaagccagcaggaaagaggcccagtggaaaagtggaaagacaccttttccaggctctaaggaagaacaagtaaaaatcaaacccagctgtcttctccacccaatgtaccaaagcttacagactggtggggaaatgagatccagggccctcgtggattctacgcaccaatggggaaggaagccaacttgcctggggaaagcaagatagcaaagtggtcctagcctcgagagaggggacacctagctaagagaatgacgacagaggttcctgtcttcattaggcagaggcaatataagaagccaacctgggcaggcaagtcctcaaaccccaggaaggcagtaccctgcccctgggagggtaccactcacatggaaccagaggaagctgctccatgcatacataggggaagttagcaggcaattctgagctcggcttcctcccagccaccgatctgggggcgtgggggtaggaagcagagttgcctagtaccactcaagccaaccgtacaagctccctgggggatcccactggggaaaccaatgctatagcttcagagactgtatcctcattgcagaaccgtgaagacacctggggacccccttttctgctcccagcatccaacaaccagctgggaagaggcaaaccgggcacagaaataaaaatgcaagagatggcatttttgaattttctctttttaataaaaaggcacctataaaacaggtcaatacaggcagagacccccggaacaagcctaaaaagtgtttcaaaataaaaacaggaagatgtcttc

SEQ ID NO: 30 - protein, 374 aa, Mus musculus
MNYPLTLDMGSITYNMDDLYKELAFYSNSTEIPLQDSNFCSTVEGPLLTSFKAVFMPVAYSLIFLLGMMGNILVLVILERHRHTRSSTETFLFHLAVADLLLVFILPFAVAEGSVGWVLGTFLCKTVIALHKINFYCSSLLLACIAVDRYLAIVHAVHAYRRRRLLSIHITCTAIWLAGFLFALPELLFAKVGQPHNNDSLPQCTFSQENEAETRAWFTSRFLYHIGGFLLPMLVMGWCYVGVVHRLLQAQRRPQRQKAVRVAILVTSIFFLCWSPYHIVIFLDTLERLKAVNSSCELSGYLSVAITLCEFLGLAHCCLNPMLYTFAGVKFRSDLSRLLTKLGCAGPASLCQLFPNWRKSSLSESENATSLTTF

SEQ ID NO: 31 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 32 - protein, 415 aa, Homo sapiens
MELRKYGPGRLAGTVIGGAAQSKSQTKSDSITKEFLPGLYTAPSSPFPPSQVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL